(19) United States
(12) Patent Application Publication
(10) Pub. No.: US 2009/0028836 A1
(43) Pub. Date: Jan. 29, 2009

(54) STEM AND PROGENITOR CELL EXPANSION BY EVI, EVI-LIKE GENES AND SETBP1

(76) Inventors: Christof von Kalle, Heidelberg (DE); Manfred Schmidt, Heidelberg (DE); Manuel Grez, Frankfurt (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/948,920

(22) Filed: Nov. 30, 2007

Related U.S. Application Data
(63) Continuation of application No. PCT/US2006/021413, filed on Jun. 1, 2006.
(60) Provisional application No. 60/686,963, filed on Jun. 1, 2005.

(51) Int. Cl. A61K 35/12; C12N 15/87; C12Q 1/68; C12N 15/11; C12N 5/06; A61P 43/00

(52) U.S. Cl. 424/93.21; 435/455; 435/6; 536/23.1; 435/375

(57) ABSTRACT

A method of increasing cell proliferation by modulating levels of EVI and related genes. Activation of EVI-1, PRDM16, or SETBP1 can increase the proliferation rate, self renewal and/or in vitro and/or in vivo survival and/or engraftment of human cells, either in vitro or in vivo. The gene modulation can be performed by various means, including traditional cloning methods and retroviral-based gene activation methods. The method can also be used to more efficiently deliver gene-corrected cells to a patient in need of treatment.

### RELATED APPLICATIONS

This application is a continuation under 35 U.S.C. § 365 (c) claiming the benefit of the filing date of PCT Application No. PCT/US2006/021413 designating the United States, filed Jun. 1, 2006. The PCT Application was published in English as WO 2007/008309 on Jan. 18, 2007, and claims the benefit of the earlier filing date of U.S. Provisional Application Ser. No. 60/686,963, filed Jun. 1, 2005. The contents of the U.S. Provisional Application Ser. No. 60/686,963 and the international application No. PCT/US2006/021413 including the publication WO 2007/008309 are incorporated herein by reference in their entirety.

### REFERENCE TO SEQUENCE LISTING

The present application is being filed along with a Sequence Listing in electronic format. The Sequence Listing is provided as a file entitled SEQLIST_LOMAU_{—}170.TXT, created Nov. 29, 2007, which is 4 Kb in size. The information in the electronic format of the Sequence Listing is incorporated herein by reference in its entirety.

### FIELD OF THE INVENTION

The invention relates to the field of cell biology and gene therapy. In particular, the invention relates to methods of increasing cell proliferation in vivo or in culture by modulating expression of certain regulatory genes.

### BACKGROUND OF THE INVENTION

Gene therapy methods are currently being pursued for the treatment of a variety of human diseases. Retroviral vectors, for example, have been successfully used in clinical gene therapy trials to treat severe combined immunodeficiencies (SCID), where gene correction conferred a selective advantage to lymphocytes (Cavazzana-Calvo, et al. (2000) Science 288:669-672; Aiuti, et al. (2002) Science 296:2410-2413; Gaspar, et al. (2004) Lancet 364:2181-2187, each of the foregoing which is hereby incorporated by reference in its entirety). However, in inherited leukocyte disorders without a selective advantage by gene correction, human gene therapy has been less effective (Kohn, et al. (1998) Nature Med. 4:775-780; Malech, et al. (1997) Proc. Natl. Acad. Sci. U.S.A. 94:12133-12138, each of the foregoing which is hereby incorporated by reference in its entirety).

While insertion induced oncogenesis has been reported for wild type retroviruses (Hayward, et al. (1981) Nature 290: 475-480; Selten, et al. (1984) Embo J. 3:3215-22, each of the foregoing which is hereby incorporated by reference in its entirety) and related replication competent vectors (Dudley, J. P. (2003) Trends Mol Med 9:43-45, which is hereby incorporated by reference in its entirety), retrovirus vector based gene therapy with non-replicating vectors was thought to lead to random monoallelic integration without relevant biological consequences (Coffin, et al. (1997) Retroviruses. Plainview, N.Y.: Cold Spring Harbor Laboratory Press; Moolten, et al. (1992) Hum Gene Ther 3:479-486, each of the foregoing which is hereby incorporated by reference in its entirety).

Although gene therapy methods, in theory, should provide useful methods for the treatment of many types of human diseases, several problems currently exist. One problem with current gene therapy methods is that gene-corrected cells growing in culture or in vivo, often do not expand rapidly. If these cultures could be treated so as to expand more rapidly, the gene therapy process could become more efficient and more likely to succeed. Thus, methods that are capable of increasing the rate of expansion of cells, such as mammalian hematopoietic cells, either in vitro or in vivo, would be useful to improve the effectiveness of a variety of gene therapy methods. Likewise, increasing the rate of expansion, and/or favoring the persistence of mammalian hematopoietic stem cells or progenitor cells, in vitro or in vivo, would be of great value independently of gene therapy methods and indications, including, but not restricted to, stem cell transplantation with and without ex vivo modification.

### SUMMARY OF THE INVENTION

In some embodiments of the present invention, a method of increasing cell proliferation by modulating levels of EVI and related genes is provided. Activation of EVI-1, PRDM16, or SETBP1 can increase the proliferation rate, self renewal and/or in vitro and/or in vivo survival and/or engraftment of human cells, either in vitro or in vivo. The gene modulation can be performed by various means, including traditional cloning methods and retroviral-based gene activation methods. The method can also be used to more efficiently deliver gene-corrected cells to a patient in need of treatment.

In some embodiments of the present invention, a method of expanding cells is provided, by obtaining at least one cell from a patient, transfecting, infecting or transducing said cell with a retroviral or nonintegrating vector, such that cell entry and/or integration of the vector promotes proliferation, persistence, or selective advantage of the cell, allowing the transfected cell to proliferate, reinfusing a plurality of proliferated transfected cells into said patient, and allowing said proliferated cells to expand further in the patient. The transfected cell can have characteristics of a cell such as, for example, a hematopoietic progenitor cell, a hematopoietic stem cell, or a stem cell. The method can be used to treat a patient with a hematopoietic or other treatable disease. The vector can also have a sequence for correction or modification of a defective or deleterious gene.

In additional embodiments of the present invention, a method of increasing cell proliferation in a mammalian cell is provided, by obtaining a cell, contacting the cell with a nucleic acid sequence encoding a protein selected from the group consisting of EVI-1, PRDM16, SETBP1, and a fragment thereof, allowing said nucleic acid to enter the cell, and allowing said cell to proliferate, where the cell having the nucleic acid proliferates at an increased rate compared to a cell that has not been contacted with the nucleic acid sequence. The proliferation can occur, for example, in a cell culture, ex vivo, or in vivo. The nucleic acid can integrate, for example, into the chromosomal DNA. The nucleic acid can be present, for example, in the cytoplasm of the cell. The nucleic acid can be operably linked to a promoter. The nucleic acid can be constitutively expressed. The expression of the nucleic acid can be inducible, for example, by an exogenously added agent. The nucleic acid can be present in a vector, such as, for example, a viral vector. The nucleic acid can be expressed for a number of division cycles such as, for example, about 1, 3, 5, 8, 10, 13, 17, or 20 division cycles, then expression can decrease or stop thereafter. The cell can have characteristics of a cell selected from the group consisting of a hematopoietic stem cell, hematopoietic progenitor cell, a stem cell, an embryonic stem cell, an adult stem cell, a multipotent stem cell, and a myelopoietic stem cell.

In a further embodiment of the present invention, a method of expansion of a gene-corrected cell is provided, by obtaining a cell in need of gene correction, transfecting the cell with a functional copy of a the gene in need of correction, transfecting the cell with a copy of a nucleic acid encoding a polypeptide sequence selected from the group consisting of EVI-1, PRDM16, SETBP1, and a fragment thereof; and allowing the cell to proliferate in culture.

In a further embodiment of the present invention, a method of forming a bodily tissue having gene corrected cells is provided, by obtaining a cell in need of gene correction, transfecting the cell with a functional copy of a the gene in need of correction, transfecting the cell with a copy of a nucleic acid encoding a polypeptide sequence selected from the group consisting of EVI-1, PRDM16, SETBP1, and a fragment thereof, allowing the cell to proliferate in culture, and treating the cell culture to allow formation of a bodily tissue.

In a further embodiment of the present invention, a method of identifying a gene is provided, the modulation of which increases the proliferation rate of a cell, by obtaining a sample of cells from a patient having previously received a therapeutic transfection with a nucleic acid sequence, identifying positions of nucleic acid insertion in the cells from the sample, identifying a favorable insertion site based upon disproportional representation of the site in the population of transfected cells, and identifying a gene associated with the insertion site.

In a yet further embodiment of the present invention, a nucleic acid integration region is provided, that, when insertionally modulated, results in increased hematopoietic cell proliferation, as is selected from the EVI-1 gene, the PRDM16 gene, and the SETBP1 gene.

In a further embodiment of the present invention, a nucleic acid sequence whose modulation of expression is associated with the increased proliferation of hematopoietic cells is provided, selected from the following group: MGC10731, PADI4, CDA, CDW52, ZBTB8, AK2, FLJ32112, TACSTD2, FLJ13150, MGC24133, NOTCH2, NOHMA, EST1B, PBX1, PLA2G4A, HRPT2, ATP6V1G3, PTPRC, NUCKS, CABC1, LOC339789, PRKCE, AFTIPHILIN, NAGK, MARCH7, DHRS9, PRKRA, SESTD1, MGC42174, CMKOR1, TBC1D5, THRB, MAP4, IFRD2, ARHGEF3, FOXP1, ZBTB20, EAF2, MGLL, PLXND1, SLC9A9, SELT, CCNL1, MDS1, BCL6, MIST, STIM2, TEC, OCIAD1, FLJ10808, SEPT11, PRKG2, MLLT2, PGDS, MANBA, SRY1, SET7, MAML3, DCTD, CARF, IRF2, AHRR, POLS, ROPN1L, FLJ10246, IPO11, C2GNT3, SSBP2, EDIL3, SIAT8D, FLJ20125, GNB2L1, C6orf105, JARID2, C6 orf32, HCG9, MGC57858, TBCC, SENP6, BACH2, REPS1, HDAC9, OSBPL3, HOXA7, CALN1, FKBP6, NCF1, HIP1, GNAI7, ZKSCAN1, MGC50844, LOC346673, CHRM2, ZH3HAV1, REPIN1, SMARCD3, CTSB, ADAM28, LYN, YTHDF3, SMARCA2, C9orf93, NPR2, BTEB1, ALDH1A1, AUH, C9orf3, WDR31, CEP1, GSN, RABGAP1, ZNF79, CUGBP2, C10orf7, PTPLA, PLXD2, ACBD5, PRKG1, MYST4, IFIT1, C10orf129, CUEDC2, FAM45A, GRK5, OR52NI, OR2AG2, ZNF143, C11orf8, LMO2, NGL-1, DGKZ, NR1H3, KBTBD4, C1QTNF4, MGC5395, ARRB1, FLJ23441, FGIF, MAML2, LOC196264, HSPC063, ELKS, CACNA2D4, CHD4, EPS8, LRMP, NEUROD4, RNF41, FAM19A2, RASSF3, PAMC1, PLXNC1, DAP13, MGC4170, FLJ40142, JIK, CDK2AP1, GPR133, PCDH9, C13orf25, ABHD4, AP4S1, MIA2, RPS29, PSMC6, RTN1, MED6, C14orf43, C14orf118, RPS6KA5, GNG2, PAK6, B2M, ATP8B4, TRIP4, CSK, MESDC1, RKHD3, AKAP13, DET1, DKFZp547K1113, SV2B, LRRK1, CHSY1, TRAF7, ZNF205, ABCC1, THUMPD1, IL21R, MGC2474, N4BP1, SLIC1, CDH9, GPR56, ATBF1, ZNRF1, CMIP, MGC22001, C17orf31, SAT2, ADORA2B, TRPV2, NF1, LOC117584, MLLT6, STAT5A, STAT3, HOXB3, HLF, MAP3K3, SCN4A, ABCA10, EPB41L3, ZNF521, RNF125, SETBP1, FLJ20071, CDH7, MBP, MBP, NFATC1, GAMT, MOBKL2A, NFIC, CALR, GPSN2, ZNF382, EGLN2, PNKP, LAIR1, ZNF579, SOX12, C20orf30, PLCB1, SNX5, LOC200261, ZNF336, BAK1, SPAG4L, EPB411L1, NCOA3, KIAA1404, STIMN3, CBR3, DYRK1A, CSTB, C22orf14, UPB1, MN1, XBP1, C22orf19, RBM9, MYH9, TXN2, PSCD4, UNC84B, FLJ2544, ZCCHC5, MST4, IDS, UTY, SKI, PRDM16, PARK7, CHC1, ZMYM1, INPP5B, GLIS1, SLC27A3, ASH1L, SLAMF1, PBX1, CGI-49, ELYS, RNF144, FAM49A, FLJ21069, SFRS7, SPTBN1, TMEM17, ARHGAP25, FLJ20558, CAPG, PTPN18, RBMS1, LOC91526, KLF7, FLJ23861, CMKOR1, CRBN, ITPR1, RAFTLIN, TNA, CCDC12, FHIT, VGL-3, PPM1L, EVI-1, MDS1, HDSH3TC1, DHX15, TMEM33, CXCL3, EPGN, LRBA, FLJ25371, CPE, POLS, PTGER4, LHFPL2, C5orf12, CETN3, PHF15, PFDN1, KIAA0555, GNB2L1, HLA-E, SLC17A5, UBE2J1, BACH2, HIVEP2, SNX8, TRIAD3, RAC1, ARL4A, ELMO1, BLVRA, SUNC1, ABCA13, GTF2IRD1, RSBN1L, ADAM22, MLL5, IMMP2L, SEC8L1, FLJ12571, CUL1, ANGPT1, DEPDC6, EPPK1, MLANA, MLLT3, SMU1, TLE4, C9 orf3, ABCA1, STOM, RABGAP1, NEK6, NR5A1, MGC20262, FLJ20433, MAP3K8, ARHGAP22, C10orf72, TACR2, NKX2, OBFC1, VTI1A, ABLIM1, FLJ14213, MS4A3, B3GNT6, NADSYN1, CENTD2, MAML2, ATP5L, FLI1, CACNA1C, HEBP1, MLSTD1, IPO8, ARID2, SLC38A1, KRT7, USP15, KIAA1040, WIF1, CGI-119, DUSP6, FLJ11259, CMKLR1, SSH1, TPCN1, FLJ42957, JIK, FLT3, TPT1, FNDC3, ARHGAP5, ARF6, GPHN, C14orf4, STN2, PPP2R5C, CDC42BPB, CEP152, OAZ2, AKAP13, CHSY1, CRAMP1L, MHC2TA, NPIP, SPN, MMP2, DKFZp434I099, SIAT4B, PLCG2, MYO1C, C17orf31, MGC51025, WSB1, TRAF4, SSH2, HCA66, RFFL, DUSP14, TCF2, ZNF652, STXBP4, HLF, MSI2, VMP1, HELZ, TREM5, RAB37, SEC14L1, SEPT9, BIRC5, PSCD1, MGC4368, NDUFV2, C18orf25, ATP8B1, CDH7, FLJ44881, NFATC1, C19 orf35, GNG7, MATK, C3, ZNF358, LYL1, F2RL3, ZNF253, ZNF429, KIAA1533, U2AF1L3, GMFG, BC-2, C20orf30, PLCB1, LOC200261, C20orf112, ADA, PREX1, C21orf34, C21orf42, ERG, ABCG1, MN1, HORMAD2, LOC113826, C22orf1, EFHC2, SYLT4, MGC27005, FHL1, GAB3, and CSF2RA.

In a further embodiment of the present invention, a method of identifying a favorable insertion site of a nucleic acid sequence in a proliferating cell culture is provided, by transfecting a cell sample with a nucleic acid sequence, allowing cell proliferation to occur, determining at least one main insertion site of the nucleic acid using linear amplification mediated PCR (LAM-PCR) over time, using the at least one main insertion site to predict the location of at least one main insertion site of another cell sample transfected with a substantially similar nucleic acid sequence over a similar time period, obtaining a sample of cells from a patient having previously received a therapeutic transfection with a nucleic acid sequence, identifying positions of nucleic acid insertion in the cells from the sample, and identifying a favorable insertion site based upon disproportional representation of the site in the population of transfected cells.

In a further embodiment of the present invention, a method of expansion of a cell is provided, comprising contacting the cell with a polypeptide selected from the group consisting of: an EVI-1 polypeptide, a PRDM16 polypeptide, a SETBP1 polypeptide, a fragment thereof, or a synthetic peptide derivative thereof.

In a further embodiment of the present invention, a method of treating an individual having a disease caused by a mutated gene or an inappropriately expressed gene is provided, by administered cells that have been corrected for the gene of interest, where the cells also have an increased level of at least one of an EVI-1 polypeptide, a PRDM16 polypeptide, or a SETBP1 polypeptide. In additional embodiments of the present invention, the disease is chronic granulomatous disease (CGD).

In additional embodiments of the present invention, a method of improving gene therapy is provided, by treating an individual with gene-corrected cells that have also been altered to have increased levels of at least one of the following polypeptides: an EVI-1 polypeptide, a PRDM16 polypeptide, or a SETBP1 polypeptide.

### BRIEF DESCRIPTION OF THE DRAWINGS

FIG. 1 shows hematopoietic reconstitution and gene marking in patient P1 after gene therapy. Cell counts are shown both before and after gene therapy. Absolute neutrophil counts are measured against the right y-axis, while counts of helper T cells (CD4+CD3+), cytotoxic T cells (CD8+CD3+) and B cells (CD19+) are measured against the left y-axis.

FIG. 2 shows hematopoietic reconstitution and gene marking in patient P2 after gene therapy. Cell counts are shown both before and after gene therapy. Absolute neutrophil counts are measured against the right y-axis, while counts of helper T cells (CD4+CD3+), cytotoxic T cells (CD8+CD3+) and B cells (CD19+) are measured against the left y-axis.

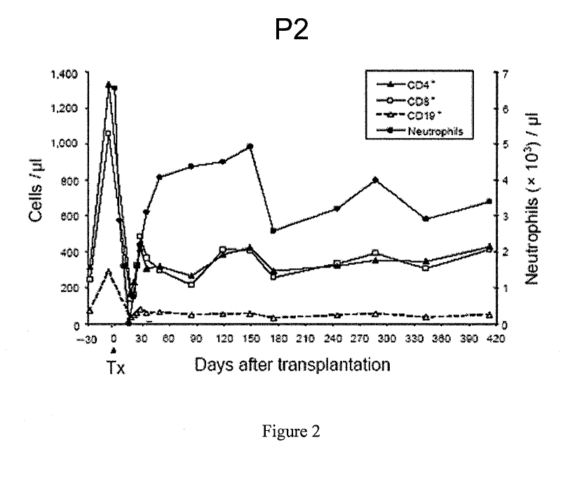

FIG. 3 illustrates quantification of gene-modified cells in peripheral blood leukocytes (PBL), granulocytes (CD15+), T-cells (CD3+) and B cells (CD19+) for patient P1 by quantitative PCR (QPCR).

FIG. 4 illustrates quantification of gene-modified cells in peripheral blood leukocytes (PBL), granulocytes (CD15+), T-cells (CD3+) and B cells (CD19+) for patient P2 by quantitative PCR (QPCR).

FIG. 5 shows gene marking in CFCs derived from bone marrow aspirates of patient P1 (days +122 and +381). Vector-containing CFCs were detected by PCR using primers specific for cDNA encoding gp91^{phox}. Input DNA was controlled by amplification of sequences derived from the human erythropoietin receptor (hEPO-R).

FIG. 6 shows gene marking in CFCs derived from bone marrow aspirates of patient P2 (days +119 and +245). Vector-containing CFCs were detected by PCR using primers specific for cDNA encoding gp91^{phox}. Input DNA was controlled by amplification of sequences derived from the human erythropoietin receptor (hEPO-R).

FIG. 7 illustrates the RIS distribution of retroviral vector insertions from 30 kb upstream to 5 kb downstream of RefSeq genes in patient P1. Absolute numbers of integrations into the 3 common integration site (CIS) related RefSeq genes MDS1/EVI-1, PRDM16 and SETBP1 are shown as black bars, while integrations into non CIS-related genes are shown in grey. The insertions are listed according to their location within the affected gene expressed as the percentage of the overall length of the gene. The last column summarizes all integrations up to 5 kb downstream of a gene. Up, upstream of the start of transcription; down, downstream of the RefSeq gene. TSS, transcription start site.

FIG. 8 illustrates the RIS distribution of retroviral vector insertions from 30 kb up- to 5 kb downstream of RefSeq genes in patient P2. Absolute numbers of integrations into the 3 CIS related RefSeq genes MDS1/EVI-1, PRDM16 and SETBP1 are shown as black bars, while integrations into non CIS-related genes are shown in grey. The insertions are listed according to their location within the affected gene expressed as the percentage of the overall length of the gene. The last column summarizes all integrations up to 5 kb downstream of a gene. Up, upstream of the start of transcription; down, downstream of the RefSeq gene. TSS, transcription start site.

FIG. 9 shows a LAM-PCR band pattern analysis of peripheral blood leukocytes and sorted CD14+, CD15+, CD3+ and CD19+ cells (purity CD3+/CD19+, >98%) derived from patient P1 from 21 to 542 days post-transplantation after undergoing CGD gene therapy as described in Example 1. M, 100 bp ladder; -C, 100 ng non-transduced human genomic DNA; 3′ IC, 3′-LTR internal control.

FIG. 10 shows a LAM-PCR band pattern analysis of peripheral blood leukocytes and sorted CD14+, CD15+, CD3+ and CD19+ cells (purity CD3+/CD19+, >98%) derived from patient P2 from 24 to 343 days post-transplantation after undergoing CGD gene therapy as described in Example 1. M, 100 bp ladder; -C, 100 ng non-transduced human genomic DNA; 3′ IC, 3′-LTR internal control.

FIG. 11 is a DNA map showing retroviral insertion site (RIS) clusters in highly active clones with integrants in the MDS1/EVI-1 gene. The insertions are tightly clustered within relevant regulatory upstream portions of the gene locus. Grey dots indicate RIS derived from P1, while white squares indicate RIS from P2.

FIG. 12 is a DNA map showing RIS clusters in highly active clones with integrants in the PRDM16 gene. The insertions are tightly clustered within relevant regulatory upstream portions of the gene locus. Grey dots indicate RIS derived from P1, while white squares indicate RIS from P2.

FIG. 13 is a DNA map showing RIS clusters in highly active clones with integrants in the SETBP1 gene. The insertions are tightly clustered within relevant regulatory upstream portions of the gene locus. Grey dots indicate RIS derived from P1, while white squares indicate RIS from P2.

FIG. 14 shows the long term follow up of individual clones contributing to hematopoiesis at different time points after transplantation in patient P1. Each individual CIS related clone detected is represented by one line, with each column representing an individual sampling time point. Grey boxes represent the detection of a specific clone at a time point via LAM-PCR, tracking PCR, and/or quantitative-competitive (QC-) PCR. The white boxes indicate the lack of detection at that time point, indicating that the clone contributes no or few cells to the peripheral circulation. *, no LAM-PCR performed; §, no tracking PCR performed; #, no QC-PCR performed.

FIG. 15 shows the long term follow up of individual clones contributing to hematopoiesis at different time points after transplantation in patient P2. Each individual CIS related clone detected is represented by one line, with each column representing an individual sampling time point. Grey boxes represent the detection of a specific clone at a time point via LAM-PCR, tracking PCR, and/or quantitative-competitive (QC-) PCR. The white boxes indicate the lack of detection at that time point, indicating that the clone contributes no or few cells to the peripheral circulation. *, no LAM-PCR performed; §, no tracking PCR performed; #, no QC-PCR performed.

FIG. 16 is a graph showing the overall contribution of clones with insertions in or near the three CIS-related RefSeq genes compared to all RIS locations at different time points detected in patient P1 during long-term myelopoiesis after gene modification. The insertion frequencies at MDS1-EVI-1 (light gray), PRDM16 (dark gray) and SETBP1 (black) in relation to non-CIS-related insertion frequencies (white) is illustrated as a percentage of all integration site junction sequences (entire column) detected at each specific time point. The black line denotes the approximate percentage of gene marked cells containing vector gp91^{phox} among peripheral blood granulocytes. BM, bone marrow; G, granulocytes; MC, monocytes; PB, peripheral blood.

FIG. 17 is a graph showing the overall contribution of clones with insertions in or near the three CIS-related RefSeq genes compared to all RIS locations at different time points detected in patient P2 during long-term myelopoiesis after gene modification. The insertion frequencies at MDS1-EVI-1 (light gray), PRDM16 (dark gray) and SETBP1 (black) in relation to non-CIS-related insertion frequencies (white) is illustrated as a percentage of all integration site junction sequences (entire column) detected at each specific time point. The black line denotes the approximate percentage of gene marked cells containing vector gp91^{phox} among peripheral blood granulocytes. BM, bone marrow; G, granulocytes; MC, monocytes; PB, peripheral blood.

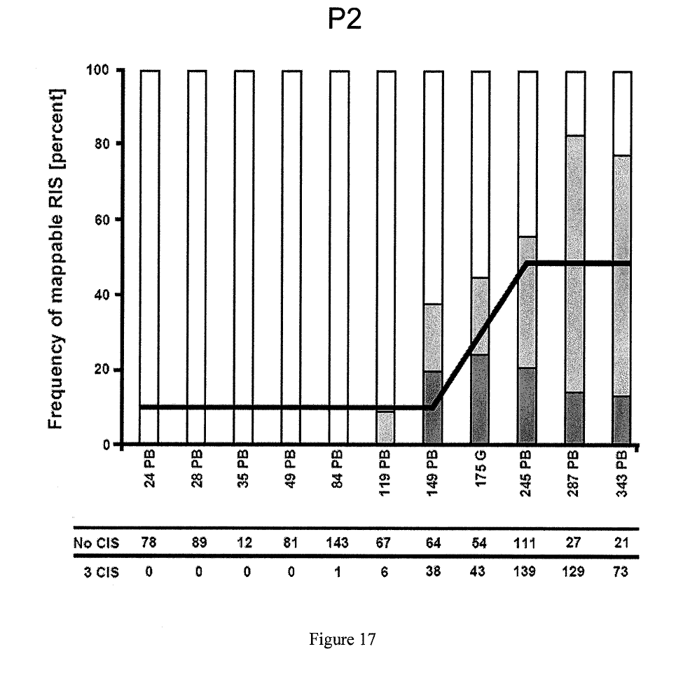

FIG. 18 illustrates a series of electrophoretic separations of nucleic acid on agarose gels showing the quantitative-competitive analysis of predominant clones from patient P1. The coamplification of 50 ng wild-type (WT) DNA from PB in competition with 500 copies of a 26-bp deleted internal standard (IS) allows semi-quantitative estimation of single clones. Time-course analysis revealed the sustained presence of all clones after their first detection (>3 months post-transplant). Numbers indicate days after transplantation. -C, 50 ng non-transduced genomic DNA.

FIG. 19 illustrates a series of electrophoretic separations of nucleic acid on agarose gels showing the quantitative-competitive analysis of predominant clones from patient P2. The coamplification of 50 ng wild-type (WT) DNA from PB in competition with 500 copies of a 26-bp deleted internal standard (IS) allows semi-quantitative estimation of single clones. Time-course analysis revealed the sustained presence of all clones after their first detection (>3 months post-transplant). Numbers indicate days after transplantation. -C, 50 ng non-transduced genomic DNA.

FIG. 20 shows LAM-PCR analysis of bone-marrow derived colonies from patient P1 at days +192 and +381 after transplantation. Colony numbers 1-3, 5, 7, 9-11, and 13 are colony-forming units-granulocyte-macrophage (CFU-GM)-derived colonies, whereas colonies 4, 6, 8, and 12 represent burst-forming units-erythrocyte (BFU-E) colonies. M, 100 bp ladder; -C, 100 ng nontransduced human genomic DNA.

FIG. 21 shows LAM-PCR analysis of bone-marrow derived colonies from patient P2 at day +245 after transplantation. Colony numbers 1-3 and 5 are CFU-GM-derived colonies, whereas colonies 4 and 6 represent BFU-E colonies. M, 100 bp ladder; -C, 100 ng nontransduced human genomic DNA.

FIG. 22 illustrates transcriptional activation of CIS genes by retroviral insertion. RT-PCR analysis of MDS1/EVI-1 (a), PRDM16 (b) and SETBP1 (c) was performed on bone marrow from patient P1 at day +381 and on peripheral blood leukocytes from patient P2 at days +287 and +343. Panel (a) shows analysis of MDS1/EVI-1 plus EVI-1 transcripts in the upper panel (PR+/PR−) and analysis of MDS1/EVI-1 only transcripts in the lower panel (PR+). The primer pairs used to detected EVI-1 transcripts are located within EVI-1 (exon 5 to exon 6) and therefore also detect MDS1/EVI-1 transcripts. In contrast, MDS/EVI-1 transcripts were detected with primer pairs located in the second exon of MDS1 and EVI-1 (Example 6). Panel (b) shows analysis of PR+/PR− in the upper panel and analysis of PR+ in the lower panel for PRDM16 transcripts. Panel (c) illustrates analysis of SETBP1 expression level. Panel (d) shows results from the β-actin RT-PCR. -C, H₂O control; PR, PR-domain; BM, bone marrow cells; PB, peripheral blood leukocytes; ND, healthy donor.

FIG. 23 illustrates expression of gp91^{phox} protein on transduced cells in the days after transplantation of the gene-modified cells. Granulocytes (CD15+) and T cells (CD3+) of patients P1 (a) and P2 (b) were labeled with the monoclonal antibody 7D5 and a lineage specific marker.

FIG. 24 show results that demonstrate continued expression of gp91^{phox} protein and functional reconstitution of NADPH oxidase activity in transduced cells. The top panel illustrates gp91^{phox} expression in CD34+ bone marrow cells of patient P1 at day +381. The bottom panel exhibits dithionite reduced minus oxidized differential spectra of flavocytochrome in protein extracts obtained from granulocytes. The granulocytes were isolated from the peripheral blood of a healthy donor (“control”), patient P1 at day +242 (“P1”) and patient P2 at day +120 (“P2”) after reinfusion of gene transduced cells. Granulocytes were also obtained from an X-CGD patient (“X-CGD”) for comparison. The two major absorption peaks at 426 nm (γ-peak) and 559 nm (α-peak) correspond to the reduced heme groups within gp91^{phox} and are visible in granulocyte extracts from a healthy donor and P1, while these bands are completely absent in extracts obtained from cells of an untreated X-CGD patient.

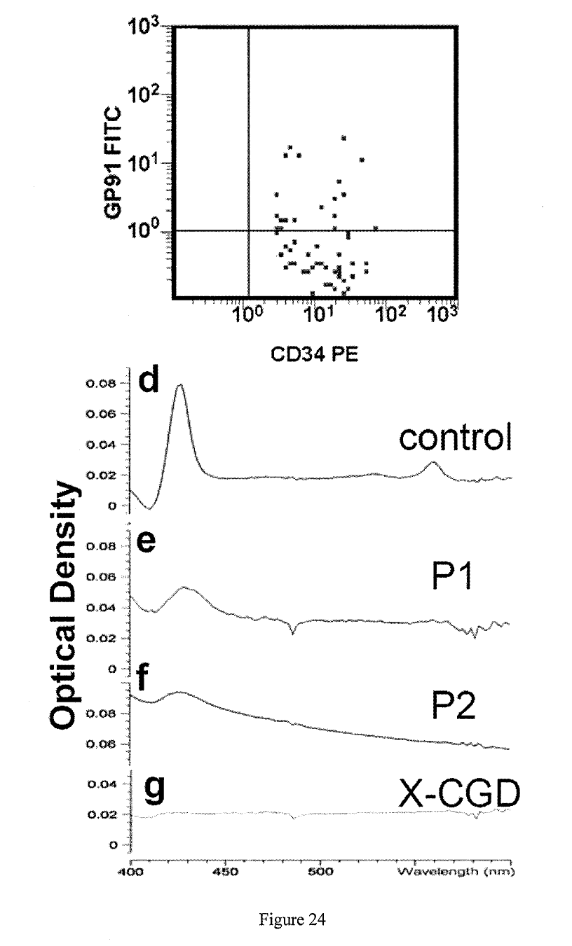

FIG. 25 illustrates functional reconstitution of NADPH oxidase activity in peripheral blood leukocytes (PBLs) and isolated granulocytes of patient P1 as revealed by oxidation of dihydrorhodamine (DHR) 123 and NBT reduction. Superoxide production in PBLs was measured by DHR 123 oxidation in opsonised E. coli, as indicated by black dots. Superoxide production in isolated granulocytes was measured by stimulation with PMA (open dots) or by reduction of NBT to formazan (open squares).

FIG. 26 shows an example of DHR 123 oxidation by neutrophils of patient P1 at day +473 after gene therapy both before (left panel) and after (right panel) PMA stimulation.

FIG. 27 shows NBT reduction in single granulocytes obtained from patient P1 at day +381 after gene therapy both before (left panel) and after (right panel) stimulation with opsonised zymosan (OPZ).

FIG. 28 illustrates functional reconstitution of NADPH oxidase activity in peripheral blood leukocytes (PBLs) and isolated granulocytes of patient P2 as revealed by oxidation of dihydrorhodamine (DHR) 123 and NBT reduction. Superoxide production in PBLs was measured by DHR 123 oxidation in opsonised E. coli, as indicated by black dots. Superoxide production in isolated granulocytes was measured by stimulation with PMA (open dots) or by reduction of NBT to formazan (open squares).

FIG. 29 shows an example of DHR 123 oxidation by neutrophils of patient P2 at day +344 after gene therapy both before (left panel) and after (right panel) PMA stimulation.

FIG. 30 shows NBT reduction in single granulocytes obtained from patient P2 at day +245 after gene therapy both before (left panel) and after (right panel) stimulation with opsonised zymosan (OPZ).

FIG. 31 illustrates superoxide anion production by granulocytes obtained from a healthy control (a), patient P1 at day +193 (b) and patient P2 at day +50 (c) as revealed by cytochrome c reduction after stimulation with 0.1 μg/ml PMA plus 1 μM fMLP. The reaction was inhibited by superoxide dismutase (SOD) or specific inhibitors of the phagocytic NADPH oxidase activity, such as 4-2-Aminoethylbenzene sulfonylfluoride (AEBSF) or diphenylene iodonium (DPI). In panel (a), 1×10⁶ cells/ml were used in the reaction, while in panels (b) and (c) 5×10⁶ cells/ml were used.

FIG. 32 shows the kinetics of E. coli killing by neutrophils obtained from a healthy donor (“pos. control”), patient P1 (“P1”), patient P2 (“P2”) and an individual with X-CGD (“X-CGD”) compared to incubation of E. coli in the absence of granulocytes as a negative control (“E. coli control”).

FIG. 33 illustrates transmission electron microscopy images of opsonised E. coli strain ML-35 at 2.5 hours after phagocytosis by granulocytes from the healthy donor (d, h), the X-CGD patient (b, e), and patient P1 at day +242 (c, f, g) at a ratio of 10:1 (E. coli:granulocytes). Black arrows in (e) and (f) denote undigested E. coli inside the phagocytic vacuole. White arrows in (g) and (h) indicate E. coli degradation. Inserts on the upper right hand corner show magnifications of undigested (e, f) and digested (g, h) bacteria. Encircled areas in panels (b-d) indicate enlarged cells shown in panels (e-h). Scale bars in panels (b-d) represent 5 μm; in panels (e-h), 2 μm.

FIG. 34 illustrates killing of A. fumigatus hyphae by gene-modified granulocytes as revealed by mitochondrial MTT reduction (a) and transmission electron microscopy (b-d). In panel (a), the time course of fungus killing is shown at a ratio of 1 seeded Aspergillus spore to 20 granulocytes obtained from either a healthy donor or patient P1 at day +381 after reinfusion of gene transduced cells. MTT reduction of Aspergillus hyphae alone was normalized to 100%. In panels (b-d), the fate of A. fumigatus hyphae after engulfment by healthy (b), non-corrected X-CGD (c) and functionally corrected (d) granulocytes is illustrated. Intact hyphae engulfed by phagocytes are marked with a black arrows (c, d), while hyphae with cytoplasmic disintegration entangled by phagocytes are marked with a white arrows (b, d). Bars in (b-d) represent 5 μm.

FIG. 35 shows fused PET scans of patient P1 (b) and fused PET-CT scans of patient P2 (c,d) both before (a,c) and either 50 (b) or 53 (d) daus after gene therapy. The circle in (a) denotes two active abscesses due to Staphylococcus aureus infection in the liver of patient P1, and the circle in (c) shows ¹⁸F-FDG uptake in the wall of a lung cavity of patient P2 due to A. fumigatus infection.

FIG. 36 shows that immortalized bone marrow cells (SF-1 cells) containing a Setbp1 integration can engraft and induce myeloid leukemia with minimal to mild maturation in irradiated transplanted mice. Immortalized clones usually appeared after 1 month of culturing. The figure shows gates for Ly5.1⁺ cells from bone marrow (a, left), spleen (b, left), and thymus (c, left) from a mouse (Ly5.2) 2 months after transplantation with the immortalized clone SF-1. Staining was done with Gr-1 (RB6-8C5)(a, right), CD19 (1D3)(b, right), and Thy-1.2 (53-2.1)(c, right) antibodies and corresponding isotype control antibodies (a-c, middle lane) in combination with Ly5.2 antibody. Numbers represent the percent of gated events. Details of this protocol are described in Du et al., Blood 106:3932-3939 (2005), herein incorporated by reference in its entirety.

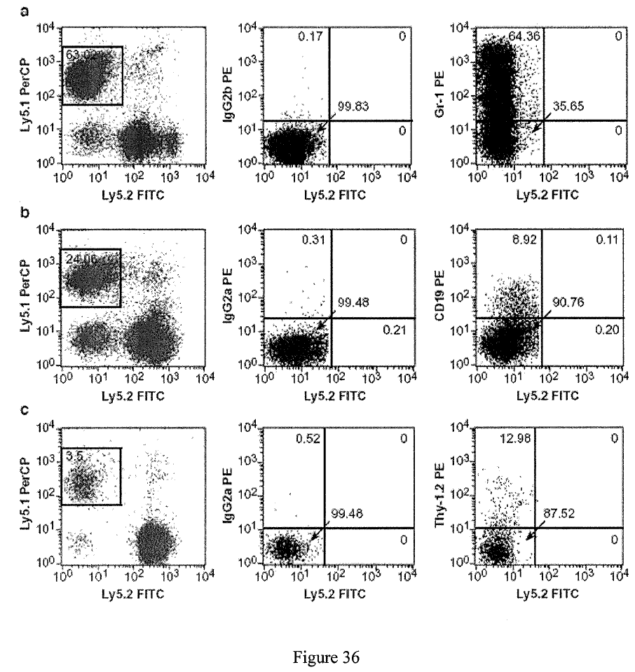

### BRIEF DESCRIPTION OF THE TABLES

Table 1 provides a list of proviral integration site sequences detected by LAM-PCR. LAM-PCR amplicons derived from patient P1 are shown in Table 1(a) while those from patient P2 are listed in Table 1(b). The RefSeq gene nearest to an identified integration site within a 100 kb window is listed. The two integrations in the most productive clone in patient P1 are defined by the “Sequence Identity” 77110 A09 (MDS1) and 75916 A08 (OSBPL6 and PRKRA). “Genomic Length” denotes the size of the LAM-PCR amplicon without linker- and LTR-sequences. “Sequence Orientation” denotes vector integration within the human genome. TSS, transcription start site; PB, peripheral blood; BM, bone marrow; CD15, purified granulocytes; CD14-15, monocytes; In, intron; Ex, exon.

Table 2 provides a list of vector integrants detected in the CIS genes MDS1/EVI-1, PRDM16 and SETBP1. Data for patient P1 is listed in Table 2(a) while data for patient P2 is listed in Table 2(b). Vector integration was detected by LAM-PCR (L), tracking PCR (T), and/or quantitative competitive PCR (Q). CIS clones chosen for a specific tracking over time are marked (T and/or Q) in the column “Track.” The most productive clone in P1 which was tracked using the sequence information obtained from 75916 A08 is annotated in this table by the second integration 77110 A09 (MDS1), which is also present in this particular clone. Empty spaces define no detection. CIS clones without “Integration Number” were additionally detected by tracking PCR due to their close location to other clones for which tracking PCR was performed. “Vector integration” indicates whether vector integration took place in the same orientation or in the reverse orientation of CIS gene expression. *, no LAM-PCR performed; §, no tracking PCR performed; #, no QC-PCR performed.

Table 3 provides a list of primers used for specific tracking of individual CIS clones and generation of clone specific internal standard. Flanking primers 1 and 2 (FP1 and FP2), in combination with vector specific primers, were used to track an individual CIS clone in patients P1 (Table 3a) and P2 (Table 3b) over time and to generate a clone specific internal standard. For quantitative competitive PCR vector specific primers and flanking primers 3 and 4 (FP3 and FP4) were used to coamplify a particular integration site and the appropriate internal standard (as described in Example 4).

Table 4 provides the accompanying SEQ ID NO for each primer listed in Table 3.

Table 5 is a summary of clinical data showing the colony formation of bone marrow total BM mononuclear cells obtained from bone marrow aspirates of patient P1.

Table 6 is a summary of clinical data showing the incorporation of 3H-Thymidine into mitogen- or antigen-stimulated mononuclear cells vs. non-stimulated mononuclear cells obtained from patients P1 and P2 at different time points.

Table 7 is a summary of clinical data showing examples of plasma protein levels at days +546 for patient P1 and day +489 for patient P2.

### DETAILED DESCRIPTION OF THE INVENTION

LAM-PCR analysis, described in U.S. Pat. No. 6,514,706, hereby incorporated by reference in its entirety, is a highly sensitive method for identifying an unknown nucleic acid sequence that flanks a known sequence present in a sample. The method is a powerful way to determine the insertion position of a transferred nucleic acid, such as a retroviral vector sequence, after an integration event. In addition to the use of LAM-PCR to determine target site selection of an integrated nucleic acid species, the method can also be used to determine how the integration sites change over time in a dividing cell culture. Thus, the method is particularly useful for clonal analysis of transfected hematopoietic cells or other transfected cells.

### CGD Patient Analysis Using LAM-PCR

LAM-PCR analysis was used to examine blood samples from two patients that were successfully receiving gene therapy by retroviral-based gene correction to treat chronic granulomatous disease (CGD) in an ongoing trial as described in Example 1. In the CGD gene therapy trial, high efficiency transduction of autologous CD34+ bone marrow cells and busulfan conditioning were used to successfully correct the cytochrome b gp91^{phox} gene defect in two patients for more than a year. A main goal of the analysis was to examine whether the retrovirus vector integration insertion site is less inert with respect to its genomic context than previously thought (Wu, et al. (2003) Science 300:1749-1751; Laufs, et al. (2003) Blood 101:2191-2198; Hematti, et al. (2004) PLoS Biol. 2:e423, each of which is hereby incorporated by reference in its entirety).

To determine whether an in vivo selective advantage of gene-modified myeloid cells capable of long term engraftment, proliferation and in vivo expansion, may be related to vector integration into particular genome regions, blood samples were taken from the two patients that achieved successful gene-corrected myelopoiesis in the CGD trial. A large-scale mapping analysis of retrovirus integration sites in the patient cells was then undertaken, using LAM-PCR as described in Example 3.

It was found that there is a significant influence of genomic vector integration on engraftment and proliferation of transduced hematopoietic cells. As shown herein, LAM-PCR based large-scale mapping of retrovirus integration sites (RIS) derived from the two successfully treated CGD patients shows that distribution of RIS became non-random starting about 3 months after reinfusion of gene corrected CD34+ cells.

The repopulating cell clones contained activating insertions in three genes. These three genes are the “positive regulatory (PR) domain” zinc finger genes MDS1/EVI-1 and PRDM16 and a SET binding protein SETBP1. The activating insertions were found to drive a 3 to 5 fold expansion of gene corrected cells, and selectively proliferated and dominated (>80%) gene-corrected long term myelopoiesis in both patients. These surprising results are in contrast to other research suggesting that retrovirus-based gene therapy would result in random monoallelic integration without relevant biological consequences (Coffin, et al. (1997), supra, which is hereby incorporated by reference in its entirety).

### EVI-1, PRDM16, and SETBP1

Two of the three genes that were found to contain the activating insertions encode zinc finger proteins that are related PR domain proteins. Several types of proteins, including certain transcriptional regulatory proteins, have regions that fold around a central zinc ion, producing a compact domain termed a “zinc finger.” Several classes of zinc-finger motifs have been identified. One group of zinc finger proteins is the “PR domain family” of transcription factor proteins, which includes, for example, the related genes EVI-1, PRDM16, and others. These PR domain family genes have been implicated, in some cases, to play a role in the development of cancer.

The EVI-1 protein (“ecotropic viral integration site 1”) is a zinc finger DNA-binding protein that is characterized by two domains of seven and three repeats of the Cys2-His2-type zinc finger motif (Morishita et al. (1988) Cell 54: 831-840; for a review, see Chi et al. (2003) J Biol Chem. 278:49806-49811, each of the foregoing which is hereby incorporated by reference in its entirety). Although EVI-1 is not generally detected in normal hematopoietic organs including bone marrow, the inappropriate expression of EVI-1 is often triggered by chromosomal rearrangements that disrupt the 3q26 chromosomal region where the EVI-1 gene is located (Fichelson, et al. (1992) Leukemia 6:93-99, which is hereby incorporated by reference in its entirety). Further, EVI-1 up-regulation can occur in chronic myelogenous leukemia patients, even though chromosomes appear normal by conventional cytogenetics, indicating that the inappropriate activation of EVI-1 can occur. High EVI-1 expression has been shown to predict poor survival in acute myeloid leukemia (Barjesteh van Waalwijk van Doom-Khosrovani, et al. (2003) Blood 101: 837-845, which is hereby incorporated by reference in its entirety). The related zinc finger protein PRDM16 (“positive regulatory domain containing 16”) has also been found to be a DNA binding protein.

The PR domain is characteristic for a sub-class of zinc finger genes that function as negative regulators of tumorigenesis [Fears, S. et al., 1996, Proc. Natl. Acad. Sci. 93:1642-1647, herein incorporated by reference in its entirety]. The PR domain of MDS1/EVI-1 (alias PRDM3) is a common target for wild-type retrovirus and vector insertion induced tumorigenesis, where the disruption of the PR domain activates PR domain negative oncogene EVI-1. Constitutive expression of the PR negative oncogene EVI-1 induces self-limiting myeloproliferation followed by a myelodysplastic syndrome in mice. The biology of PRDM16 (alias MDS1-EVI-1-like gene 1) is very similar to MDS1/EVI-1. In patients with myeloid malignancies, translocation of MDS1/EVI-1 or PRDM16 next to Ribophorin 1 gene on chromosome 3q21 leads to overexpression of the alternatively spliced PR domain negative transcript.

SET is a translocation breakpoint-encoded protein in acute undifferentiated leukaemia and SET binding protein 1 (SETBP1) is assumed to play a key role in SET associated leukemogenesis.

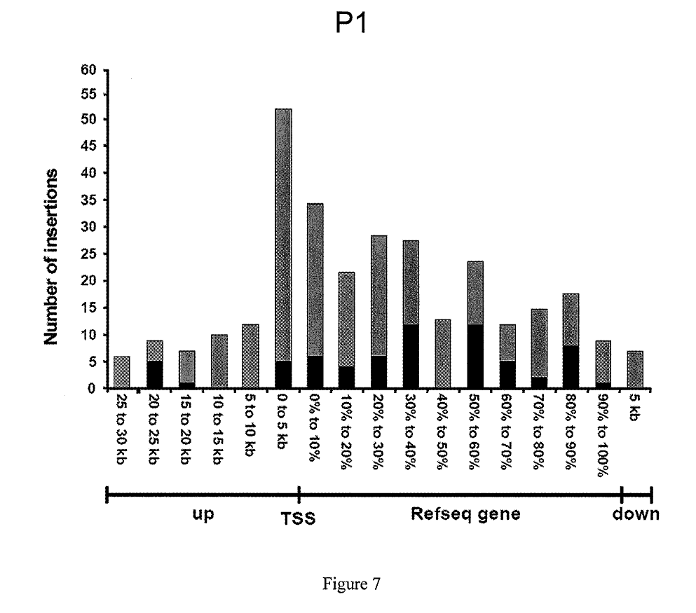

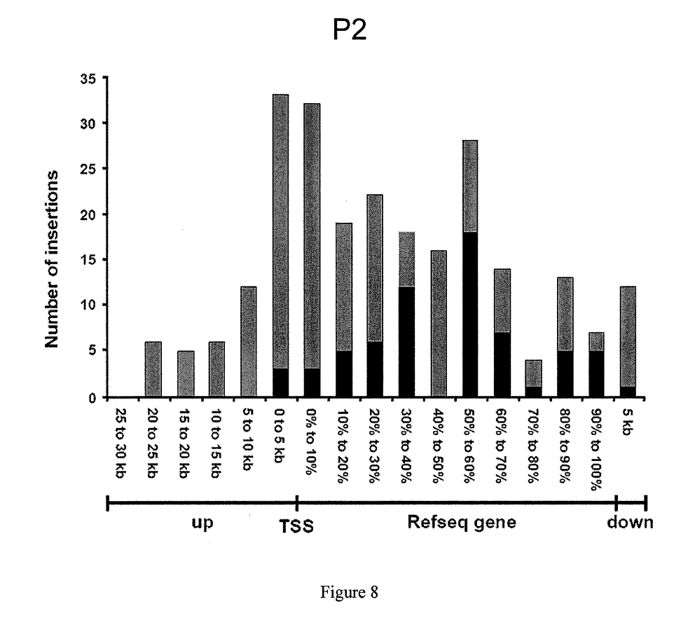

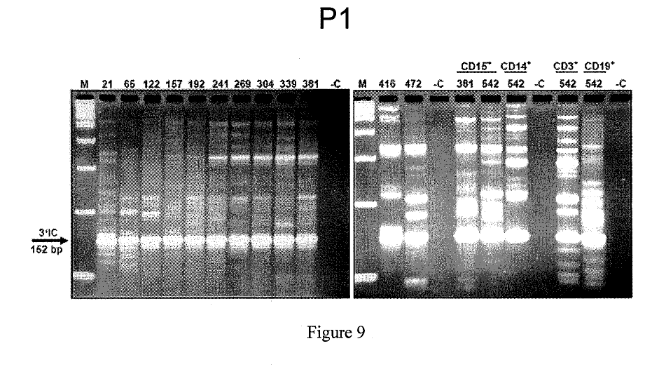

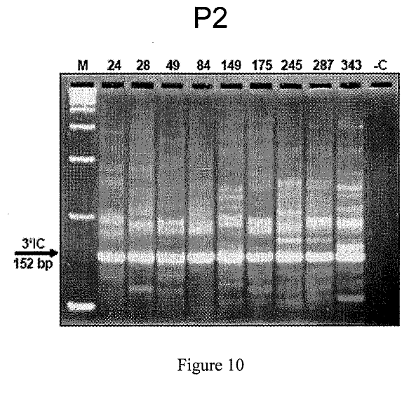

In experimental results, the LAM-PCR analysis showed a stable highly polyclonal hematopoietic repopulation of gene-corrected cells up to 381 days in patient 1 (P1) and up to 343 days in patient 2 (P2), although the band pattern indicated the appearance of individual pre-dominant clones 5 months after therapy (FIGS. 9, 10). A total of 948 unique RIS (patient P1: 551; patient P2: 397) were retrieved by shotgun cloning and sequencing of LAM products, of which 765 (P1: 435; P2: 330) could be mapped unequivocally to the human genome using the UCSC BLAT alignment tools. Integration preferentially occurred in gene coding regions (P1: 47%; P2: 52%) and was highly skewed to the ±5 kb transcriptional start site region (P1: 20%; P2: 21%) (FIGS. 7, 8).

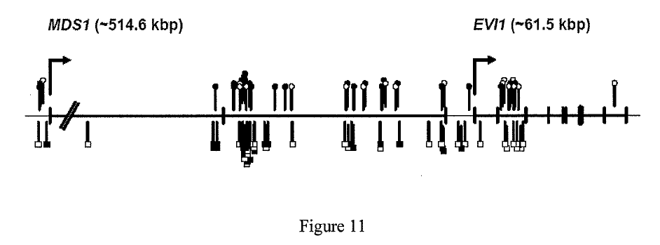

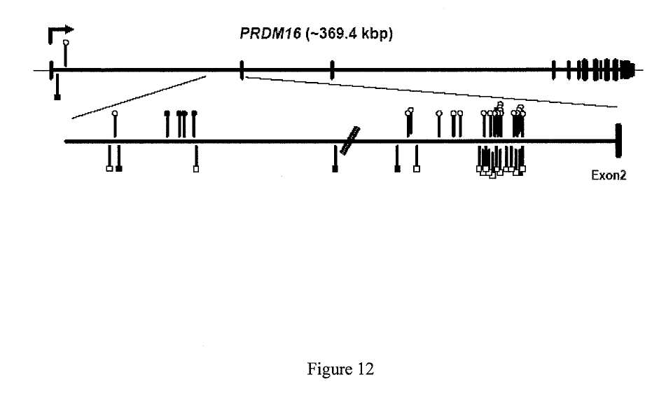

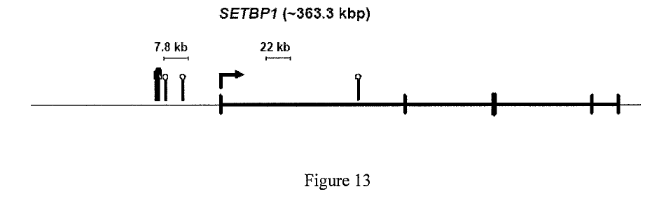

RIS distribution in both patients was not stable over time and became increasingly non-random but still polyclonal in both patients. The distribution also clustered to a much higher degree around particular common insertion sites (CIS) than shown by previous in vitro and in vivo integration site studies (Wu, et al. (2003) Science 300:1749-1751; Laufs, et al. (2003) Blood 101:2191-2198; and Hematti, et al. (2004) PLoS Biol. 2:e423, each of which is hereby incorporated by reference in its entirety). This clustering around common insertion sites allowed the prediction of the distribution and location of P2 insertions from the results in P1, whose gene modification procedure had been conducted 4 months earlier. The clonal contribution pattern turned into a less diverse pattern with distinct bands starting 5 months after therapy (FIGS. 9, 10), indicating the appearance of multiple predominant progenitor cell clones which subsequently contributed substantially to the proportion of gene-corrected granulocytes. Sequencing of insertion loci revealed that these pattern changes were due to the emergence of clones containing an insertion in one of 3 genetic loci, or CISs [Suzuki, T. et al. New genes involved in cancer identified by retroviral tagging. Nature Genet 32, 166-174 (2004), herein incorporated by reference in its entirety]. (Tables 1-3). All 134 detectable integrations at these three CISs occurred either in or near PR domain-containing zinc finger genes MDS1/EVI-1 or PRDM16 or in or near the SETBP1 gene. All insertions were located in or near the upstream region of these genes, preferentially close to the transcriptional start site or internal ATG sites (FIGS. 7, 8, and 11-13), exhibiting an unprecedented degree of non-random clustering.

Multiple clones with insertion sites in or near 2 particular positive regulatory (PR) domain zinc finger genes and SETBP1 began to emerge almost 3 months (patient P1: day 84; patient P2: day 80) after treatment, continuously developing to sustained clonal domination within the next 2 months after treatment (P1: day 157, P2: day 149) in both patients. Of 134 PR domain and SETBP1 CIS that have been detected, 91 distinct integrants were found in or near MDS1/EVI-1 (patient P1: 42; patient P2: 49), 36 in PRDM16 (P1: 18; P2: 18) and 7 in SETBP1 (P1: 7; P2: 0).

### Selective Advantage of EVI-1, PRDM16, and SETBP1 Integrants

Granulocytes have a life-span of 2-3 days. Therefore, the repeated detection over time of individual cell clones by retrovirus insertional marking is indicative of a repopulating progenitor cell or stem cell with long-term activity. The expansion of repopulating clones with these insertions occurred in both patients P1 and P2 with significant intensity. PR domain and SETBP1 related insertions comprised >90% of all clones detected at more than three time points after treatment. The in vivo selection advantage of these clones was further underlined by the observation that of 134 hits into gene loci affected by insertions more than three times, all of these CIS were related to these 3 genes. Within these gene loci, insertion events were highly non-randomly distributed and clustered near the transcriptional start site and internal ATG sites, strongly suggesting that a vector induced change of gene expression conferred a selective advantage to these clones (FIGS. 7,8, and 11-13).

In addition to the three genes discussed above, other gene insertion locations were found to be present. A summary list of the other LAM-PCR retrieved RIS and CIS is provided in Table 1.

TABLE 1a

| | | | | | | | | Upstream | In Gene, |
|---|---|---|---|---|---|---|---|---|---|
| Sequence | Days | | Genomic | Identity | | Sequence | Integration | of TSS | Distance to |
| Identity | Posttransplant | Sample | Length | [%] | Chromosome | Orientation | Locus | [bp] | TSS [bp] |
| 81519 G10 | 381 | PB | 90 | 100 | 1 | minus | 2851927 | | |
| 75916 B11 | 157 | CD15 | 119 | 100 | 1 | plus | 3018470 | | 9569 In1 |
| 75917 D12 | 192 | PB | 82 | 97.6 | 1 | plus | 3109854 | | 100953 In1 |
| 76778 G06 | 157 | CD15 | 93 | 99 | 1 | minus | 3110903 | | 102002 In1 |
| 76778 D03 | 157 | CD15 | 475 | 99.6 | 1 | minus | 3111126 | | 102225 In1 |
| 76777 C11 | 157 | PB | 363 | 99.8 | 1 | minus | 3111239 | | 102338 In1 |
| 76777 B04 | 192 | BM | 242 | 99.6 | 1 | minus | 3111424 | | 102523 In1 |
| 76778 G12 | 192 | BM | 163 | 100 | 1 | plus | 3122160 | | 103259 In1 |
| 77512 G08 | 241 | BM | 193 | 99 | 1 | plus | 3122190 | | 113289 In1 |
| 75523 G10 | 122 | PB | 58 | 100 | 1 | plus | 3123676 | | 114775 In1 |
| 76778 G04 | 157 | CD15 | 168 | 100 | 1 | plus | 3123793 | | 114892 In1 |
| 76774 E10 | 122 | PB | 26 | 100 | 1 | minus | 3123869 | | 114775 In1 |
| 75916 F03 | 157 | CD15 | 23 | 100 | 1 | plus | 3123915 | | 115014 In1 |
| 76777 B11 | 157 | PB | 324 | 99.7 | 1 | plus | 3123949 | | 115048 In1 |
| 75917 B07 | 192 | PB | 46 | 100 | 1 | plus | 3123975 | | 115074 In1 |
| 75917 G07 | 192 | BM | 267 | 100 | 1 | plus | 3124326 | | 115425 In1 |
| 76778 C05 | 157 | CD15 | 61 | 100 | 1 | plus | 3124344 | | 115443 In1 |
| 76778 B07 | 192 | PB | 108 | 100 | 1 | plus | 3124391 | | 115490 In1 |
| 78372 D05 | 269 | PB | 163 | 99.4 | 1 | plus | 3124446 | | 115545 In1 |
| 75921 B04 | 65 | PB | 65 | 98.5 | 1 | minus | 8392378 | | 419412 In13 |
| 90271 C12 | 542 | CD15 | 26 | 100 | 1 | minus | 11835171 | | 34611 Ex22 |
| 74718 D06 | 80 | PB | 551 | 99.7 | 1 | minus | 16320356 | 11604 | |
| 77051 E11 | 192 | BM | 38 | 100 | 1 | minus | 17376577 | 3421 | |
| 76778 C07 | 192 | PB | 588 | 100 | 1 | plus | 20669560 | | 8723 In1 |
| 75921 A01 | 21 | PB | 44 | 100 | 1 | minus | 26329358 | | 731 In1 |
| 75921 C08 | 80 | CD14-15 | 399 | 97.8 | 1 | plus | 32667914 | | 68163 In4 |
| 76778 B10 | 192 | PB | 66 | 100 | 1 | minus | 33125207 | | |
| 76777 D11 | 157 | PB | 310 | 99.7 | 1 | minus | 54271948 | | 40653 In4 |
| 75919 F11 | 80 | CD14-15 | 147 | 99.4 | 1 | minus | 54272035 | | 40740 In3 |
| 81507 A08 | 80 | PB | 53 | 98.2 | 1 | minus | 58762276 | 6810 | |
| 74718 E05 | 80 | PB | 31 | 100 | 1 | plus | 92552399 | | 13351 In10 |
| 74718 F06 | 80 | CD14-15 | 55 | 100 | 1 | plus | 111729279 | | 633 In1 |
| 87515 G01 | 381 | PB | 57 | 98.3 | 1 | minus | 112647073 | 3825 | |
| 81518 F05 | 381 | PB | 80 | 98.8 | 1 | minus | 120279248 | | 45070 In2 |
| 76771 F07 | 241 | BM | 29 | 100 | 1 | minus | 147492973 | | 13452 In9 |
| 77051 D06 | 192 | PB | 88 | 97.8 | 1 | plus | 153064832 | | 857 In1 |
| 75921 G06 | 80 | PB | 211 | 99.6 | 1 | minus | 161316286 | | 55691 In2 |
| 75916 F07 | 192 | BM | 193 | 99.5 | 1 | plus | 183498421 | 31341 | |
| 76771 G05 | 241 | PB | 185 | 100 | 1 | minus | 189822280 | 538 | |
| 81507 C11 | 80 | PB | 62 | 98.4 | 1 | plus | 195225630 | | 16102 In2 |
| 74718 H06 | 80 | CD14-15 | 51 | 100 | 1 | plus | 195311892 | 27990 | |
| 80484 E01 | 339 | PB | 73 | 100 | 1 | plus | 202405248 | | |
| 76777 D07 | 122 | PB | 49 | 100 | 1 | minus | 223433904 | 820 | |
| 82771 F11 | 416 | PB | 43 | 100 | 1 | minus | 231026099 | | |
| 75919 G11 | 80 | CD14-15 | 237 | 99.2 | 1 | plus | 231285099 | | |
| 76778 D09 | 192 | PB | 34 | 100 | 2 | plus | 8320708 | | 98439 In7 |
| 81947 H02 | 80 | PB | 51 | 100 | 2 | plus | 9771441 | 49737 | |
| 75523 A06 | 122 | PB | 41 | 97.6 | 2 | plus | 16434947 | | |
| 81947 F09 | 80 | PB | 91 | 100 | 2 | minus | 33633833 | | 60766 In2 |
| 80484 A02 | 339 | PB | 202 | 99.6 | 2 | plus | 45782505 | 8189 | |
| 75385 F05 | 80 | CD14-15 | 92 | 91 | 2 | minus | 64781672 | | |
| 81517 G07 | 381 | PB | 97 | 100 | 2 | minus | 71223208 | | |
| 75916 E08 | 157 | PB | 70 | 100 | 2 | plus | 87337038 | | |
| 78017 H03 | 269 | PB | 86 | 97.4 | 2 | plus | 89727409 | | |
| 81517 A05 | 381 | PB | 144 | 100 | 2 | minus | 160434956 | | 40439 In6 |
| 76062 G03 | 45 | PB | 345 | 97.1 | 2 | minus | 169753436 | | 6630 In3 |
| 75916 A08 | 157 | PB | 232 | 99.2 | 2 | minus | 179104254 | | |
| 77509 B01 | 241 | PB | 27 | 100 | 2 | plus | 179916869 | | 38136 In1 |
| 90271 A05 | 542 | CD15 | 35 | 100 | 2 | minus | 181999685 | | |
| 76774 B09 | 65 | PB | 58 | 100 | 2 | plus | 200428679 | | |
| 76062 C06 | 80 | PB | 58 | 94.3 | 2 | plus | 232926060 | | 274161 In9 |
| 76062 F05 | 80 | PB | 32 | 100 | 2 | plus | 237238212 | 22231 | |
| 75523 C12 | 122 | PB | 37 | 100 | 3 | plus | 17325010 | | 432393 In11 |
| 76062 B09 | 101 | PB | 152 | 98.5 | 3 | minus | 24315787 | | 195530 In2 |
| 75921 H05 | 80 | PB | 145 | 99.3 | 3 | minus | 48126921 | 21206 | |
| 75919 H11 | 80 | CD14-15 | 93 | 100 | 3 | plus | 50304093 | | 830 In1 |
| 78017 C08 | 269 | PB | 268 | 99.7 | 3 | minus | 56764928 | | 46065 In2 |
| 78016 D09 | 269 | PB | 47 | 100 | 3 | minus | 71587687 | | 87711 In2 |
| 74718 H02 | 45 | PB | 107 | 100 | 3 | plus | 71712844 | 37446 | |
| 81518 D11 | 381 | PB | 130 | 100 | 3 | plus | 87928481 | | |
| 77109 F03 | 241 | PB | 85 | 98.9 | 3 | plus | 116064142 | | 284675 In3 |
| 81947 F11 | 80 | PB | 21 | 100 | 3 | minus | 116302763 | | 46054 In1 |
| 90189 F09 | 542 | CD19 | 68 | 100 | 3 | minus | 120523758 | | 27848 In1 |
| 78372 H06 | 269 | PB | 76 | 100 | 3 | minus | 123036989 | | 265 In1 |
| 77509 G05 | 241 | PB | 175 | 100 | 3 | plus | 128988399 | | 36000 In1 |
| 75523 D08 | 21 | PB | 48 | 98 | 3 | plus | 130780456 | | 27903 In8 |
| 90189 H04 | 542 | CD19 | 36 | 100 | 3 | minus | 132590236 | | |
| 75921 G03 | 45 | PB | 74 | 100 | 3 | plus | 144803318 | | 246669 In6 |
| 75523 G05 | 122 | PB | 56 | 100 | 3 | minus | 151836834 | | |
| 80484 C04 | 339 | PB | 192 | 99.5 | 3 | minus | 158374771 | 13587 | |
| 75919 H12 | 122 | PB | 46 | 100 | 3 | minus | 167361204 | | |
| 81946 E12 | 80 | PB | 34 | 100 | 3 | plus | 169792527 | | |
| 77048 G07 | 241 | PB | 37 | 100 | 3 | minus | 170308560 | | 38235 In8 |
| 76771 H02 | 241 | PB | 153 | 98.7 | 3 | minus | 170337950 | | 8845 In2 |
| 77110 H11 | 241 | BM | 262 | 100 | 3 | plus | 170338708 | | 8087 In2 |
| 77110 D02 | 241 | BM | 25 | 100 | 3 | minus | 170339175 | | 7620 In2 |
| 75916 D12 | 192 | PB | 36 | 100 | 3 | minus | 170339748 | | 7047 In2 |
| 77048 E02 | 241 | PB | 60 | 100 | 3 | plus | 170340583 | | 6212 In2 |
| 76776 C04 | 157 | PB | 76 | 100 | 3 | minus | 170340730 | | 6065 In2 |
| 75917 C09 | 157 | PB | 99 | 100 | 3 | minus | 170342916 | | 3879 In2 |
| 75916 F04 | 192 | PB | 121 | 98.4 | 3 | minus | 170343812 | | 2983 In2 |
| 81520 F05 | 381 | PB | 209 | 100 | 3 | plus | 170344041 | | 2754 In2 |
| 75918 G04 | 192 | BM | 103 | 100 | 3 | plus | 170347592 | 797 | |
| 79207 B11 | 304 | PB | 58 | 100 | 3 | minus | 170350543 | 3748 | |
| 76776 G04 | 157 | PB | 70 | 100 | 3 | plus | 170351592 | | 512584 In2 |
| 81520 F05 | 381 | PB | 123 | 100 | 3 | plus | 170399072 | | 465104 In2 |
| 77049 G11 | 241 | BM | 86 | 100 | 3 | minus | 170400813 | | 463363 In2 |
| 76776 E04 | 157 | PB | 81 | 98.8 | 3 | minus | 170411959 | | 452217 In2 |
| 89252 E08 | 192 | CFU-GM5 | 44 | 100 | 3 | plus | 170415162 | | 449014 In2 |
| 74718 H10 | 122 | PB | 43 | 100 | 3 | plus | 170415288 | | 448888 In2 |
| 76776 A10 | 192 | BM | 113 | 98.3 | 3 | plus | 170433035 | | 431141 In2 |
| 77509 A03 | 241 | PB | 205 | 99.6 | 3 | minus | 170434026 | | 430150 In2 |
| 76062 D09 | 101 | PB | 86 | 100 | 3 | plus | 170444844 | | 419332 In2 |
| 74718 A07 | 80 | CD14-15 | 41 | 100 | 3 | plus | 170451100 | | 413076 In2 |
| 76062 E05 | 80 | PB | 31 | 100 | 3 | minus | 170452341 | | 411835 In2 |
| 75916 A01 | 157 | PB | 115 | 100 | 3 | minus | 170509909 | | 354267 In2 |
| 75917 B04 | 157 | CD15 | 95 | 97.9 | 3 | plus | 170516385 | | 347791 In2 |
| 74718 G05 | 80 | CD14-15 | 46 | 100 | 3 | plus | 170526878 | | 337298 In2 |
| 76771 D05 | 241 | PB | 135 | 100 | 3 | plus | 170551923 | | 312253 In2 |
| 77110 A09 | 241 | BM | 33 | 100 | 3 | plus | 170553839 | | 310337 In2 |
| 77049 B02 | 241 | BM | 22 | 100 | 3 | minus | 170556473 | | 307703 In2 |
| 76776 A11 | 192 | BM | 113 | 98.3 | 3 | plus | 170556716 | | 307460 In2 |
| 75385 B05 | 80 | CD14-15 | 134 | 99.3 | 3 | plus | 170557515 | | 306661 In2 |
| 78016 F03 | 269 | PB | 186 | 100 | 3 | plus | 170557567 | | 306609 In2 |
| 78016 C11 | 269 | PB | 334 | 99.8 | 3 | plus | 170558780 | | 305396 In2 |
| 75917 H11 | 157 | CD15 | 23 | 100 | 3 | plus | 170562183 | | 301993 In2 |
| 75916 A05 | 192 | PB | 134 | 99.3 | 3 | minus | 170563940 | | 300236 In2 |
| 78372 E08 | 269 | PB | 297 | 99.7 | 3 | minus | 170563955 | | 300221 In2 |
| 77110 F02 | 241 | BM | 153 | 100 | 3 | plus | 170573011 | | 291165 In2 |
| 77109 E01 | 241 | PB | 225 | 99.2 | 3 | plus | 170573083 | | 291093 In2 |
| 76776 G11 | 192 | BM | 197 | 100 | 3 | plus | 170588924 | | 275252 In1 |
| 77048 C07 | 241 | PB | 28 | 100 | 3 | minus | 170865275 | 1099 | |
| 75523 E11 | 122 | PB | 27 | 100 | 3 | plus | 170868261 | 4085 | |
| 79208 F04 | 304 | PB | 29 | 100 | 3 | plus | 170868263 | 4087 | |
| 76777 H12 | 157 | PB | 330 | 99.7 | 3 | minus | 188945076 | | 1101 In1 |
| 76776 B08 | 192 | PB | 145 | 100 | 3 | plus | 195022473 | | |
| 76771 D04 | 241 | PB | 59 | 100 | 4 | plus | 10381369 | 18611 | |
| 76777 G01 | 21 | PB | 437 | 99.4 | 4 | minus | 13470898 | | |
| 76062 G02 | 45 | PB | 96 | 99 | 4 | minus | 26562843 | | 24261 In1 |
| 74718 D12 | 122 | PB | 120 | 100 | 4 | plus | 38001579 | | |
| 77051 G08 | 157 | PB | 167 | 99.5 | 4 | minus | 48055868 | | 56874 In2 |
| 76778 D08 | 192 | PB | 74 | 100 | 4 | plus | 48658214 | 15819 | |
| 75523 C09 | 65 | PB | 101 | 99.1 | 4 | minus | 65745126 | | |
| 77051 C12 | 122 | PB | 20 | 100 | 4 | plus | 68396121 | 497 | |
| 75916 F09 | 157 | PB | 49 | 100 | 4 | plus | 78260938 | | 32864 In1 |
| 76774 A10 | 122 | PB | 271 | 99.7 | 4 | plus | 80493823 | | |
| 76776 A08 | 192 | PB | 239 | 98.8 | 4 | minus | 82445745 | | 37649 In4 |
| 75921 E12 | 122 | PB | 55 | 98.2 | 4 | plus | 88218318 | 67001 | |
| 90189 A04 | 542 | CD19 | 37 | 100 | 4 | minus | 95620404 | | 801 In1 |
| 74718 H01 | 21 | PB | 81 | 100 | 4 | minus | 95628740 | 15772 | |
| 74718 H12 | 122 | PB | 44 | 97.8 | 4 | plus | 104038725 | | 616 In1 |
| 77048 C09 | 241 | PB | 75 | 100 | 4 | plus | 124730726 | | |
| 81507 F08 | 80 | PB | 113 | 99.1 | 4 | minus | 140836266 | 1103 | |
| 74718 A09 | 101 | G | 283 | 99 | 4 | plus | 141088879 | | 343959 In2 |
| 79274 D09 | 304 | PB | 219 | 100 | 4 | plus | 184123942 | | |
| 75921 H08 | 101 | PB | 235 | 99.6 | 4 | minus | 184696250 | 44688 | |
| 76776 C06 | 157 | CD15 | 76 | 100 | 4 | minus | 185734365 | | 36487 In1 |
| 76777 C06 | 21 | PB | 50 | 100 | 5 | minus | 452019 | | 94728 In4 |
| 76771 A02 | 241 | PB | 63 | 100 | 5 | minus | 6841127 | | |
| 76778 A06 | 157 | CD15 | 81 | 100 | 5 | plus | 10538687 | | |
| 75916 H11 | 157 | CD15 | 72 | 100 | 5 | minus | 18739639 | | |
| 81520 E07 | 381 | PB | 99 | 98 | 5 | plus | 40235261 | | |
| 76777 A03 | 65 | PB | 318 | 100 | 5 | plus | 43156203 | 5837 | |
| 75523 D07 | 21 | PB | 131 | 98.5 | 5 | minus | 61913425 | | 169074 In24 |
| 75917 A04 | 157 | CD15 | 95 | 100 | 5 | minus | 74394225 | 31745 | |
| 75921 H03 | 45 | PB | 320 | 100 | 5 | plus | 80754434 | | 3006 In1 |
| 75919 C10 | 45 | PB | 25 | 100 | 5 | minus | 83296986 | | 419381 In9 |
| 76776 G10 | 192 | BM | 35 | 100 | 5 | minus | 100254880 | | 11989 In2 |
| 81507 F03 | 80 | PB | 44 | 100 | 5 | minus | 102482734 | | 1005 In1 |
| 90189 A11 | 542 | CD19 | 107 | 100 | 5 | minus | 159852447 | | |
| 75921 E11 | 122 | PB | 48 | 100 | 5 | plus | 163274211 | | |
| 90187 F03 | 542 | CD3 | 189 | 99.5 | 5 | minus | 171471522 | | 76343 In4 |
| 76774 H08 | 65 | PB | 256 | 99.7 | 5 | plus | 180605561 | 2059 | |
| 75523 D12 | 122 | PB | 80 | 100 | 6 | minus | 11839497 | | 47555 In4 |
| 75921 A12 | 122 | PB | 468 | 99.6 | 6 | minus | 13142134 | | |
| 74718 A11 | 122 | PB | 66 | 100 | 6 | minus | 15407000 | | 52494 In1 |
| 75921 D06 | 80 | PB | 84 | 100 | 6 | plus | 15476719 | | 122213 In1 |
| 76062 F03 | 45 | PB | 278 | 98.4 | 6 | plus | 25015792 | 30230 | |
| 90187 B07 | 381 | CD3 | 318 | 99.7 | 6 | minus | 26472648 | 729 | |
| 75921 E07 | 80 | CD14-15 | 132 | 99.3 | 6 | plus | 30041440 | 9431 | |
| 75523 G04 | 122 | PB | 135 | 98.6 | 6 | minus | 34350867 | 26123 | |
| 75916 F01 | 157 | PB | 135 | 98.6 | 6 | plus | 34361110 | 36366 | |
| 75917 C02 | 157 | PB | 132 | 90.8 | 6 | minus | 34361506 | 25727 | |
| 77512 B06 | 241 | PB | 88 | 100 | 6 | minus | 42856658 | 34846 | |
| 89252 B10 | 192 | BFU-E6 | 358 | 99.8 | 6 | minus | 45572266 | | 74374 In4 |
| 75921 A09 | 101 | PB | 83 | 97.6 | 6 | minus | 76365792 | 3196 | |
| 75916 B01 | 157 | PB | 46 | 100 | 6 | plus | 90958984 | | 104198 In4 |
| 76062 B08 | 80 | CD14-15 | 29 | 100 | 6 | plus | 91051984 | | 11198 In1 |
| 75921 F07 | 80 | CD14-15 | 92 | 100 | 6 | minus | 91683885 | | |
| 87515 E03 | 381 | PB | 83 | 98.8 | 6 | minus | 133094789 | | 2806 In2 |
| 77049 A09 | 241 | BM | 226 | 99.6 | 6 | plus | 139390529 | 39438 | |
| 75921 E04 | 65 | PB | 282 | 100 | 7 | minus | 10518582 | | |
| 77049 G04 | 241 | BM | 78 | 100 | 7 | minus | 11550522 | | |
| 77110 H08 | 241 | BM | 50 | 100 | 7 | plus | 13092529 | | |
| 78017 D02 | 269 | PB | 114 | 100 | 7 | minus | 18233804 | 75362 | |
| 77509 D12 | 241 | BM | 103 | 100 | 7 | plus | 24721029 | | 71971 In1 |
| 76777 H11 | 157 | PB | 259 | 99.3 | 7 | plus | 26971870 | 2334 | |
| 78372 G08 | 269 | PB | 108 | 100 | 7 | plus | 71179000 | | 177697 In3 |
| 77509 D04 | 241 | PB | 138 | 100 | 7 | plus | 71919645 | | 34711 In1 |
| 76778 A01 | 157 | PB | 176 | 100 | 7 | minus | 74032175 | | 235 In1 |
| 81520 F03 | 381 | PB | 360 | 99.5 | 7 | plus | 74839920 | | 173010 In9 |
| 81518 G07 | 381 | PB | 124 | 99.2 | 7 | plus | 79519852 | | |
| 80484 C07 | 339 | PB | 34 | 100 | 7 | minus | 99241099 | 16771 | |
| 78372 E05 | 269 | PB | 131 | 99.3 | 7 | minus | 128384363 | | 5700 In1 |
| 81518 E10 | 381 | PB | 26 | 100 | 7 | minus | 130155121 | | |
| 76778 B05 | 157 | CD15 | 624 | 99.9 | 7 | plus | 134397417 | | 23431 In8 |
| 76774 D11 | 122 | PB | 115 | 99.2 | 7 | minus | 136066960 | 89908 | |
| 76777 C10 | 122 | PB | 85 | 100 | 7 | minus | 138237992 | | 13728 In1 |
| 77051 F04 | 157 | PB | 74 | 100 | 7 | minus | 149504452 | 1057 | |
| 76778 A07 | 192 | PB | 209 | 100 | 7 | plus | 150407072 | | 4396 In1 |
| 76778 E03 | 157 | CD15 | 113 | 98.9 | 8 | minus | 11844483 | 81439 | |
| 77509 C12 | 241 | BM | 117 | 98.3 | 8 | minus | 24279381 | | |
| 88516 C02 | 381 | PB | 92 | 98.8 | 8 | minus | 27296114 | | 71198 In1 |
| 76778 B06 | 157 | CD15 | 318 | 98.4 | 8 | plus | 56940491 | 14435 | |
| 75921 G07 | 80 | CD14-15 | 113 | 99.2 | 8 | minus | 64221435 | 22282 | |
| 77051 E04 | 192 | BM | 119 | 89.4 | 8 | plus | 97060661 | | |
| 90271 F07 | 542 | CD15 | 86 | 98 | 8 | plus | 111841449 | | |
| 76062 H07 | 80 | CD14-15 | 73 | 100 | 9 | minus | 2008106 | | 2764 In1 |
| 76774 H04 | 21 | PB | 41 | 97.6 | 9 | plus | 15630206 | | 87109 In7 |
| 77051 B10 | 65 | PB | 40 | 100 | 9 | plus | 35780644 | 1762 | |
| 78372 H02 | 269 | PB | 164 | 100 | 9 | minus | 70264927 | 5833 | |
| 75916 D02 | 157 | PB | 36 | 100 | 9 | plus | 72797721 | 198 | |
| 74718 F02 | 45 | PB | 33 | 100 | 9 | minus | 72842849 | 45326 | |
| 76778 A02 | 157 | PB | 102 | 99.1 | 9 | plus | 90991577 | | |
| 90189 F04 | 542 | CD19 | 139 | 97.2 | 9 | minus | 92939250 | | 1195 In1 |
| 75916 E11 | 157 | CD15 | 313 | 99.7 | 9 | plus | 94904781 | | 336232 In10 |
| 81518 A01 | 381 | PB | 286 | 99.4 | 9 | minus | 113187276 | 5155 | |
| 75523 E04 | 65 | PB | 192 | 99.5 | 9 | minus | 113729493 | | |
| 80484 H02 | 339 | PB | 74 | 95.8 | 9 | minus | 120949449 | | 19321 In5 |
| 76156 E08 | 192 | PB | 82 | 94 | 9 | minus | 121126777 | | 16816 In2 |
| 76777 E09 | 122 | PB | 172 | 99.5 | 9 | plus | 122830903 | | 48032 In4 |
| 75917 A09 | 157 | PB | 176 | 99.5 | 9 | minus | 127265818 | 397 | |
| 76774 F06 | 65 | PB | 135 | 99.3 | 10 | plus | 8142340 | | 5667 In3 |
| 76777 F10 | 122 | PB | 23 | 100 | 10 | minus | 11426900 | | |
| 76777 B06 | 21 | PB | 86 | 98.9 | 10 | minus | 12348986 | | |
| 75385 H07 | 101 | G | 77 | 97.5 | 10 | plus | 17589360 | | |
| 76774 E02 | 21 | PB | 164 | 100 | 10 | minus | 20059603 | 85775 | |
| 76062 F09 | 101 | PB | 258 | 98.1 | 10 | plus | 27571912 | 2194 | |
| 75918 C04 | 192 | BM | 153 | 100 | 10 | minus | 52503037 | 1262 | |
| 75523 G11 | 122 | PB | 91 | 99 | 10 | minus | 54559814 | | |
| 89252 G10 | 192 | CFU-GM5 | 28 | 100 | 10 | plus | 72673678 | | 31323 In1 |
| 90273 B07 | 472 | PB | 88 | 100 | 10 | minus | 74067015 | 11172 | |
| 76062 D04 | 65 | PB | 200 | 100 | 10 | plus | 76380021 | | 111557 In1 |
| 75916 C06 | 192 | PB | 59 | 100 | 10 | plus | 80160285 | | |
| 76771 F05 | 241 | PB | 116 | 99.2 | 10 | minus | 91141817 | 541 | |
| 76777 F11 | 157 | PB | 111 | 99.1 | 10 | minus | 96964035 | | 20088 In5 |
| 75921 D05 | 65 | PB | 105 | 100 | 10 | minus | 104183329 | 986 | |
| 75916 | 157 | CD15 | 62 | 98.4 | 10 | plus | 116571061 | | |
| B04a | | | | | | | | | |
| 90189 D09 | 542 | CD19 | 62 | 100 | 10 | minus | 118542360 | | |
| 77051 A08 | 21 | PB | 130 | 99.3 | 10 | minus | 120885939 | | 32338 In8 |
| 75385 A05 | 80 | CD14-15 | 180 | 97.8 | 10 | minus | 120955266 | 1927 | |
| 74718 F08 | 101 | PB | 85 | 100 | 11 | minus | 5825254 | 58632 | |
| 76774 C09 | 122 | PB | 94 | 100 | 11 | minus | 6721651 | | |
| 76777 F08 | 122 | PB | 47 | 100 | 11 | minus | 9437408 | 1681 | |
| 77051 D10 | 192 | PB | 232 | 98.3 | 11 | plus | 23185782 | | |
| 75919 E12 | 101 | G | 26 | 100 | 11 | minus | 30458616 | | 100000 In3 |
| 81946 A01 | 80 | PB | 23 | 100 | 11 | minus | 33849881 | | 20531 In2 |
| 75917 D11 | 157 | CD15 | 150 | 98.7 | 11 | plus | 33909490 | 39078 | |
| 82772 A12 | 416 | PB | 22 | 100 | 11 | minus | 39701105 | | |
| 81519 H05 | 381 | PB | 82 | 100 | 11 | plus | 40086422 | | |
| 79207 C02 | 313 | PB | 313 | 100 | 11 | minus | 46322950 | | 11635 In1 |
| 75918 H03 | 192 | BM | 52 | 98.1 | 11 | minus | 47243007 | | 5698 In6 |
| 75921 F01 | 21 | PB | 341 | 99.2 | 11 | plus | 47556962 | | 139 Ex1 |
| 75916 C10 | 157 | CD15 | 403 | 99.6 | 11 | plus | 47566009 | | |
| 76778 C02 | 157 | PB | 169 | 98.9 | 11 | minus | 61967444 | | 103403 In4 |
| 75917 A03 | 157 | CD15 | 26 | 100 | 11 | minus | 74769215 | 28916 | |
| 75921 H06 | 80 | PB | 44 | 100 | 11 | plus | 77949261 | | 14106 In4 |
| 86978 A03 | 472 | PB | 29 | 100 | 11 | minus | 88030287 | | 390551 In2 |
| 76776 G08 | 192 | PB | 122 | 100 | 11 | plus | 93878516 | | |
| 75919 B10 | 45 | PB | 90 | 100 | 11 | plus | 95580187 | | 135805 In1 |
| 81517 H07 | 381 | PB | 45 | 100 | 11 | minus | 97653472 | | |
| 76062 G05 | 80 | PB | 35 | 100 | 11 | minus | 97672844 | | |
| 76778 C01 | 157 | PB | 102 | 100 | 11 | plus | 117627904 | | 317 In1 |
| 81947 E06 | 80 | PB | 31 | 100 | 11 | minus | 127927955 | 30584 | |
| 90189 C08 | 542 | CD19 | 45 | 97.8 | 11 | plus | 128095160 | | 25961 In1 |
| 75523 G02 | 21 | PB | 131 | 100 | 11 | plus | 129691058 | 1527 | |
| 90188 H01 | 381 | CD15 | 172 | 100 | 12 | minus | 612460 | | 30556 In1 |
| 74718 E01 | 21 | PB | 95 | 100 | 12 | minus | 1183131 | | 212466 In5 |
| 75385 H01 | 21 | PB | 467 | 99.4 | 12 | plus | 1899291 | 1160 | |
| 75916 C01 | 157 | PB | 142 | 99.3 | 12 | minus | 6592097 | 5360 | |
| 74718 C04 | 65 | PB | 121 | 99.2 | 12 | minus | 15742203 | | 91386 In1 |
| 82771 C10 | 416 | PB | 79 | 100 | 12 | minus | 24994257 | 758 | |
| 75916 H01 | 157 | PB | 115 | 98.3 | 12 | plus | 25096917 | | 409 Ex1 |
| 76776 F03 | 157 | PB | 216 | 99.6 | 12 | minus | 53647844 | 52045 | |
| 74718 E10 | 101 | G | 62 | 100 | 12 | plus | 53648119 | 51770 | |
| 74718 C08 | 101 | PB | 328 | 99.7 | 12 | minus | 53648489 | 51400 | |
| 75917 D08 | 192 | BM | 138 | 100 | 12 | minus | 54902894 | 923 | |
| 81519 A11 | 381 | PB | 75 | 100 | 12 | minus | 60709677 | | 163141 In1 |
| 80484 E03 | 339 | PB | 22 | 100 | 12 | plus | 61411185 | | |
| 75917 C04 | 157 | CD15 | 181 | 98.9 | 12 | minus | 63299271 | | 8711 In1 |
| 75921 H01 | 21 | PB | 65 | 100 | 12 | minus | 83874219 | | |
| 81519 E07 | 381 | PB | 53 | 100 | 12 | minus | 84728547 | | 4004 In1 |
| 75385 E07 | 101 | G | 64 | 98.5 | 12 | plus | 93103765 | | 58798 In4 |
| 79274 B02 | 304 | PB | 244 | 99.6 | 12 | plus | 93900671 | 33094 | |
| 88516 A04 | 381 | PB | 71 | 100 | 12 | minus | 94999380 | 67547 | |
| 74718 C01 | 21 | PB | 35 | 100 | 12 | plus | 100676875 | | 50225 In5 |
| 90189 A03 | 542 | CD19 | 52 | 100 | 12 | minus | 100676910 | | 50190 In5 |
| 75921 D12 | 122 | PB | 389 | 99.8 | 12 | plus | 108980608 | 12388 | |
| 75523 A11 | 122 | PB | 66 | 100 | 12 | plus | 117227962 | 45309 | |
| 76777 D01 | 21 | PB | 130 | 100 | 12 | minus | 122282159 | 592 | |
| 77048 H03 | 241 | PB | 32 | 100 | 12 | minus | 126568388 | | |
| 78017 G01 | 269 | PB | 93 | 99 | 12 | minus | 130229338 | | |
| 90189 A06 | 542 | CD19 | 57 | 98.3 | 13 | plus | 48765373 | | 45269 Ex10 |
| 74718 G08 | 101 | PB | 128 | 100 | 13 | minus | 66708923 | 6459 | |
| 76776 F07 | 192 | PB | 206 | 98.6 | 13 | plus | 88625758 | | |
| 81520 D02 | 381 | PB | 209 | 99.6 | 13 | minus | 90720133 | 77942 | |
| 82772 A09 | 416 | PB | 483 | 99.8 | 13 | plus | 98807517 | | 156353 In5 |
| 76777 F05 | 21 | PB | 161 | 100 | 14 | plus | 21612757 | | |
| 76062 D06 | 80 | PB | 309 | 99.7 | 14 | minus | 22135323 | 1663 | |
| 75917 A01 | 157 | PB | 39 | 100 | 14 | minus | 30566250 | | 1606 In1 |
| 81947 A05 | 80 | PB | 145 | 99.4 | 14 | minus | 33476777 | | 13260 In1 |
| 82773 F09 | 416 | PB | 111 | 99.1 | 14 | minus | 34829380 | 1945 | |
| 75523 E08 | 65 | PB | 43 | 100 | 14 | minus | 38773764 | | 888 In1 |
| 76776 B02 | 122 | PB | 29 | 100 | 14 | minus | 49121821 | | 1023 In2 |
| 81507 C04 | 80 | PB | 190 | 100 | 14 | minus | 51363193 | 50867 | |
| 76776 C01 | 122 | PB | 147 | 100 | 14 | plus | 52243339 | 329 | |
| 90271 H06 | 542 | CD15 | 91 | 100 | 14 | minus | 57963913 | | 6 Ex1 |
| 75916 D11 | 157 | CD15 | 85 | 100 | 14 | plus | 59104173 | | |
| 76777 A08 | 122 | PB | 29 | 100 | 14 | plus | 70195300 | 58163 | |
| 76777 C02 | 65 | PB | 352 | 99.8 | 14 | plus | 73307729 | 10984 | |
| 76778 D01 | 157 | PB | 106 | 100 | 14 | minus | 75687632 | 380 | |
| 81518 A06 | 381 | PB | 117 | 98.3 | 14 | minus | 90600116 | 3370 | |
| 74718 C05 | 80 | PB | 253 | 99.1 | 14 | minus | 106249125 | | |
| 75921 B12 | 122 | PB | 86 | 100 | 15 | minus | 30660905 | 34078 | |
| 80484 F05 | 339 | PB | 95 | 99 | 15 | plus | 38314376 | 4992 | |
| 75523 D06 | 122 | PB | 68 | 100 | 15 | minus | 42803694 | | |
| 76062 F07 | 80 | CD14-15 | 119 | 100 | 15 | plus | 48190860 | | 7851 In1 |
| 90189 C12 | 542 | CD19 | 30 | 96.7 | 15 | minus | 62087443 | | 38131 In2 |
| 76062 H02 | 45 | PB | 44 | 97.8 | 15 | plus | 62534868 | | |
| 76777 A12 | 157 | PB | 121 | 99.2 | 15 | plus | 62582835 | | |
| 75385 F08 | 122 | PB | 54 | 98.2 | 15 | plus | 72868044 | | 6276 In1 |
| 76778 H02 | 157 | PB | 131 | 100 | 15 | plus | 72869243 | | 7475 In1 |
| 81518 A05 | 381 | PB | 104 | 100 | 15 | minus | 79119084 | | |
| 76062 F04 | 65 | PB | 409 | 98.6 | 15 | plus | 80096251 | | |
| 79274 B07 | 304 | PB | 251 | 98.9 | 15 | minus | 83903434 | | 178559 In5 |
| 77051 G04 | 157 | PB | 303 | 99.7 | 15 | plus | 86891288 | 400 | |
| 75917 B10 | 157 | CD15 | 75 | 100 | 15 | minus | 88409297 | | 63541 In1 |
| 76776 A09 | 192 | PB | 176 | 100 | 15 | minus | 89713355 | | |
| 76771 A01 | 241 | PB | 63 | 96.9 | 15 | minus | 99479149 | | |
| 75917 B03 | 157 | CD15 | 95 | 97.9 | 15 | minus | 99493792 | | |
| 76777 G02 | 65 | PB | 142 | 100 | 16 | plus | 2145157 | 643 | |
| 76778 B03 | 157 | CD15 | 161 | 100 | 16 | minus | 3103114 | | 507 In1 |
| 76771 B12 | 241 | BM | 126 | 99.3 | 16 | minus | 16078404 | | 127469 In15 |
| 74718 C07 | 80 | CD14-15 | 178 | 98.9 | 16 | plus | 20663140 | 2521 | |
| 79207 C11 | 304 | PB | 152 | 100 | 16 | minus | 27320047 | 1177 | |
| 76777 B03 | 65 | PB | 104 | 97.1 | 16 | plus | 29221866 | | |
| 76778 B02 | 157 | PB | 62 | 100 | 16 | plus | 30453966 | 271 | |
| 81520 H08 | 381 | PB | 157 | 100 | 16 | minus | 47213606 | 11985 | |
| 81507 A12 | 80 | PB | 40 | 100 | 16 | minus | 49275147 | 2433 | |
| 81520 C11 | 381 | PB | 108 | 78.4 | 16 | minus | 51802696 | | 55357 In2 |
| 76062 D03 | 45 | PB | 193 | 99.5 | 16 | plus | 56202680 | 8779 | |
| 76774 A02 | 21 | PB | 20 | 100 | 16 | minus | 56234356 | | 22897 In2 |
| 76774 G12 | 122 | PB | 86 | 100 | 16 | plus | 71468127 | | 171648 In4 |
| 78017 G07 | 269 | PB | 148 | 99.4 | 16 | minus | 72645953 | | |
| 77051 G06 | 157 | PB | 70 | 98.6 | 16 | plus | 73612301 | | 21885 In1 |
| 81947 C08 | 80 | PB | 22 | 95.5 | 16 | minus | 78195490 | 3378 | |
| 76776 H12 | 192 | BM | 173 | 93.8 | 16 | plus | 80228327 | | 191169 In3 |
| 75385 B02 | 45 | PB | 227 | 100 | 16 | minus | 83925895 | | |
| 77048 G02 | 241 | PB | 127 | 100 | 17 | minus | 2065718 | | 88051 In6 |
| 81507 C01 | 80 | PB | 150 | 100 | 17 | minus | 3089222 | | |
| 78017 B03 | 269 | PB | 31 | 96.8 | 17 | plus | 7472233 | 344 | |
| 89253 D10 | 381 | CFU-GM9 | 98 | 100 | 17 | minus | 15788426 | 530 | |
| 74718 F12 | 122 | PB | 258 | 98.9 | 17 | plus | 15810598 | | 21642 In1 |
| 75523 H11 | 122 | PB | 221 | 99.1 | 17 | plus | 16241490 | 18123 | |
| 75916 D01 | 157 | PB | 111 | 99.1 | 17 | plus | 26661380 | | 215137 In25 |
| 74718 B07 | 80 | CD14-15 | 142 | 99.3 | 17 | minus | 30442109 | 1702 | |
| 76777 D08 | 122 | PB | 79 | 100 | 17 | minus | 34087443 | 27969 | |
| 75921 E01 | 21 | PB | 534 | 100 | 17 | minus | 37683324 | 9767 | |
| 74718 B04 | 65 | PB | 57 | 100 | 17 | plus | 37728376 | | 65555 In13 |
| 75385 E05 | 80 | CD14-15 | 415 | 99.6 | 17 | minus | 43993964 | | 11718 In1 |
| 90273 H04 | 472 | PB | 116 | 100 | 17 | minus | 50632288 | | |
| 75523 E10 | 122 | PB | 132 | 100 | 17 | minus | 50759597 | | |
| 76776 D10 | 192 | BM | 39 | 97.5 | 17 | minus | 59107061 | | 53528 In6 |
| 76774 A06 | 65 | PB | 121 | 100 | 17 | plus | 59415018 | 11008 | |
| 75921 F02 | 45 | PB | 53 | 100 | 17 | plus | 64736203 | | 16348 In2 |
| 80484 A06 | 339 | PB | 68 | 100 | 18 | minus | 5506306 | | 27680 In1 |
| 76778 C09 | 192 | PB | 182 | 100 | 18 | minus | 7361344 | | |
| 76062 E09 | 101 | PB | 191 | 99.5 | 18 | minus | 13127536 | | |
| 90188 B01 | 381 | CD15 | 21 | 100 | 18 | plus | 21131910 | | 54204 In3 |
| 76776 D08 | 192 | PB | 140 | 99.3 | 18 | plus | 21166083 | | 20031 In1 |
| 76774 B12 | 122 | PB | 88 | 100 | 18 | minus | 27875268 | | 22825 In2 |
| 75523 B10 | 122 | PB | 175 | 100 | 18 | minus | 40340930 | | |
| 76778 G07 | 192 | PB | 53 | 98.2 | 18 | minus | 40513701 | 21766 | |
| 79274 B06 | 304 | BM | 46 | 100 | 18 | minus | 40513716 | 21751 | |
| 77512 B07 | 241 | BM | 31 | 96.8 | 18 | minus | 40513723 | 21744 | |
| 76778 F12 | 192 | BM | 81 | 86.5 | 18 | plus | 40513795 | 21672 | |
| 76776 E09 | 192 | PB | 105 | 99.1 | 18 | plus | 40513912 | 21555 | |
| 75916 G10 | 157 | CD15 | 100 | 100 | 18 | plus | 40517135 | 18332 | |
| 77509 D02 | 241 | PB | 146 | 98.7 | 18 | plus | 40661930 | | 126463 In1 |
| 76778 E06 | 157 | CD15 | 388 | 99.3 | 18 | minus | 44789903 | | |
| 75921 F04 | 65 | PB | 113 | 99.2 | 18 | minus | 61574589 | | 5452 In1 |
| 75917 F11 | 157 | CD15 | 36 | 100 | 18 | plus | 66543930 | | |
| 77109 G08 | 241 | PB | 183 | 99.5 | 18 | minus | 72903252 | 45290 | |
| 77109 C08 | 241 | PB | 133 | 100 | 18 | minus | 72903302 | 45340 | |
| 76777 G11 | 157 | PB | 66 | 100 | 18 | plus | 75369456 | | 108142 In9 |
| 77051 C08 | 122 | PB | 245 | 99.6 | 19 | plus | 1354011 | 1459 | |
| 75921 E06 | 80 | PB | 83 | 100 | 19 | minus | 2035016 | | 12253 In2 |
| 77051 A04 | 21 | PB | 222 | 99.6 | 19 | plus | 3292661 | 17955 | |
| 86978 G01 | 472 | PB | 212 | 100 | 19 | minus | 11301964 | | 9357 |
| 76774 C11 | 122 | PB | 331 | 99.7 | 19 | plus | 12908833 | 1590 | |
| 77051 C01 | 122 | PB | 120 | 98.4 | 19 | minus | 14500924 | 458 | |
| 76776 E10 | 192 | BM | 67 | 98.6 | 19 | minus | 41807119 | | 19058 In4 |
| 78372 H05 | 269 | PB | 83 | 100 | 19 | plus | 46020414 | | |
| 81519 H11 | 381 | PB | 343 | 95.1 | 19 | plus | 55064096 | | |
| 81507 F06 | 80 | PB | 91 | 100 | 19 | plus | 59589897 | 21617 | |
| 76777 G09 | 122 | PB | 76 | 98.7 | 19 | minus | 60750543 | | |
| 78372 C09 | 269 | PB | 31 | 100 | 20 | minus | 255449 | | 1210 Ex1 |
| 76156 C09 | 157 | PB | 50 | 100 | 20 | minus | 5007728 | | |
| 75917 H01 | 157 | PB | 80 | 98.8 | 20 | minus | 8179560 | | 118264 In2 |
| 77051 A03 | 21 | PB | 146 | 99.4 | 20 | plus | 17889438 | | 7716 In1 |
| 78017 G05 | 269 | PB | 319 | 99.1 | 20 | plus | 23083452 | | |
| 77051 A11 | 21 | PB | 49 | 100 | 20 | plus | 23289671 | 3350 | |
| 75921 | 45 | PB | 51 | 100 | 20 | minus | 30733487 | | |
| 78017 C11 | 269 | PB | 63 | 100 | 20 | plus | 31022967 | | |
| 78372 C06 | 269 | PB | 159 | 100 | 20 | minus | 34115747 | 48015 | |
| 76777 D02 | 65 | PB | 344 | 99.8 | 20 | minus | 45571075 | | 7011 In1 |
| 75917 B01 | 157 | PB | 127 | 96.1 | 20 | plus | 47353619 | 25610 | |
| 76062 G10 | 101 | G | 126 | 99.3 | 20 | minus | 61735412 | | |
| 78017 D06 | 269 | PB | 137 | 99.3 | 21 | plus | 20230740 | | |
| 83397 G03 | 339 | PB | 65 | 96.8 | 21 | minus | 26864102 | | 3350 In1 |
| 76774 B07 | 65 | PB | 144 | 100 | 21 | minus | 36444161 | | |
| 81507 A02 | 80 | PB | 48 | 100 | 21 | plus | 37650033 | 11696 | |
| 82771 E03 | 416 | PB | 79 | 100 | 21 | minus | 38679600 | | 112667 In10 |
| 76777 H06 | 21 | PB | 121 | 100 | 21 | minus | 44021236 | 549 | |
| 77509 F01 | 241 | PB | 89 | 98.9 | 22 | minus | 22512791 | 7036 | |
| 76774 H01 | 21 | PB | 39 | 97.5 | 22 | plus | 23236042 | | 20176 In6 |
| 90187 A06 | 381 | CD3 | 37 | 100 | 22 | minus | 26448144 | | |
| 81507 D03 | 80 | PB | 40 | 97.5 | 22 | plus | 26467846 | | |
| 76774 D10 | 122 | PB | 106 | 100 | 22 | plus | 26505858 | | 16182 In1 |
| 75523 B09 | 65 | PB | 76 | 100 | 22 | plus | 27530812 | 9698 | |
| 76774 B05 | 21 | PB | 213 | 98.2 | 22 | plus | 28274636 | 438 | |
| 79208 A01 | 304 | PB | 31 | 100 | 22 | plus | 29955672 | | 22868 In2 |
| 77051 C02 | 122 | PB | 80 | 100 | 22 | plus | 34630853 | 69925 | |
| 77512 E06 | 241 | PB | 471 | 99.2 | 22 | plus | 35050897 | | 57584 In3 |
| 76776 F10 | 192 | BM | 23 | 100 | 22 | plus | 35201686 | | 501 In1 |
| 75921 F12 | 122 | PB | 51 | 98.1 | 22 | plus | 36028183 | | 25259 In8 |
| 77051 B02 | 65 | PB | 41 | 100 | 22 | minus | 37474422 | | 2025 In1 |
| 90189 G10 | 542 | CD19 | 61 | 100 | X | plus | 11537921 | | 1986 In2 |
| 80484 D12 | 339 | PB | 45 | 92.4 | X | minus | 23291457 | | |
| 77109 C04 | 241 | PB | 29 | 100 | X | minus | 23728961 | | |
| 81517 F05 | 381 | PB | 190 | 97.9 | X | plus | 77715434 | | |
| 77048 H08 | 241 | PB | 83 | 98.8 | X | minus | 130848844 | 34018 | |
| 75916 B02 | 157 | PB | 142 | 100 | X | minus | 148303433 | 10881 | |
| 75523 B01 | 21 | PB | 176 | 100 | Y | plus | 13985233 | | 45448 In3 |
| 76774 B06 | 65 | PB | 21 | 100 | Y | plus | 21749914 | | |

| | Downstream | Next RefSeq | | |
|---|---|---|---|---|
| Sequence | of | Gene (within | | Additionally Detected at Days |
| Identity | Gene [bp] | 100 kb) | More RefSeq Genes within 100 kb | Posttransplant |
| 81519 G10 | | | no Refseq gene within next 100 kb | 542 CD14 |
| 75916 B11 | | PRDM16 | | 192 PB, 304 PB |
| 75917 D12 | | PRDM16 | | |
| 76778 G06 | | PRDM16 | | 542 CD15 |
| 76778 D03 | | PRDM16 | | |
| 76777 C11 | | PRDM16 | | |
| 76777 B04 | | PRDM16 | | 157 CD15 |
| 76778 G12 | | PRDM16 | | 157 PB |
| 77512 G08 | | PRDM16 | | |
| 75523 G10 | | PRDM16 | | 157 CD15, 192 BM, 241 PB |
| 76778 G04 | | PRDM16 | | 157 PB |
| 76774 E10 | | PRDM16 | | |
| 75916 F03 | | PRDM16 | | 157 PB and CD15, 241 BM |
| 76777 B11 | | PRDM16 | | 192 PB and BM, 241 BM, 304 PB |
| 75917 B07 | | PRDM16 | | 157 PB and CD15, 241 BM, 269 |
| | | | | PB |
| 75917 G07 | | PRDM16 | | 157 PB, 192 PB, 241 BM and PB |
| 76778 C05 | | PRDM16 | | 157 PB, 192 PB, 269 PB |
| 76778 B07 | | PRDM16 | | 157 PB, 269 PB, 304 PB |
| 78372 D05 | | PRDM16 | | |
| 75921 B04 | | RERE | 9619 bp upstream of | |
| | | | DKFZp566H0824 | |
| 90271 C12 | | CLCN6 | 4871 bp downstream of NPPA and | |
| | | | 16619 bp downstream of NPPB and | |
| | | | 34923 bp upstream of MTHFR and | |
| | | | 79218 bp downstream of KIAA2013 | |
| | | | and 90083 bp downstream of | |
| | | | AGTRAP and 93841 bp upstream of | |
| | | | PLOD1 | |
| 74718 D06 | | MGC10731 | | |
| 77051 E11 | | PADI4 | 2054 bp downstream of PADI3 | 304 PB |
| 76778 C07 | | CDA | 35694 bp upstream of PINK1 and | |
| | | | 42754 bp downstream of FAM43B | |
| | | | and 54017 bp downstream of DDOST | |
| | | | and 66255 bp downstream of KIF17 | |
| 75921 A01 | | CDW52 | 12103 bp upstream of SOC and | |
| | | | 3156 bp downstream of AIM1L | |
| 75921 C08 | | ZBTB8 | | |
| 76778 B10 | 18911 | AK2 | 26400 bp upstream of IBRDC3 and | |
| | | | 89161 bp upstream of BCLP and | |
| | | | 90600 bp upstream of ADC and | |
| | | | 95868 bp downstream of HPCA | |
| 76777 D11 | | FLJ32112 | 40816 bp upstream of C1orf8 | |
| 75919 F11 | | FLJ32112 | 40903 bp upstream of C1orf8 | |
| 81507 A08 | | TACSTD2 | 37809 bp upstream of OMA1 | |
| 74718 E05 | | FLJ13150 | | |
| 74718 F06 | | MGC24133 | | |
| 87515 G01 | | DKFZp547A023 | 75005 bp upstream of WNT2B | |
| 81518 F05 | | NOTCH2 | | 157 PB |
| 76771 F07 | | NOHMA | 10267 bp upstream of GPP34R and | |
| | | | 22651 bp downstream of CTSS and | |
| | | | 78291 bp upstream of ENSA and | |
| | | | 88787 bp downstream of CTSK | |
| 77051 D06 | | EST1B | 980 bp upstream of MGC13102 and | |
| | | | 10726 bp downstream of MGC31963 | |
| | | | and 16656 bp downstream of VHLL | |
| | | | and 33967 bp upstream of PAQR6 | |
| 75921 G06 | | PBX1 | | |
| 75916 F07 | | PLA2G4A | | |
| 76771 G05 | | HRPT2 | 16067 bp upstream of GLRX2 and | |
| | | | 35509 bp downstream of SSA2 and | |
| | | | 57553 bp upstream of B3GALT2 and | |
| | | | 62102 bp upstream of UCHL5 | |
| 81507 C11 | | ATP6V1G3 | | |
| 74718 H06 | | PTPRC | | |
| 80484 E01 | 13518 | NUCKS | 24018 bp upstream of PCANAP6 | |
| 76777 D07 | | CABC1 | 52884 bp downstream of CDC42BPA | |
| | | | and 43372 bp downstream of PSEN2 | |
| 82771 F11 | 23414 | IRF2BP2 | | |
| 75919 G11 | | | no Refseq gene within next 100 kb | |
| 76778 D09 | | LOC339789 | | |
| 81947 H02 | | YWHAQ | | |
| 75523 A06 | | | no Refseq gene within next 100 kb | |
| 81947 F09 | | RASGRP3 | 86547 bp downstream of | |
| | | | DKFZP564F0522 and 97609 bp | |
| | | | downstream of LTBP1 | |
| 80484 A02 | | PRKCE | 32451 bp upstream of FLJ10379 | 416 PB |
| 75385 F05 | 49885 | AFTIPHILIN | | |
| 81517 G07 | 5782 | NAGK | 25266 bp downstream of MCEE and | |
| | | | 45891 bp upstream of MPHOSPH10 | |
| | | | and 89552 bp upstream of TEX261 | |
| | | | and 99084 bp downstream of | |
| | | | FLJ12056 | |
| 75916 E08 | | | no Refseq gene within next 100 kb | |
| 78017 H03 | | | no Refseq gene within next 100 kb | |
| 81517 A05 | | MARCH7 | 15915 bp downstream of CD302 and | |
| | | | 50427 bp downstream of LY75 | |
| 76062 G03 | | DHRS9 | | |
| 75916 A08 | 17403 | PRKRA | 17874 bp downstream of OSBPL6 | 157CD15, 192 PB and BM and |
| | | | and 51278 bp downstream of FKBP7 | CFU-GM3, 122 PB, 241 PB and |
| | | | and 66613 bp upstream of PLEKHA3 | BM, 269 PB, 304 PB, 339 PB, 381 |
| | | | | PB and CD15 and CD3, 416 PB, |
| | | | | 472 PB, 542 CD14 and CD15 and |
| | | | | CD19 |
| 77509 B01 | | SESTD1 | | |
| 90271 A05 | | | no Refseq gene within next 100 kb | |
| 76774 B09 | | | no Refseq gene within next 100 kb | |
| 76062 C06 | | MGC42174 | | |
| 76062 F05 | | CMKOR1 | 40120 bp upstream of FLJ22527 | |
| 75523 C12 | | TBC1D5 | | |
| 76062 B09 | | THRB | | |
| 75921 H05 | | MAP4 | 46751 bp downstream of CDC25A | |
| 75919 H11 | | IFRD2 | 1173 bp downstream of HYAL3 and | |
| | | | 3545 bp downstream of FLJ38608 | |
| 78017 C08 | | ARHGEF3 | 91529 bp upstream of RAP140 | |
| 78016 D09 | | FOXP1 | | |
| 74718 H02 | | FOXP1 | | |
| 81518 D11 | | | no Refseq gene within next 100 kb | |
| 77109 F03 | | ZBTB20 | | |
| 81947 F11 | | ZBTB20 | | |
| 90189 F09 | | CDGAP | 81316 bp upstream of B4GALT4 | |
| 78372 H06 | | EAF2 | 373 bp upstream of IQCB1 and | |
| | | | 58988 bp upstream of SLC15A2 and | |
| | | | 85697 bp upstream of GOLGB1 | |
| 77509 G05 | | MGLL | 105939 bp downstream of ABTB1 | |
| 75523 D08 | | PLXND1 | | |
| 90189 H04 | 2609 | NUDT16 | 24167 bp downstream of LOC152195 | |
| | | | and 38235 bp downstream of NEK11 | |
| | | | and 73508 bp downstream of MRPL3 | |
| 75921 G03 | | SLC9A9 | | |
| 75523 G05 | 5904 | SELT | 23541 bp downstream of MGC39662 | 241 PB and BM, 269 PB |
| | | | and 52109 bp downstream of EIF2A | |
| | | | and 89899 bp upstream of SERP1 | |
| 80484 C04 | | CCNL1 | 86629 bp downstream of FLJ12604 | |
| 75919 H12 | | | no Refseq gene within next 100 kb | 241 PB, 304 PB |
| 81946 E12 | | | no Refseq gene within next 100 kb | |
| 77048 G07 | | EVI1 | | |
| 76771 H02 | | EVI1 | | |
| 77110 H11 | | EVI1 | | 241 PB |
| 77110 D02 | | EVI1 | | |
| 75916 D12 | | EVI1 | | 269 PB |
| 77048 E02 | | EVI1 | | |
| 76776 C04 | | EVI1 | | |
| 75917 C09 | | EVI1 | | |
| 75916 F04 | | EVI1 | | 157 CD15, 192 BM |
| 81520 F05 | | EVI1 | | 542 CD14 and CD15 |
| 75918 G04 | | EVI1 | | |
| 79207 B11 | | EVI1 | | |
| 76776 G04 | | MDS1 | | 381 PB |
| 81520 F05 | | MDS1 | | |
| 77049 G11 | | MDS1 | | 241 BM, 381 PB |
| 76776 E04 | | MDS1 | | |
| 89252 E08 | | MDS1 | | |
| 74718 H10 | | MDS1 | | 241 PB |
| 76776 A10 | | MDS1 | | 241 BM |
| 77509 A03 | | MDS1 | | 241 BM, 269 PB, 304 PB |
| 76062 D09 | | MDS1 | | |
| 74718 A07 | | MDS1 | | |
| 76062 E05 | | MDS1 | | |
| 75916 A01 | | MDS1 | | 241 PB and BM, 269 PB, 304 PB, |
| | | | | 339 PB, 381 PB, 416 PB, 542 |
| | | | | CD14 and CD15 and CD3 |
| 75917 B04 | | MDS1 | | 241 PB |
| 74718 G05 | | MDS1 | | |
| 76771 D05 | | MDS1 | | 269 PB |
| 77110 A09 | | MDS1 | | 192 BM, 241 PB, 269 PB, 339 |
| | | | | PB, 381 PB and CD15, 416 PB |
| 77049 B02 | | MDS1 | | 269 PB |
| 76776 A11 | | MDS1 | | 241 BM |
| 75385 B05 | | MDS1 | | |
| 78016 F03 | | MDS1 | | 416 PB |
| 78016 C11 | | MDS1 | | |
| 75917 H11 | | MDS1 | | 241 PB |
| 75916 A05 | | MDS1 | | 192 BFU-E6, 241 PB and BM, 304 |
| | | | | PB, 339 PB, 381 PB and CD15, |
| | | | | 416 PB, 472 PB |
| 78372 E08 | | MDS1 | | |
| 77110 F02 | | MDS1 | | |
| 77109 E01 | | MDS1 | | |
| 76776 G11 | | MDS1 | | 542 CD14 and CD15 |
| 77048 C07 | | MDS1 | | 192 CFU-GM1, 241 BM, 269 PB, |
| | | | | 339 PB, 416 PB |
| 75523 E11 | | MDS1 | | |
| 79208 F04 | | MDS1 | | |
| 76777 H12 | | BCL6 | 42029 bp upstream of MGC78665 | |
| | | | and 74268 bp upstream of SST | |
| 76776 B08 | | | no Refseq gene within next 100 kb | |
| 76771 D04 | | MIST | | |
| 76777 G01 | | | no Refseq gene within next 100 kb | |
| 76062 G02 | | STIM2 | | |
| 74718 D12 | | | no Refseq gene within next 100 kb | |
| 77051 G08 | | TEC | 78667 bp upstream of TXK | |
| 76778 D08 | | OCIAD1 | 70121 bp downstream of OCIAD2 | |
| 75523 C09 | | | no Refseq gene within next 100 kb | |
| 77051 C12 | | FLJ10808 | 38843 bp downstream of GNRHR and | |
| | | | 94744 bp downstream of BRDG1 | |
| 75916 F09 | | SEPT11 | | |
| 76774 A10 | | | no Refseq gene within next 100 kb | |
| 76776 A08 | | PRKG2 | | |
| 75921 E12 | | MLLT2 | | |
| 90189 A04 | | PGDS | 50785 bp downstream of SMARCAD1 | |
| 74718 H01 | | PGDS | | |
| 74718 H12 | | MANBA | | |
| 77048 C09 | 48214 | SRY1 | | 304 PB |
| 81507 F08 | | SET7 | 81594 bp downstream of RAB33B | |
| 74718 A09 | | MAML3 | | |
| 79274 D09 | 62465 | DCTD | | |
| 75921 H08 | | CARF | 81645 bp downstream of BOMB | |
| 76776 C06 | | IRF2 | | |
| 76777 C06 | | AHRR | 44356 bp upstream of SEC6L1 and | |
| | | | 74406 bp downstream of SLC9A3 and | |
| | | | 83930 bp downstream of PDCD6 | |
| 76771 A02 | 30971 | POLS | | |
| 76778 A06 | 20550 | ROPN1L | 50196 bp downstream of TEB4 | |
| 75916 H11 | | | no Refseq gene within next 100 kb | 192 PB |
| 81520 E07 | | | no Refseq gene within next 100 kb | |
| 76777 A03 | | FLJ10246 | 71881 bp upstream of MGC42105 | |
| | | | and 74954 bp downstream of | |
| | | | LOC153684 | |
| 75523 D07 | | IPO11 | 398 bp downstream of SLRN | |
| 75917 A04 | | C2GNT3 | | 101 G |
| 75921 H03 | | SSBP2 | 28722 bp upstream of CACH1 | |
| 75919 C10 | | EDIL3 | | |
| 76776 G10 | | SIAT8D | | |
| 81507 F03 | | FLJ20125 | 10422 bp upstream of KIAA0433 and | |
| | | | 89419 bp downstream of PAM | |
| 90189 A11 | 64124 | PTTG1 | 73775 bp upstream of SLU7 and | |
| | | | 92809 bp upstream of LOC63920 | |
| 75921 E11 | | | no Refseq gene within next 100 kb | |
| 90187 F03 | | STK10 | | |
| 76774 H08 | | GNB2L1 | 10147 bp downstream of TRIM41 and | |
| | | | 10433 bp downstream of TRIM52 and | |
| | | | 40778 bp upstream of TRIM7 and | |
| | | | 78333 bp upstream of FLJ45445 | |
| 75523 D12 | | C6orf105 | | |
| 75921 A12 | | | no Refseq gene within next 100 kb | |
| 74718 A11 | | JARID2 | | |
| 75921 D06 | | JARID2 | | |
| 76062 F03 | | C6orf32 | | |
| 90187 B07 | | BTN3A2 | 18685 bp upstream of BTN2A2 and | |
| | | | 37817 bp upstream of BTN3A1 and | |
| | | | 57678 bp upstream of BTN2A3 and | |
| | | | 76094 bp upstream of BTN3A3 | |
| 75921 E07 | | HCG9 | 20399 bp downstream of HLA-A | |
| 75523 G04 | | MGC57858 | 13109 bp downstream of NUDT3 and | |
| | | | 28882 bp downstream of HMGA1 | |
| 75916 F01 | | MGC57858 | 2866 bp downstream of NUDT3 and | |
| | | | 39125 bp downstream of HMGA1 | |
| 75917 C02 | | MGC57858 | 2470 bp downstream of NUDT3 and | |
| | | | 39521 bp downstream of HMGA1 | |
| 77512 B06 | | TBCC | 40114 bp upstream of KIAA0240 and | |
| | | | 58371 bp upstream of RDS and | |
| | | | 89769 bp downstream of C6orf133 | |
| | | | and 98991 bp upstream of RPL7L1 | |
| 89252 B10 | | RUNX2 | 118618 bp upstream of SUPT3H | |
| 75921 A09 | | SENP6 | | |
| 75916 B01 | | BACH2 | | |
| 76062 B08 | | BACH2 | | |
| 75921 F07 | | | no Refseq gene within next 100 kb | |
| 87515 E03 | | VNN3 | | |
| 77049 A09 | | REPS1 | | |
| 75921 E04 | | | no Refseq gene within next 100 kb | |
| 77049 G04 | | | no Refseq gene within next 100 kb | |
| 77110 H08 | | | no Refseq gene within next 100 kb | |
| 78017 D02 | | HDAC9 | | |
| 77509 D12 | | OSBPL3 | | |
| 76777 H11 | | HOXA7 | 11262 bp upstream of HOXA6 and | |
| | | | 3428 bp downstream of HOXA9 and | |
| | | | 11581 bp downstream of HOXA10 | |
| | | | and 15343 bp upstream of HOXA5 | |
| 78372 G08 | | CALN1 | | |
| 77509 D04 | | FKBP6 | 34725 bp upstream of MGC 45477 | |
| | | | and 34997 bp upstream of TRIM50C | |
| | | | and 49735 bp upstream of | |
| | | | WBSCR20C and 56157 bp | |
| | | | downstream of POM121 | |
| 76778 A01 | | NCF1 | 21901 bp downstream of GTF2IRD2B | |
| | | | and 97887 bp upstream of WBSCR16 | |
| 81520 F03 | | HIP1 | 37875 bp upstream of PMS2L3 | |
| 81518 G07 | 26476 | GNAI7 | | |
| 80484 C07 | | ZKSCAN1 | 22761 bp upstream of AZGP1 and | |
| | | | 50969 bp upstream of ZNF38 and | |
| | | | 65022 bp downstream of ZNF3 and | |
| | | | 90135 bp upstream of COPS6 | |
| 78372 E05 | | MGC50844 | 38301 bp upstream of SMO and | |
| | | | 74452 bp upstream of KIAA0828 and | |
| | | | 95214 bp upstream of TNPO3 | |
| 81518 E10 | | | no Refseq gene within next 100 kb | |
| 76778 B05 | | LOC346673 | 43891 bp upstream of HSPC049 and | |
| | | | 84715 bp upstream of MGC5242 and | |
| | | | 89512 bp downstream of FLJ110000 | |
| 76774 D11 | | CHRM2 | | |
| 76777 C10 | | ZH3HAV1 | 37787 bp upstream of FLJ12571 and | |
| | | | 59962 bp upstream of MGC14289 | |
| 77051 F04 | | REPIN1 | 9646 bp up of MGC33584 and | |
| | | | 28098 bp upstream of RARRES2 and | |
| | | | 31566 bp downstream of MGC3036 | |
| | | | and 81320 bp upstream of HIAN6 | |
| 76778 A07 | | SMARCD3 | | |
| 76778 E03 | | CTSB | | |
| 77509 C12 | 11127 | ADAM28 | 18535 bp upstream of ADAMDEC1 | |
| | | | and 75103 bp upstream of ADAM7 | |
| 88516 C02 | | PTK2B | | |
| 76778 B06 | | LYN | 39934 bp downstream of NCOA6IP | |
| 75921 G07 | | YTHDF3 | 41304 bp downstream of SPN | |
| 77051 E04 | | | no Refseq gene within next 100 kb | |
| 90271 F07 | | | no Refseq gene within next 100 kb | |
| 76062 H07 | | SMARCA2 | | |
| 76774 H04 | | C9orf93 | | |
| 77051 B10 | | NPR2 | 17138 bp downstream of SPAG8 and | |
| | | | 22313 bp upstream of HINT2 and | |
| | | | 38624 bp upstream of C9orf127 and | |
| | | | 41419 bp upstream of GBA2 | |
| 78372 H02 | | BTEB1 | 68041 bp downstream of SMC5L1 | |
| 75916 D02 | | ALDH1A1 | | |
| 74718 F02 | | ALDH1A1 | | |
| 76778 A02 | 64087 | AUH | | |
| 90189 F04 | | C9orf89 | 12281 bp downstream of SUSD3 and | |
| | | | 24085 bp downstream of NINJ1 and | |
| | | | 61181 bp downstream of FGD3 and | |
| | | | 87517 bp upstream of WNK2 | |
| 75916 E11 | | C9orf3 | 36111 bp downstream of FANCC | |
| 81518 A01 | | WDR31 | 8823 bp upstream of BSPRY and | |
| | | | 27976 bp downstream of HDHD3 and | |
| | | | 40871 bp downstream of ALAD and | |
| | | | 46402 bp upstream of MGC4734 | |
| 75523 E04 | | | no Refseq gene within next 100 kb | |
| 80484 H02 | | CEP1 | | |
| 76156 E08 | | GSN | 14870 bp upstream of GSN | |
| 76777 E09 | | RABGAP1 | 45497 bp upstream of GPR21 and | |
| | | | 57570 bp upstream of ZBTB26 and | |
| | | | 75740 bp upstream of ZNF482 | |
| 75917 A09 | | ZNF79 | 16103 bp downstream of SLC2A8 and | |
| | | | 27753 bp upstream of LRSAM1 and | |
| | | | 23691 bp downstream of RPL12 and | |
| | | | 30438 bp downstream of GARNL3 | |
| 76774 F06 | | | 6887 bp upstream of FLJ45983 | |
| 76777 F10 | 10542 | CUGBP2 | | |
| 76777 B06 | 16393 | C10orf7 | 70837 bp upstream of NUDT5 and | |
| | | | 82606 bp upstream of CAMK1D | |
| 75385 H07 | 82916 | PTPLA | | |
| 76774 E02 | | PLXD2 | | |
| 76062 F09 | | ACBD5 | | |
| 75918 C04 | | PRKG1 | | |
| 75523 G11 | | | no Refseq gene within next 100 kb | |
| 89252 G10 | | UNC5B | | |
| 90273 B07 | | CBARA1 | 54880 bp upstream of C10orf42 | |
| 76062 D04 | | MYST4 | | |
| 75916 C06 | | | no Refseq gene within next 100 kb | 157 PB |
| 76771 F05 | | IFIT1 | 22602 bp upstream of IFIT5 and | |
| | | | 51529 bp upstream of IFIT3 and | |
| | | | 38936 bp downstream of LOC387700 | |
| 76777 F11 | | C10orf129 | 23287 bp downstream of PDLIM1 and | |
| | | | 97486 bp downstream of SORBS1 | |
| 75921 D05 | | CUEDC2 | 13870 bp upstream of PSD and | |
| | | | 31072 bp downstream of NFKB2 and | |
| | | | 16255 bp downstream of C10orf95 | |
| | | | and 27831 bp upstream of C10orf77 | |
| | | | and 45648 bp downstream of | |
| | | | ACTR1A | |
| 75916 | | | no Refseq gene within next 100 kb | |
| B04a | | | | |
| 90189 D09 | 91938 | KIAA1598 | | |
| 77051 A08 | | FAM45A | 32330 bp in Intron8 of FAM45B and | |
| | | | 4478 bp downstream of SFXN4 and | |
| | | | 31266 bp downstream of PRDX3 and | |
| | | | 55648 bp upstream of EIF3S10 | |
| 75385 A05 | | GRK5 | 26911 bp upstream of PRDX3 and | |
| | | | 40072 bp upstream of SFXN4 | |
| 74718 F08 | | OR52NI | 68814 bp upstream of OR11-62 | |
| 76774 C09 | 24163 | OR2AG2 | 41194 bp upstream of OR2AG1 and | |
| | | | 50681 bp downstream of OR6A2 and | |
| | | | 60501 bp upstream of MRPL17 and | |
| | | | 88220 bp upstream of DCHS1 | |
| 76777 F08 | | ZNF143 | 13757 bp downstream of IPO7 | |
| 77051 D10 | | | no Refseq gene within next 100 kb | |
| 75919 E12 | | C11orf8 | | |
| 81946 A01 | | LMO2 | 97234 bp upstream of FBXO3 | |
| 75917 D11 | | LMO2 | | |
| 82772 A12 | | | no Refseq gene within next 100 kb | |
| 81519 H05 | 5907 | NGL-1 | | |
| 79207 C02 | | DGKZ | | |
| 75918 H03 | | NR1H3 | 4768 bp upstream of MADD | |
| 75921 F01 | | KBTBD4 | 2466 bp upstream of NDUFS3 and | |
| | | | 10830 bp downstream of C1QTNF4 | |
| 75916 C10 | 1783 | C1QTNF4 | 3320 bp downstream of NDUFS3 and | |
| | | | 8908 bp upstream of KBTBD4 | |
| 76778 C02 | | MGC5395 | 20202 bp downstream of SCGB1A1 | 157 CD15 |
| | | | and 50828 bp downstream of | |
| | | | ASRGL1 | |
| 75917 A03 | | ARRB1 | 19007 bp upstream of RPS3 | |
| 75921 H06 | | FLJ23441 | | |
| 86978 A03 | | GRM5 | | |
| 76776 G08 | 6125 | FGIF | 11858 bp upstream of MRE11A and | |
| | | | 38259 bp upstream of FUT4 and | |
| | | | 61666 bp upstream of PIWIL4 and | |
| | | | 104450 bp upstream of GPR83 | |
| 75919 B10 | | MAML2 | | |
| 81517 H07 | | | no Refseq gene within next 100 kb | |
| 76062 G05 | | | no Refseq gene within next 100 kb | |
| 76778 C01 | | LOC196264 | 1441 bp downstream of EVA1 and | 241 PB, 192 PB |
| | | | 38620 bp upstream of AMICA and | |
| | | | 75451 bp upstream of SCN2B and | |
| | | | 52758 bp upstream of CD3E | |
| 81947 E06 | | ETS1 | | |
| 90189 C08 | | FLI1 | | |
| 75523 G02 | | HSPC063 | | |
| 90188 H01 | | NINJ2 | 71141 bp downstream of BUGalNac- | |
| | | | T3 | |
| 74718 E01 | | ELKS | | |
| 75385 H01 | | CACNA2D4 | | |
| 75916 C01 | | CHD4 | 6165 bp downstream of GPR92 | |
| 74718 C04 | | EPS8 | | |
| 82771 C10 | | BCAT1 | 102251 bp upstream of LRMP | |
| 75916 H01 | | LRMP | | |
| 76776 F03 | | NEUROD4 | | |
| 74718 E10 | | NEUROD4 | | |
| 74718 C08 | | NEUROD4 | | |
| 75917 D08 | | RNF41 | 1880 bp upstream of MGC2731 | 122 PB |
| 81519 A11 | | FAM19A2 | | |
| 80484 E03 | | | no Refseq gene within next 100 kb | |
| 75917 C04 | | RASSF3 | | 381 PB |
| 75921 H01 | | | no Refseq gene within next 100 kb | |
| 81519 E07 | | PAMC1 | | |
| 75385 E07 | | PLXNC1 | | |
| 79274 B02 | | DAP13 | 17468 bp downstream of NR2C1 and | |
| | | | 76614 bp downstream of FGD6 | |
| 88516 A04 | | LTA4H | 91295 bp upstream of ELK3 and | |
| | | | 175128 bp downstream of PCTK2 | |
| 74718 C01 | | GNPTAB | 41160 bp upstream of SYCP3 and | |
| | | | 51561 bp downstream of CHPT1 and | |
| | | | 94749 bp downstream of MYBPC1 | |
| 90189 A03 | | GNPTAB | 41195 bp upstream of SYCP3 and | |
| | | | 51596 bp downstream of CHPT1 and | |
| | | | 94784 bp downstream of MYBPC1 | |
| 75921 D12 | | FLJ40142 | 40655 bp downstream of ANKRD13 | |
| | | | and 44294 bp upstream of CDV-1 and | |
| | | | 83788 bp upstream of GIT2 | |
| 75523 A11 | | JIK | 63714 bp upstream of SDS3 | |
| 76777 D01 | | CDK2AP1 | 14773 bp downstream of FLJ38663 | |
| | | | and 23176 bp downstream of SBNO1 | |
| | | | and 50838 bp upstream of | |
| | | | MPHOSPH9 | |
| 77048 H03 | | | no Refseq gene within next 100 kb | |
| 78017 G01 | 80625 | GPR133 | | |
| 90189 A06 | | CDADC1 | 16572 bp downstream of CAB39L | |
| 74718 G08 | | PCDH9 | | |
| 76776 F07 | | | no Refseq gene within next 100 kb | |
| 81520 D02 | | C13orf25 | | |
| 82772 A09 | | PHGDHL1 | 49822 bp upstream of EBI2 and | |
| | | | 98888 bp upstream of GPR18 | |
| 76777 F05 | | | no Refseq gene within next 100 kb | |
| 76062 D06 | | ABHD4 | 7354 bp upstream of DAD1 | |
| 75917 A01 | | AP4S1 | 910 bp upstream of STRN3 | |
| 81947 A05 | | EGLN3 | | |
| 82773 F09 | | PSMA6 | | |
| 75523 E08 | | MIA2 | | |
| 76776 B02 | | RPS29 | 10646 bp upstream of PPIL5 | |
| 81507 C04 | | GNG2 | 96271 bp downstream of FRMD6 | |
| 76776 C01 | | PSMC6 | 11170 bp upstream of ERO1L and | |
| | | | 23343 bp upstream of STYX and | |
| | | | 68323 bp downstream of GNPNAT1 | |
| 90271 H06 | | KIAA0586 | 72 bp in exon1 of TIMM9 and | |
| | | | 18741 bp downstream of UNQ9438 | |
| | | | and 54522 bp downstream of ARID4A | |
| 75916 D11 | 28274 | RTN1 | 62991 bp downstream of C14orf100 | |
| | | | and 83347 bp upstream of C14orf149 | |
| 76777 A08 | | MED6 | 69309 bp downstream of MAP3K9 | |
| 76777 C02 | | C14orf43 | 56848 bp upstream of PNMA1 and | |
| | | | 80698 bp upstream of ZADH1 and | |
| | | | 74784 bp downstream of C14orf168 | |
| 76778 D01 | | C14orf118 | 67790 bp downstream of MGC16028 | 192 PB and BM, 122 PB, 241 PB, |
| | | | | 269 PB, 381 PB |
| 81518 A06 | | RPS6KA5 | 50681 bp upstream of C14orf159 | |
| 74718 C05 | | | no Refseq gene within next 100 kb | |
| 75921 B12 | | ARHGAP11A | 60347 bp upstream of SGNE1 and | |
| | | | 88501 bp upstream of C15orf45 | |
| 80484 F05 | | PAK6 | 13763 bp downstream of BUB1B and | |
| | | | 53026 bp downstream of PLCB2 | |
| 75523 D06 | 6045 | B2M | 12320 bp upstream of RNF36 | |
| 76062 F07 | | ATP8B4 | | |
| 90189 C12 | | DAPK2 | 64373 bp downstream of FLJ22875 | |
| | | | and 87787 bp upstream of SNX1 | |
| 76062 H02 | 315 | TRIP4 | 74186 bp upstream of KIAA0101 | |
| 76777 A12 | 48282 | TRIP4 | | |
| 75385 F08 | | CSK | | |
| 76778 H02 | | CSK | | 122 PB |
| 81518 A05 | 35684 | MESDC1 | 59720 bp upstream of C15orf26 and | |
| | | | 88030 bp downstream of KIAA1199 | |
| 76062 F04 | 24932 | RKHD3 | | |
| 79274 B07 | | AKAP13 | | |
| 77051 G04 | | DET1 | 68794 bp downstream of MRPS11 | |
| | | | and 74305 bp upstream of FLJ12484 | |
| | | | and 79665 bp upstream of MRPL46 | |
| 75917 B10 | | DKFZp547K1113 | 37415 bp downstream of IDH2 | |
| 76776 A09 | 73841 | SV2B | | |
| 76771 A01 | 51315 | LRRK1 | 54307 bp downstream of CHSY1 | |
| 75917 B03 | 39664 | CHSY1 | 65958 bp downstream of LRRK1 | 381 PB |
| 76777 G02 | | TRAF7 | 1016 bp downstream of RAB26 and | |
| | | | 19257 bp upstream of PKD1 and | |
| | | | 22028 bp downstream of CASKIN1 | |
| | | | and 50339 bp upstream of GBL | |
| 76778 B03 | | ZNF205 | 22026 bp upstream of ZNF213 and | |
| | | | 20252 bp upstream of ZNF206 and | |
| | | | 43565 bp downstream of NK4 and | |
| | | | 52391 bp downstream of MMP25 | |
| 76771 B12 | | ABCC1 | 73087 bp downstream of ABCC6 | |
| 74718 C07 | | THUMPD1 | | |
| 79207 C11 | | IL21R | 36451 bp downstream of IL4R and | |
| | | | 59523 bp downstream of GTF3C1 | |
| 76777 B03 | | | no Refseq gene within next 100 kb | |
| 76778 B02 | | MGC2474 | 8555 bp upstream of FLJ23436 and | |
| | | | 11960 bp downstream of ITGAL and | |
| | | | 18621 bp downstream of MGC13138 | |
| | | | and 89241 bp upstream of SEPHS2 | |
| 81520 H08 | | N4BP1 | | |
| 81507 A12 | | SLIC1 | 13404 bp upstream of Card 15 and | |
| | | | 58339 bp upstream of CYLD and | |
| | | | 49005 bp downstream of NKD1 | |
| 81520 C11 | | CDH9 | | |
| 76062 D03 | | GPR56 | | |
| 76774 A02 | | GPR56 | 25316 bp upstream of GPR97 and | |
| | | | 51871 bp upstream of DKFZp434I099 | |
| | | | and 94262 bp upstream of KATNB1 | |
| | | | and 65757 bp downstream of | |
| | | | GPR114 | |
| 76774 G12 | | ATBF1 | | 339 PB |
| 78017 G07 | | | no Refseq gene within next 100 kb | |
| 77051 G06 | | ZNRF1 | 90959 bp downstream of LDHD | |
| 81947 C08 | | MAF | | |
| 76776 H12 | | CMIP | | |
| 75385 B02 | 46711 | MGC22001 | | 65 PB |
| 77048 G02 | | C17orf31 | 38695 bp downstream of FLJ14069 | 241 BM, 381 PB |
| | | | and 88280 bp upstream of SRR | |
| 81507 C01 | 22630 | OR1A1 | 38793 bp downstream of OR3A2 and | |
| | | | 40730 bp downstream of OR1A2 and | |
| | | | 52458 bp downstream of OR3A1 and | |
| | | | 71133 bp upstream of OR3A4 | |
| 78017 B03 | | SAT2 | 1983 bp upstream of SHBG and | |
| | | | 13437 bp upstream of FXR2 and | |
| | | | 22746 bp upstream of ATP1B2 and | |
| | | | 38021 bp upstream of SOX15 | |
| 89253 D10 | | ADORA2B | 55081 bp upstream of TTC19 | |
| 74718 F12 | | ADORA2B | | 21 PB |
| 75523 H11 | | TRPV2 | 41597 bp upstream of MGC40157 | 80 PB |
| | | | and 14711 bp downstream of UBB | |
| 75916 D01 | | NF1 | 6461 bp in Intron1 of EVI2B and | |
| | | | 7422 bp downstream of EVI2A and | |
| | | | 12716 bp upstream of OMG | |
| 74718 B07 | | LOC117584 | | |
| 76777 D08 | | MLLT6 | | 157 CD15 |
| 75921 E01 | | STAT5A | 1374 bp upstream of STAT5B | |
| 74718 B04 | | STAT3 | | |
| 75385 E05 | | HOXB3 | | |
| 90273 H04 | 35840 | STXBP4 | 65087 bp upstream of HLF | |
| 75523 E10 | 3711 | HLF | 65377 bp downstream of MMD | |
| 76776 D10 | | MAP3K3 | 19933 bp downstream of MGC10986b | |
| | | | and 26867 bp downstream of LYK5 | |
| 76774 A06 | | SCN4A | 18670 bp downstream of ICAM2 and | |
| | | | 51572 bp upstream of CD79B and | |
| | | | 65088 bp upstream of GH1 and | |
| | | | 59474 bp downstream of ERN1 | |
| 75921 F02 | | ABCA10 | 18184 bp downstream of ABCA5 | |
| 80484 A06 | | EPB41L3 | | 269 PB |
| 76778 C09 | | | no Refseq gene within next 100 kb | |
| 76062 E09 | | | no Refseq gene within next 100 kb | |
| 90188 B01 | | ZNF521 | | |
| 76776 D08 | | ZNF521 | | |
| 76774 B12 | | RNF125 | 50567 bp upstream of RNF138 and | |
| | | | 98201 bp upstream of KIAA1012 | |
| 75523 B10 | | | no Refseq gene within next 100 kb | |
| 76778 G07 | | SETBP1 | | 241 BM, 304 PB |
| 79274 B06 | | SETBP1 | | |
| 77512 B07 | | SETBP1 | | |
| 76778 F12 | | SETBP1 | | 269 PB, 304 PB |
| 76776 E09 | | SETBP1 | | |
| 75916 G10 | | SETBP1 | | 241 PB |
| 77509 D02 | | SETBP1 | | |
| 76778 E06 | 34267 | FLJ20071 | 58824 bp upstream of SMAD7 | |
| 75921 F04 | | CDH7 | | |
| 75917 F11 | | | no Refseq gene within next 100 kb | |
| 77109 G08 | | MBP | 91584 bp downstream of ZNF236 | |
| 77109 C08 | | MBP | 91634 bp downstream of ZNF236 | |
| 76777 G11 | | NFATC1 | | |
| 77051 C08 | | GAMT | 4573 bp upstream of DAZAP1 and | |
| | | | 7429 bp downstream of NDUFS7 and | |
| | | | 35368 bp upstream of RPS15 and | |
| | | | 24584 bp downstream of MUM1 | |
| 75921 E06 | | MOBKL2A | | |
| 77051 A04 | | NFIC | 44590 bp downstream of BRUNOL5 | |
| 86978 G01 | | RAB3D | 3292 bp downstream of TSPAN16 | |
| 76774 C11 | | CALR | 3311 bp upstream of FARSLA and | |
| | | | 8821 bp upstream of RAD23A and | |
| | | | 17139 bp downstream of | |
| | | | GADD45GIP1 and 32599 bp | |
| | | | upstream of FLJ38607 | |
| 77051 C01 | | GPSN2 | 10723 bp upstream of DNAJB1 and | |
| | | | 32980 bp upstream of RGS19IP1 and | |
| | | | 36967 bp downstream of NDUFB7 | |
| | | | and 56645 bp upstream of PTGER1 | |
| 76776 E10 | | ZNF382 | 214002 bp in Intron4 of MGC62100 | 122 PB, 241 PB |
| | | | and 13004 bp downstream of G10T-1 | |
| 78372 H05 | 14238 | EGLN2 | 20870 bp downstream of CYP2A6 | |
| | | | and 52770 bp downstream of | |
| | | | CYP2A7 and 45178 bp downstream | |
| | | | of MIA and 57282 bp downstream of | |
| | | | SNRPA | |
| 81519 H11 | 13 | AKT1S1 | 1466 bp upstream of PNKP and | |
| | | | 8287 bp downstream of PTOV1 and | |
| | | | 19716 bp upstream of TBC1D17 and | |
| | | | 21542 bp downstream of IL4I1 | |
| 81507 F06 | | LAIR1 | 28550 bp upstream of TTYH1 and | |
| | | | 47664 bp upstream of ILT7 and | |
| | | | 62315 upstream of LENG8 and | |
| | | | 73717 bp upstream of LIR9 | |
| 76777 G09 | 30168 | ZNF579 | 44015 bp downstream of FLJ14768 | |
| | | | and 52999 bp upstream of ZNF524 | |
| | | | and 73376 bp downstream of | |
| | | | LOC147808 and 59796 bp upstream | |
| | | | of KLP1 | |
| 78372 C09 | | SOX12 | | |
| 76156 C09 | 20773 | C20orf30 | 35871 bp downstream of PCNA and | |
| | | | 47754 bp upstream of CDS2 and | |
| | | | 77583 bp upstream of SLC23A2 | |
| 75917 H01 | | PLCB1 | | |
| 77051 A03 | | SNX5 | 8324 bp upstream of C20orf72 and | |
| | | | 63358 bp downstream of ZNF339 | |
| 78017 G05 | 29747 | LOC200261 | 75457 bp downstream of C1QR1 | |
| 77051 A11 | | ZNF336 | 6263 bp downstream of NXT1 and | |
| | | | 13495 bp downstream of NAPB and | |
| | | | 78651 bp upstream of CSTL1 and | |
| | | | 89370 bp downstream of CST11 | |
| 75921 | 6871 | BAK1 | 20678 bp downstream of COMMD7 | |
| 78017 C11 | 12276 | SPAG4L | 36101 bp upstream of BPIL1 and | |
| | | | 60148 bp upstream of BPIL3 and | |
| | | | 83924 bp upstream of C20orf185 | |
| 78372 C06 | | EPB41IL1 | 33711 bp downstream of C20orf152 | |
| 76777 D02 | | NCOA3 | | |
| 75917 B01 | | KIAA1404 | | |
| 76062 G10 | 6093 | STIMN3 | 13739 bp upstream of GMEB2 and | |
| | | | 24679 bp upstream of C20orf41 | |
| 78017 D06 | | | no Refseq gene within next 100 kb | |
| 83397 G03 | | CYYR1 | | |
| 76774 B07 | 3437 | CBR3 | 14548 bp upstream of C21orf5 and | |
| | | | 76829 bp downstream of CBR1 and | |
| | | | 89608 bp upstream of C21orf18 | |
| 81507 A02 | | DYRK1A | 88330 bp upstream of DSCR3 | |
| 82771 E03 | | ERG | | |
| 76777 H06 | | CSTB | 12623 bp upstream of D21S2056E | |
| | | | and 28837 bp downstream of | |
| | | | LOC284837 and 38067 bp | |
| | | | downstream of C21orf124 and | |
| | | | 14628 bp downstream of PDXK | |
| 77509 F01 | | C22orf14 | 10822 bp upstream of SLC2A11 | |
| | | | 11536 bp downstream of SMARCB1 | |
| | | | 48328 bp upstream of MIF | |
| | | | 61735 bp downstream of MMP11 | |
| 76774 H01 | | UPB1 | 24922 bp downstream of C22orf13 | |
| | | | and 40130 bp upstream of SNRPD3 | |
| | | | and 68230 bp upstream of GGT1 and | |
| | | | 73164 bp downstream of ADORA2A | |
| 90187 A06 | 20676 | MN1 | | |
| 81507 D03 | 974 | MN1 | | |
| 76774 D10 | | MN1 | 66354 bp downstream of PITNB | |
| 75523 B09 | | XBP1 | | |
| 76774 B05 | | C22orf19 | 718 bp downstream of NIPSNAP1 | |
| | | | and 49483 bp upstream of NF2 and | |
| | | | 62807 bp downstream of NEFH | |
| 79208 A01 | | | 28118 bp downstream of FLJ38628 | |
| | | | and 46462 bp downstream of | |
| | | | MGC17330 and 90672 bp | |
| | | | downstream of ZNF278 and 94649 bp | |
| | | | upstream of PLA2G3 | |
| 77051 C02 | | RBM9 | | |
| 77512 E06 | | MYH9 | 62820 bp downstream of APOL1 and | |
| | | | 90397 bp upstream of APOL2 | |
| 76776 F10 | | TXN2 | 7110 bp downstream of FLJ23322 | |
| | | | and 29712 bp downstream of EIF3S7 | |
| | | | and 82918 bp downstream of | |
| | | | CACNG2 | |
| 75921 F12 | | PSCD4 | | |
| 77051 B02 | | UNC84B | 24592 bp downstream of DNAL4 and | |
| | | | 21678 bp downstream of GTPBP1 | |
| | | | and 53653 bp upstream of KIAA0063 | |
| | | | and 69433 bp downstream of | |
| | | | TOMM22 | |
| 90189 G10 | | MSL3L1 | | |
| 80484 D12 | | | no Refseq gene within next 100 kb | |
| 77109 C04 | 11680 | FLJ2544 | 43270 bp upstream of MGC4825 and | |
| | | | 103761 bp upstream of EIF2S3 | |
| 81517 F05 | 2277 | ZCCHC5 | | |
| 77048 H08 | | MST4 | | |
| 75916 B02 | | IDS | 24884 bp upstream of LOC91966 | 304 PB |
| 75523 B01 | | UTY | | |
| 76774 B06 | | | no Refseq gene within next 100 kb | |

TABLE 1b

| | Days | | | | | Se- | | | | | | | |
|---|---|---|---|---|---|---|---|---|---|---|---|---|---|
| | Post- | | Ge- | | | quence | | Upstream | In Gene, | Downstream | Next RefSeq | | Additionally |
| Sequence | trans- | | nomic | Identity | Chromo- | Orien- | Integration | of TSS | Distance to | of | Gene (within | | Detected at Days |
| Identity | plant | Sample | Length | [%] | some | tation | Locus | [bp] | TSS [bp] | Gene [bp] | 100 kb) | More RefSeq Genes within 100 kb | Posttransplant |
| 78169 D06 | 84 | PB | 55 | 100 | 1 | minus | 2279080 | | | 7462 | SKI | 5778 bp downstream of FLJ13941 | |
| | | | | | | | | | | | | and 76411 bp upstream of RER1 and | |
| | | | | | | | | | | | | 89325 bp downstream of PEX10 | |
| 78166 C09 | 149 | PB | 31 | 100 | 1 | minus | 3011985 | | 3084 In1 | | PRDM16 | | |
| 78166 B07 | 149 | PB | 331 | 98.6 | 1 | plus | 3109761 | | 100860 In1 | | PRDM16 | | 245 PB |
| 82774 D06 | 287 | PB | 48 | 100 | 1 | plus | 3109929 | | 101028 In1 | | PRDM16 | | |
| 78165 H02 | 149 | PB | 157 | 99.4 | 1 | minus | 3111506 | | 102605 In1 | | PRDM16 | | 175 PB, 245 PB, 343 PB |
| 78165 B07 | 149 | PB | 72 | 98.7 | 1 | plus | 3113799 | | 104898 In1 | | PRDM16 | | |
| 78373 B06 | 149 | PB | 46 | 100 | 1 | minus | 3121364 | | 112463 In1 | | PRDM16 | | |
| 81841 E09 | 245 | PB | 93 | 100 | 1 | minus | 3121907 | | 113006 In1 | | PRDM16 | | |
| 78373 G04 | 149 | PB | 27 | 100 | 1 | minus | 3123391 | | 114490 In1 | | PRDM16 | | |
| 79275 E09 | 175 | PB | 44 | 100 | 1 | plus | 3123459 | | 114558 In1 | | PRDM16 | | 245 PB |
| 78373 F04 | 149 | PB | 140 | 99.3 | 1 | plus | 3123555 | | 114654 In1 | | PRDM16 | | |
| 81673 C08 | 245 | PB | 135 | 100 | 1 | plus | 3123617 | | 114716 In1 | | PRDM16 | | 343 PB |
| 79275 B07 | 175 | PB | 91 | 100 | 1 | plus | 3123716 | | 114815 In1 | | PRDM16 | | |
| 79272 F07 | 175 | PB | 94 | 100 | 1 | minus | 3123809 | | 114908 In1 | | PRDM16 | | |
| 79275 D06 | 175 | PB | 273 | 99.7 | 1 | plus | 3123898 | | 114997 In1 | | PRDM16 | | |
| 78166 D04 | 149 | PB | 104 | 100 | 1 | plus | 3124033 | | 115132 In1 | | PRDM16 | | 175 PB, 245 PB, 287 PB |
| 78166 H04 | 149 | PB | 211 | 99.6 | 1 | plus | 3124270 | | 115369 In1 | | PRDM16 | | 287 PB |
| 78373 E04 | 149 | PB | 90 | 98.9 | 1 | plus | 3124373 | | 115472 In1 | | PRDM16 | | 175 PB, 245 PB |
| 78373 H05 | 149 | PB | 142 | 100 | 1 | plus | 3124425 | | 115524 In1 | | PRDM16 | | |
| 78168 E09 | 28 | BM | 111 | 100 | 1 | minus | 7955365 | 694 | | | PARK7 | 20212 bp upstream of TNFRSF9 and | |
| | | | | | | | | | | | | 50696 bp downstream of MIG6 | |
| 76857 G01 | 84 | BM | 129 | 100 | 1 | minus | 28655278 | | 2094 Ex2 | | RCC1 | 7843 bp downstream of PHACTR4 | |
| | | | | | | | | | | | | and 44943 bp upstream of SECP43 | |
| | | | | | | | | | | | | and 84050 bp upstream of | |
| | | | | | | | | | | | | MGC45806 and 94951 bp | |
| | | | | | | | | | | | | downstream of TAF12 | |
| 76857 E05 | 28 | BM | 46 | 100 | 1 | plus | 35231210 | | 17125 In4 | | ZMYM1 | 64826 bp upstream of ZNF258 and | |
| | | | | | | | | | | | | 87086 bp downstream of SFPQ | |
| 78168 A10 | 28 | BM | 145 | 100 | 1 | plus | 38057477 | | 24309 In6 | | INPP5B | 34270 bp downstream of SF3A3 and | |
| | | | | | | | | | | | | 63153 bp upstream of MTF1 and | |
| | | | | | | | | | | | | 74059 bp downstream of FHL3 and | |
| | | | | | | | | | | | | 90030 bp upstream of CGI-94 | |
| 86758 B11 | 343 | PB | 53 | 100 | 1 | plus | 53858392 | | 53506 In1 | | GLIS1 | 87018 bp downstream of TMEM48 | |
| 81676 B06 | 245 | PB | 89 | 100 | 1 | minus | 53861213 | | 50685 In1 | | GLIS1 | 84197 bp downstream of TMEM48 | 287 PB, 343 PB |
| 85439 G04 | 245 | CFU-GM5 | 196 | 99.5 | 1 | plus | 109109739 | | 2009 In1 | | C1orf62 | 22130 bp upstream of GPSM2 and | |
| | | | | | | | | | | | | 45549 bp downstream of STXBP and | |
| | | | | | | | | | | | | 76911 bp downstream of MCLC | |
| 78169 H10 | 84 | PB | 28 | 100 | 1 | plus | 150575138 | | | 9433 | SLC27A3 | 15506 bp downstream of FLJ21919 | |
| | | | | | | | | | | | | and 17040 bp upstream of P66BETE | |
| | | | | | | | | | | | | and 95602 bp downstream of NPR1 | |
| 78373 H04 | 149 | PB | 396 | 99.5 | 1 | minus | 152346689 | 1463 | | | ASH1L | 46391 bp upstream of FLJ10504 and | |
| | | | | | | | | | | | | 95630 bp downstream of YAP | |
| 78373 A09 | 149 | PB | 223 | 98.6 | 1 | plus | 154185096 | | | | | no Refseq gene within next 100 kb | |
| 81840 A09 | 245 | PB | 72 | 100 | 1 | minus | 157424179 | | 5763 In1 | | SLAMF1 | 37432 bp downstream of CD48 and | |
| | | | | | | | | | | | | 61843 bp upstream of CD84 and | |
| | | | | | | | | | | | | 97971 bp upstream of SLAMF7 | |
| 76856 E09 | 24 | PB | 64 | 100 | 1 | plus | 161305508 | | 44913 In2 | | PBX1 | | |
| 78166 E09 | 149 | PB | 36 | 95.9 | 1 | minus | 190816349 | | | | | no Refseq gene within next 100 kb | |
| 86758 F02 | 343 | PB | 49 | 100 | 1 | minus | 219722402 | | | 43735 | SUSD4 | | |
| 78169 H04 | 84 | PB | 168 | 100 | 1 | minus | 231294540 | | | | | no Refseq gene within next 100 kb | |
| 78168 E03 | 28 | PB | 79 | 100 | 1 | plus | 243192551 | 20868 | | | CGI-49 | 36084 bp downstream of FLJ32001 | |
| | | | | | | | | | | | | and 85886 bp upstream of | |
| | | | | | | | | | | | | LOC149134 | |
| 78168 B05 | 28 | PB | 85 | 85.8 | 1 | minus | 243323810 | | | 4904 | ELYS | 45373 bp downstream of LOC149134 | |
| | | | | | | | | | | | | and 66332 bp downstream of CGI-49 | |
| 76856 B06 | 24 | PB | 125 | 99.2 | 2 | minus | 7001447 | 6674 | | | RNF144 | 12932 bp downstream of CIG5 and | |
| | | | | | | | | | | | | 44811 bp upstream of LOC129607 | |
| 76856 A05 | 24 | PB | 146 | 100 | 2 | minus | 7002213 | 5908 | | | RNF144 | 13698 bp downstream of CIG5 and | 28 PB |
| | | | | | | | | | | | | 45577 bp upstream of LOC129607 | |
| 76857 H03 | 84 | BM | 37 | 100 | 2 | plus | 7991761 | | | | | no Refseq gene within next 100 kb | |
| 81673 H10 | 245 | PB | 159 | 99.4 | 2 | minus | 16579265 | | | 76264 | FAM49A | | |
| 78169 A11 | 84 | BM | 194 | 100 | 2 | plus | 29251151 | | 1182 In1 | | FLJ21069 | 76142 bp downstream of ALK and | |
| | | | | | | | | | | | | 69087 bp downstream of LOC165186 | |
| 78168 F10 | 28 | BM | 74 | 100 | 2 | plus | 38890240 | 138 | | | SFRS7 | | |
| 78165 F12 | 149 | PB | 79 | 100 | 2 | plus | 43100603 | | | | | no Refseq gene within next 100 kb | 245 PB, 287 PB |
| 78169 B05 | 84 | PB | 582 | 99.7 | 2 | minus | 54602518 | 62640 | | | SPTBN1 | 63840 bp upstream of DKFZp547I014 | |
| 82776 B02 | 287 | PB | 173 | 100 | 2 | plus | 62616576 | | | 22837 | TMEM17 | | |
| 76855 H03 | 24 | PB | 114 | 100 | 2 | plus | 68871995 | 1624 | | | ARHGAP25 | 77636 bp downstream of GPR73 | |
| 77510 A03 | 84 | PB | 65 | 100 | 2 | plus | 70261280 | | | 27390 | FLJ20558 | 89187 bp downstream of TIA1 and | |
| | | | | | | | | | | | | 33297 bp downstream of PCBP1 | |
| 81840 A12 | 245 | PB | 136 | 100 | 2 | minus | 85547501 | | 1631 In1 | | CAPG | 10211 bp upstream of LOC284948 | |
| | | | | | | | | | | | | and 16974 bp downstream of RBED1 | |
| | | | | | | | | | | | | and 66621 bp upstream of RetSat | |
| | | | | | | | | | | | | and 80469 bp upstream of TGOLN2 | |
| 78165 A10 | 149 | PB | 85 | 100 | 2 | plus | 130845426 | | 15578 In12 | | PTPN18 | 24999 bp downstream of IMP4 and | |
| | | | | | | | | | | | | 29274 bp upstream of MGC12981 | |
| 78373 C12 | 149 | PB | 37 | 100 | 2 | plus | 148215862 | | | | | no Refseq gene within next 100 kb | |
| 76856 C04 | 24 | PB | 25 | 100 | 2 | minus | 161059334 | | 116478 In1 | | RBMS1 | | 49 PB |
| 78168 G04 | 28 | PB | 95 | 100 | 2 | plus | 197985703 | 97435 | | | LOC91526 | 96503 bp downstream of SF3B1 | |
| 77511 A05 | 84 | BM | 79 | 100 | 2 | plus | 200406701 | | | | | no Refseq gene within next 100 kb | |
| 78169 G05 | 84 | PB | 70 | 98.6 | 2 | plus | 207841498 | | 14622 In1 | | KLF7 | | |
| 82776 C08 | 287 | PB | 258 | 100 | 2 | minus | 210858935 | | 2622 In1 | | FLJ23861 | 19289 bp downstream of ACADL | |
| 77510 E05 | 84 | PB | 114 | 100 | 2 | plus | 237247757 | 12686 | | | CMKOR1 | 49665 bp upstream of IQCA | |
| 76855 E03 | 24 | PB | 117 | 100 | 3 | minus | 3210317 | 13927 | | | CRBN | 42789 bp downstream of TRNT1 and | |
| | | | | | | | | | | | | 83286 bp upstream of IL5RA | |
| 82775 G01 | 287 | PB | 93 | 95.6 | 3 | minus | 4514260 | | 4124 In2 | | ITPR1 | | |
| 78165 C09 | 149 | PB | 39 | 100 | 3 | plus | 16411665 | | 118561 In4 | | RAFTLIN | 91553 bp downstream of MGC15763 | |
| 86611 G05 | 119 | PB | 163 | 100 | 3 | minus | 33114514 | 882 | | | GLB1 | 15969 bp upstream of CRTAP | |
| 78165 G10 | 149 | PB | 255 | 99.3 | 3 | plus | 45035362 | 7412 | | | TNA | 7396 bp downstream of EXOSC7 and | |
| | | | | | | | | | | | | 42744 bp upstream of ZDHHC3 and | |
| | | | | | | | | | | | | 63412 bp downstream of CDCP1 and | |
| | | | | | | | | | | | | 99446 bp upstream of FLJ20209 | |
| 77510 C04 | 84 | PB | 239 | 100 | 3 | minus | 46997971 | 4728 | | | CCDC12 | 34955 bp downstream of HYPB and | |
| | | | | | | | | | | | | 77680 bp downstream of PTHR1 | |
| 78373 F05 | 149 | PB | 115 | 99.2 | 3 | minus | 61212582 | 418 | | | FHIT | | |
| 81675 D12 | 245 | PB | 27 | 96.3 | 3 | minus | 87222527 | 99580 | | | VGL-3 | | 287 PB |
| 89684 D02 | 245 | CFU-GM5 | 78 | 98.8 | 3 | minus | 103278639 | | | | | no Refseq gene within next 100 kb | |
| 89684 C12 | 245 | CFU-GM5 | 95 | 98.2 | 3 | plus | 103279039 | | | | | no Refseq gene within next 100 kb | |
| 88283 C01 | 343 | PB | 223 | 100 | 3 | plus | 109327673 | | | 34677 | ESRRBL1 | 35048 bp upstream of CD47 | |
| 77510 F08 | 84 | PB | 63 | 100 | 3 | plus | 162172682 | | 129979 In3 | | PPM1L | | |
| 81674 A11 | 245 | PB | 40 | 100 | 3 | minus | 170336451 | | 10344 In2 | | EVI1 | | |
| 86758 H12 | 343 | PB | 89 | 100 | 3 | minus | 170337216 | | 9579 In2 | | EVI1 | | |
| 82776 B11 | 287 | PB | 252 | 98.6 | 3 | minus | 170338758 | | 8037 In2 | | EVI1 | | |
| 78165 E09 | 149 | PB | 197 | 99.5 | 3 | minus | 170338858 | | 7937 In2 | | EVI1 | | 287 PB |
| 78166 B03 | 149 | PB | 158 | 99.4 | 3 | minus | 170339841 | | 6954 In2 | | EVI1 | | 175 PB |
| 79275 G07 | 175 | PB | 83 | 100 | 3 | minus | 170342651 | | 4144 In2 | | EVI1 | | |
| 78166 H11 | 149 | PB | 123 | 100 | 3 | minus | 170345961 | | 834 In1 | | EVI1 | | |
| 81673 A07 | 245 | PB | 75 | 100 | 3 | minus | 170347808 | 1013 | | | EVI1 | | |
| 86611 G04 | 119 | PB | 300 | 99.4 | 3 | plus | 170348090 | 1295 | | | EVI1 | | |
| 88283 H11 | 343 | PB | 87 | 100 | 3 | minus | 170348423 | | | 1165 | MDS1 | | |
| 85439 A02 | 245 | CFU-GM1 | 219 | 100 | 3 | plus | 170350896 | | 513280 In2 | | MDS1 | | |
| 81673 H07 | 245 | PB | 65 | 100 | 3 | plus | 170352049 | | 512127 In2 | | MDS1 | | |
| 87429 F02 | 343 | PB | 204 | 100 | 3 | minus | 170355075 | | 509101 In2 | | MDS1 | | |
| 81673 D07 | 245 | PB | 150 | 100 | 3 | minus | 170366741 | | 497435 In2 | | MDS1 | | 287 PB |
| 81673 F06 | 245 | PB | 24 | 100 | 3 | plus | 170396907 | | 467269 In2 | | MDS1 | | |
| 81676 B02 | 245 | PB | 47 | 100 | 3 | minus | 170415074 | | 449102 In2 | | MDS1 | | 245 CFU-GM2 |
| 87429 A02 | 343 | PB | 118 | 100 | 3 | plus | 170415363 | | 448813 In2 | | MDS1 | | 245 CFU-GM6 |
| 81674 B09 | 245 | PB | 62 | 100 | 3 | plus | 170444820 | | 419356 In2 | | MDS1 | | |
| 82776 E03 | 287 | PB | 81 | 100 | 3 | plus | 170445204 | | 418972 In2 | | MDS1 | | 343 PB |
| 78166 E03 | 149 | PB | 178 | 98.4 | 3 | minus | 170449882 | | 414294 In2 | | MDS1 | | |
| 82774 G01 | 287 | PB | 19 | 100 | 3 | minus | 170450331 | | 413845 In2 | | MDS1 | | |
| 81674 A12 | 245 | PB | 176 | 100 | 3 | minus | 170508606 | | 355570 In2 | | MDS1 | | 245 CFU-GM1, |
| | | | | | | | | | | | | | 287 PB, 343 PB |
| 81674 D05 | 245 | PB | 41 | 100 | 3 | plus | 170534821 | | 329355 In2 | | MDS1 | | 287 PB, 343 PB |
| 81676 C08 | 245 | PB | 82 | 100 | 3 | minus | 170536132 | | 328044 In2 | | MDS1 | | |
| 78166 D08 | 149 | PB | 79 | 100 | 3 | plus | 170536218 | | 327958 In2 | | MDS1 | | |
| 81676 B08 | 245 | PB | 118 | 100 | 3 | minus | 170545797 | | 327537 In2 | | MDS1 | | |
| 81675 E02 | 245 | PB | 25 | 100 | 3 | plus | 170546186 | | 317990 In2 | | MDS1 | | 245 CFU-GM4, |
| | | | | | | | | | | | | | 287 PB, 343 PB |
| 81674 G07 | 245 | PB | 69 | 100 | 3 | minus | 170548107 | | 316069 In2 | | MDS1 | | 343 PB |
| 78165 D10 | 149 | PB | 70 | 98.6 | 3 | minus | 170552880 | | 311296 In2 | | MDS1 | | 245 PB, 287 PB, 343 PB |
| 81674 A02 | 245 | PB | 141 | 100 | 3 | plus | 170553197 | | 310979 In2 | | MDS1 | | |
| 82774 B05 | 287 | PB | 71 | 100 | 3 | plus | 170554755 | | 309421 In2 | | MDS1 | | |
| 86612 A01 | 287 | PB | 76 | 98.7 | 3 | minus | 170555336 | | 308840 In2 | | MDS1 | | |
| 78166 B04 | 149 | PB | 78 | 100 | 3 | minus | 170555455 | | 308721 In2 | | MDS1 | | 175 PB, 245 PB, |
| | | | | | | | | | | | | | 287 PB, 343 PB |
| 87429 A09 | 343 | PB | 78 | 100 | 3 | minus | 170555532 | | 308644 In2 | | MDS1 | | |
| 81674 A05 | 245 | PB | 95 | 100 | 3 | minus | 170555633 | | 308543 In2 | | MDS1 | | |
| 82774 G04 | 287 | PB | 70 | 100 | 3 | plus | 170556130 | | 308046 In2 | | MDS1 | | |
| 81674 G12 | 245 | PB | 22 | 100 | 3 | plus | 170556199 | | 307977 In2 | | MDS1 | | 287 PB |
| 86758 B06 | 343 | PB | 143 | 100 | 3 | plus | 170556399 | | 307777 In2 | | MDS1 | | |
| 78165 B06 | 149 | PB | 124 | 99.2 | 3 | plus | 170557382 | | 306794 In2 | | MDS1 | | |
| 81676 A05 | 245 | PB | 65 | 100 | 3 | plus | 170557818 | | 306358 In2 | | MDS1 | | 287 PB |
| 78166 G05 | 149 | PB | 53 | 100 | 3 | plus | 170559264 | | 304912 In2 | | MDS1 | | 287 PB |
| 82774 C01 | 287 | PB | 30 | 100 | 3 | minus | 170562559 | | 301617 In2 | | MDS1 | | 343 PB |
| 81676 A06 | 245 | PB | 110 | 100 | 3 | plus | 170588247 | | 275929 In1 | | MDS1 | | |
| 79275 E08 | 175 | PB | 35 | 100 | 3 | plus | 170588540 | | 275636 In1 | | MDS1 | | 245 PB, 287 PB, 343 PB |
| 81840 E12 | 245 | PB | 124 | 100 | 3 | plus | 170588629 | | 275547 In1 | | MDS1 | | |
| 81841 E06 | 245 | PB | 491 | 99 | 3 | plus | 170588996 | | 275180 In1 | | MDS1 | | 343 PB |
| 78166 H03 | 149 | PB | 139 | 99.3 | 3 | minus | 170722319 | | 141857 In1 | | MDS1 | | 245 PB, 287 PB |
| 81674 D06 | 245 | PB | 188 | 100 | 3 | plus | 170865957 | 1781 | | | MDS1 | | |
| 77510 A09 | 84 | BM | 69 | 100 | 3 | minus | 170906546 | 42370 | | | MDS1 | | |
| 76856 G05 | 24 | PB | 23 | 100 | 4 | plus | 3115946 | | 2534 In1 | | HD | 36467 bp downstream of GRK4 | |
| 78373 G07 | 149 | PB | 45 | 100 | 4 | minus | 8309381 | 9784 | | | SH3TC1 | 30772 bp upstream of ABLIM2 and | |
| | | | | | | | | | | | | 80182 bp upstream of HTRA3 | |
| 76856 E07 | 24 | PB | 77 | 100 | 4 | plus | 24262823 | 377 | | | DHX15 | | 28 PB |
| 76855 F03 | 24 | PB | 102 | 99.1 | 4 | plus | 41781724 | | 3627 In2 | | TMEM33 | 51753 bp upstream of SLC30A9 | |
| 76855 D11 | 28 | PB | 238 | 100 | 4 | plus | 75287179 | 17654 | | | CXCL3 | 40612 bp downstream of CXCL2 and | |
| | | | | | | | | | | | | 57728 bp upstream of CXCL5 and | |
| | | | | | | | | | | | | 68244 bp upstream of PPBP and | |
| | | | | | | | | | | | | 74467 bp upstream of PF4 | |
| 82775 F12 | 287 | PB | 117 | 100 | 4 | plus | 75546901 | | | 2560 | EPGN | 13054 bp downstream of MTHFD2L | 343 PB |
| | | | | | | | | | | | | and 48994 bp upstream of EREG | |
| 78169 A04 | 84 | PB | 173 | 94.2 | 4 | minus | 123430414 | | | | | no Refseq gene within next 100 kb | |
| 76855 G12 | 28 | PB | 32 | 95.5 | 4 | plus | 134410200 | | | | | no Refseq gene within next 100 kb | |
| 76855 C01 | 24 | PB | 98 | 99 | 4 | plus | 151852923 | | 441331 In32 | | LRBA | | |
| 76856 B04 | 24 | PB | 141 | 99.3 | 4 | minus | 160106703 | | | 65594 | FLJ25371 | | |
| 76855 C10 | 24 | PB | 61 | 10 | 4 | plus | 166859837 | | | 82756 | CPE | | |
| 78373 C08 | 149 | PB | 76 | 100 | 5 | plus | 6765655 | 24965 | | | POLS | 42982 bp downstream of SRD5A1 | |
| | | | | | | | | | | | | and 79498 bp upstream of NSUN2 | |
| 77510 D07 | 84 | PB | 579 | 99.7 | 5 | plus | 13643174 | | | | | no Refseq gene within next 100 kb | |
| 76856 A01 | 24 | PB | 61 | 98.4 | 5 | minus | 25186454 | | | | | no Refseq gene within next 100 kb | |
| 77510 G03 | 84 | PB | 221 | 100 | 5 | plus | 25719953 | | | | | no Refseq gene within next 100 kb | |
| 78168 D07 | 28 | BM | 135 | 100 | 5 | minus | 40714775 | 1014 | | | PTGER4 | 35561 bp downstream of OSRF and | |
| | | | | | | | | | | | | 80464 bp downstream of PRKAA1 | |
| 78168 C01 | 28 | PB | 207 | 98.6 | 5 | plus | 77818082 | | 23785 Ex2 | | LHFPL2 | 7448 bp downstream of SCAMP1 | |
| 78168 C03 | 28 | PB | 60 | 93 | 5 | minus | 79514127 | | 73499 In2 | | C5orf12 | 99266 bp downstream of THBS4 | |
| 77511 A06 | 84 | BM | 31 | 96.8 | 5 | minus | 89739305 | | 2054 Ex2 | | CETN3 | 50473 bp downstream of LOC153364 | |
| | | | | | | | | | | | | and 67177 bp upstream of POLR3G | |
| 76855 D01 | 24 | PB | 186 | 99.5 | 5 | minus | 133867437 | 22260 | | | PHF15 | 91949 bp upstream of MGC13017 | |
| 77511 E06 | 84 | BM | 180 | 99.5 | 5 | minus | 139663413 | 566 | | | PFDN1 | 29200 bp downstream of DTR and | |
| | | | | | | | | | | | | 56566 bp upstream of SLC4A9 and | |
| | | | | | | | | | | | | 59857 bp downstream of ORF1-FL49 | |
| 78168 F01 | 28 | PB | 172 | 97.7 | 5 | plus | 147096341 | | 46104 In1 | | KIAA0555 | 87998 bp downstream of SPINK1 | |
| 77511 D11 | 49 | PB | 74 | 94.3 | 5 | plus | 180604307 | 805 | | | GNB2L1 | | |
| 81841 A09 | 245 | PB | 372 | 99 | 6 | plus | 25151438 | | | | | no Refseq gene within next 100 kb | |
| 78166 G12 | 149 | PB | 50 | 93.5 | 6 | plus | 27770753 | | | | | no Refseq gene within next 100 kb | |
| 78373 A11 | 149 | PB | 340 | 98.9 | 6 | plus | 30578532 | | | 10195 | HLA-E | 43141 bp downstream of GNL1 and | |
| | | | | | | | | | | | | 54110 bp upstream of PRR3 and | |
| | | | | | | | | | | | | 68617 bp upstream of ABCF1 and | |
| | | | | | | | | | | | | 97630 bp downstream of PPP1R10 | |
| 77511 E03 | 84 | BM | 146 | 100 | 6 | minus | 74346626 | | | 13198 | SLC17A5 | 59151 bp upstream of EEF1A1 and | |
| | | | | | | | | | | | | 78730 bp downstream of MTO1 | |
| 81841 A03 | 245 | PB | 299 | 100 | 6 | minus | 90117278 | | 2060 In1 | | UBE2J1 | 17035 bp downstream of RRAGD and | |
| | | | | | | | | | | | | 35605 bp upstream of GABRR2 and | |
| | | | | | | | | | | | | 82373 bp upstream of ANKRD6 | |
| 81674 E08 | 245 | PB | 214 | 100 | 6 | minus | 90979544 | | 83638 In3 | | BACH2 | | |
| 78165 G02 | 149 | PB | 187 | 94.9 | 6 | plus | 143113467 | | | 832 | HIVEP2 | | |
| 78168 B09 | 28 | BM | 245 | 99.6 | 7 | minus | 2137072 | 9732 | | | SNX8 | 30670 bp upstream of EIF3S9 and | |
| | | | | | | | | | | | | 73051 bp downstream of NUDT1 and | |
| | | | | | | | | | | | | 79392 bp upstream of CHST12 and | |
| | | | | | | | | | | | | 81998 bp upstream of FTSJ2 | |
| 81675 B08 | 245 | PB | 172 | 99.5 | 7 | plus | 5634281 | 39748 | | | TRIAD3 | 77301 bp upstream of C7orf28A | |
| 81841 F12 | 245 | PB | 112 | 100 | 7 | minus | 6201881 | | 14515 In2 | | RAC1 | 58944 bp downstream of LOC221955 | |
| | | | | | | | | | | | | and 40051 bp upstream of | |
| | | | | | | | | | | | | MGC12966 and 73629 bp | |
| | | | | | | | | | | | | downstream of KDELR2 | |
| 79273 A08 | 175 | PB | 247 | 99.6 | 7 | minus | 12533138 | | | 29339 | ARL4A | 66671 bp downstream of SCIN | |
| 77510 A07 | 84 | PB | 50 | 100 | 7 | plus | 37254163 | | 7533 In1 | | ELMO1 | | |
| 79273 A01 | 175 | PB | 82 | 100 | 7 | minus | 43578732 | | 7220 In1 | | BLVRA | 36383 bp upstream of FLJ10803 | |
| 81841 G08 | 245 | PB | 323 | 99.7 | 7 | plus | 47805336 | | 36608 In9 | | SUNC1 | 12850 bp upstream of HUS1 and | |
| | | | | | | | | | | | | 44059 bp upstream of PKD1L1 and | |
| | | | | | | | | | | | | 96259 bp upstream of UPP1 | |
| 77510 B03 | 84 | PB | 149 | 99.4 | 7 | plus | 48206948 | | 191844 In34 | | ABCA13 | | |
| 78373 B04 | 149 | PB | 210 | 100 | 7 | plus | 73338718 | | 25947 In1 | | GTF2IRD1 | 73803 bp upstream of CYLN2 and | |
| | | | | | | | | | | | | 52554 bp upstream of WBSCR23 | |
| 77510 C05 | 84 | PB | 99 | 99 | 7 | plus | 77003613 | | 33202 In1 | | RSBN1L | | |
| 77511 A08 | 49 | PB | 41 | 100 | 7 | minus | 87365515 | | 157162 In3 | | ADAM22 | | |
| 78168 A08 | 28 | BM | 41 | 100 | 7 | plus | 104195778 | 52810 | | | MLL5 | 54148 bp downstream of LHFPL3 | |
| 77510 H01 | 84 | PB | 129 | 100 | 7 | plus | 110373760 | | 422538 In3 | | IMMP2L | 14349 bp downstream of LRRN3 | |
| 78168 H08 | 28 | BM | 90 | 98.9 | 7 | minus | 132775594 | | 380510 In1 | | SEC8L1 | | |
| 78166 F02 | 149 | PB | 52 | 100 | 7 | plus | 138334016 | | | 2215 | FLJ12571 | 82296 bp upstream of ZC3HAV1 | |
| 77510 A08 | 84 | PB | 35 | 100 | 7 | minus | 147801706 | 31875 | | | CUL1 | 51107 bp downstream of C7orf33 | |
| 77510 D05 | 84 | PB | 204 | 100 | 8 | minus | 108517885 | | 81545 In1 | | ANGPT1 | | |
| 77510 H04 | 84 | PB | 69 | 100 | 8 | minus | 121151880 | | | 19943 | DEPDC6 | | |
| 77510 G06 | 84 | PB | 74 | 100 | 8 | plus | 145010475 | | | 1425 | EPPK1 | 1425 bp downstream of NRBP2 and | |
| | | | | | | | | | | | | 26950 bp upstream of SIAHBP1 and | |
| | | | | | | | | | | | | 40943 bp upstream of SCRIB and | |
| | | | | | | | | | | | | 75271 bp downstream of PLEC1 | |
| 78165 B12 | 149 | PB | 237 | 99.1 | 9 | plus | 5833135 | 47774 | | | MLANA | | |
| 79275 A04 | 175 | PB | 305 | 99.7 | 9 | plus | 17125960 | | | | | no Refseq gene within next 100 kb | |
| 76855 A06 | 24 | PB | 103 | 99.1 | 9 | minus | 20389646 | | 222804 In5 | | MLLT3 | | |
| 78373 C05 | 149 | PB | 103 | 99.1 | 9 | minus | 33070123 | 3479 | | | SMU1 | 30519 bp downstream of B4GALT1 | |
| | | | | | | | | | | | | and 41061 bp downstream of | |
| | | | | | | | | | | | | DNAJA1 | |
| 76855 B06 | 24 | PB | 188 | 100 | 9 | plus | 65820931 | | | | | no Refseq gene within next 100 kb | |
| 77510 C07 | 84 | PB | 57 | 100 | 9 | minus | 79455484 | | 39052 In4 | | TLE4 | | |
| 78168 A05 | 28 | PB | 71 | 100 | 9 | plus | 94901155 | | 332606 In10 | | C9orf3 | 39736 bp downstream of FANCC | |
| 86611 A03 | 119 | PB | 193 | 100 | 9 | plus | 98918520 | | | 5907 | COL15A1 | 28447 bp upstream of TGFBR1 | |
| 76856 C11 | 28 | PB | 90 | 100 | 9 | minus | 104700931 | | 69060 In6 | | ABCA1 | 85086 bp downstream of NIPSNAP3B | 28 PB |
| 76856 A04 | 24 | PB | 24 | 100 | 9 | minus | 117773153 | | | | | no Refseq gene within next 100 kb | |
| 76855 F01 | 24 | PB | 100 | 97 | 9 | plus | 121199808 | | 12291 In1 | | STOM | 25134 bp downstream of GSN | |
| 77510 B02 | 84 | PB | 90 | 98.9 | 9 | minus | 121236338 | 24239 | | | STOM | 61664 bp downstream of GSN | |
| 78166 B05 | 149 | PB | 82 | 100 | 9 | minus | 122874198 | | 91327 In13 | | RABGAP1 | 2202 bp upstream of GPR21 | |
| 78168 H10 | 28 | BM | 95 | 100 | 9 | plus | 124122407 | | 22484 In1 | | NEK6 | 72892 bp downstream of PSMB7 | |
| 78165 H12 | 149 | PB | 135 | 99.3 | 9 | minus | 124355504 | 6251 | | | NR5A1 | 8753 bp downstream of NR6A1 and | |
| | | | | | | | | | | | | 36571 bp downstream of GPR144 | |
| | | | | | | | | | | | | and 98229 bp upstream of PSMB7 | |
| 76856 A07 | 24 | PB | 542 | 98.4 | 9 | minus | 136885796 | | 2929 In1 | | MGC20262 | 28084 bp upstream of AGPAT2 and | |
| | | | | | | | | | | | | 24018 bp downstream of LCN10 and | |
| | | | | | | | | | | | | 28513 bp downstream of LCN6 and | |
| | | | | | | | | | | | | 42830 bp downstream of EGFL7 | |
| 78168 B02 | 28 | PB | 81 | 100 | 9 | plus | 137490345 | | 103106 In20 | | FLJ20433 | 17805 bp upstream of MGC61598 | |
| | | | | | | | | | | | | and 37416 bp downstream of | |
| | | | | | | | | | | | | FLJ20245 and 46517 bp downstream | |
| | | | | | | | | | | | | of COBRA1 and 66574 bp | |
| | | | | | | | | | | | | downstream of LOC441476 | |
| 86611 A02 | 119 | PB | 89 | 100 | 10 | minus | 6553491 | | 108753 In11 | | PRKCQ | | |
| 77510 D04 | 84 | PB | 216 | 99.6 | 10 | minus | 11694081 | | | | | no Refseq gene within next 100 kb | |
| 76855 E01 | 24 | PB | 162 | 99.4 | 10 | minus | 19977260 | | | | | no Refseq gene within next 100 kb | |
| 86611 E02 | 119 | PB | 71 | 98.6 | 10 | minus | 22663288 | | | 3098 | PCGF4 | 11117 bp upstream of SPAG6 and | |
| | | | | | | | | | | | | 14045 bp downstream of COMMD3 | |
| 77511 F12 | 49 | PB | 120 | 100 | 10 | minus | 30829395 | | | 38628 | MAP3K8 | | |
| 81840 E02 | 245 | PB | 326 | 99.7 | 10 | minus | 49348293 | | 134851 In6 | | ARHGAP22 | 35104 bp downstream of MAPK8 | |
| 78168 B03 | 28 | PB | 203 | 100 | 10 | minus | 50060847 | 67287 | | | C10orf72 | | |
| 78165 B05 | 149 | PB | 23 | 100 | 10 | minus | 70851068 | 4983 | | | TACR2 | 19427 bp downstream of HK1 and | |
| | | | | | | | | | | | | 30164 bp upstream of TM4SF15 | |
| 77511 H05 | 84 | BM | 546 | 99.3 | 10 | plus | 101247649 | 35051 | | | NKX2 | 67313 bp upstream of GOT1 | |
| 81840 G02 | 245 | PB | 498 | 99.8 | 10 | minus | 101281800 | 900 | | | NKX2 | 78472 bp downstream of SLC25A28 | |
| 78165 A12 | 149 | PB | 147 | 98.7 | 10 | minus | 105662154 | | 5802 In2 | | OBFC1 | 55306 bp upstream of SLK | |
| 78373 E02 | 149 | PB | 163 | 100 | 10 | plus | 114494647 | | | 7125 | VTI1A | | |
| 76855 F06 | 24 | PB | 31 | 96.8 | 10 | plus | 116507932 | 73528 | | | ABLIM1 | | |
| 88283 C07 | 343 | PB | 604 | 99.4 | 11 | plus | 9962351 | | 309945 In11 | | SBF2 | | |
| 78169 G07 | 84 | PB | 293 | 99.4 | 11 | plus | 36355244 | | 1113 In1 | | FLJ14213 | 87689 bp upstream of COMMD9 | |
| 78166 E01 | 149 | PB | 190 | 99.5 | 11 | minus | 59582663 | | 1956 In1 | | MS4A3 | 39930 bp upstream of MS4A2 and | |
| | | | | | | | | | | | | 10573 bp downstream of FLJ36198 | |
| 78168 D01 | 28 | PB | 71 | 98.6 | 11 | plus | 65878220 | 6549 | | | B3GNT6 | 9088 bp upstream of BRMS1 and | |
| | | | | | | | | | | | | 8349 bp downstream of SLC29A2 and | |
| | | | | | | | | | | | | 17644 bp upstream of RIN1 and | |
| | | | | | | | | | | | | 37129 bp upstream of CD248 | |
| 78373 B07 | 149 | PB | 197 | 100 | 11 | plus | 70840909 | 956 | | | NADSYN1 | 3682 bp upstream of DHCR7 and | |
| | | | | | | | | | | | | 28861 bp upstream of FLJ42102 and | |
| | | | | | | | | | | | | 75086 bp upstream of UHSKerB and | |
| | | | | | | | | | | | | 96205 bp upstream of KRN1 | |
| 77510 H12 | 84 | BM | 183 | 99.5 | 11 | plus | 72128107 | 17079 | | | CENTD2 | 15425 bp downstream of STARD10 | |
| | | | | | | | | | | | | and 65047 bp upstream of PDE2A | |
| 78169 C05 | 84 | PB | 81 | 98.8 | 11 | minus | 95626354 | | 89638 In1 | | MAML2 | | |
| 78168 A04 | 28 | PB | 277 | 99.7 | 11 | minus | 117776969 | 345 | | | ATP5L | 1835 bp downstream of UBE4A and | |
| | | | | | | | | | | | | 32097 bp downstream of MGC13053 | |
| | | | | | | | | | | | | and 35446 bp upstream of MLL and | |
| | | | | | | | | | | | | 36185 bp upstream of FLJ11783 | |
| 78165 B08 | 149 | PB | 28 | 100 | 11 | minus | 128051158 | 18041 | | | FLI1 | | |
| 82775 D11 | 287 | PB | 307 | 100 | 12 | plus | 2482645 | | 449920 In7 | | CACNA1C | | |
| 76857 C05 | 28 | BM | 158 | 99.4 | 12 | minus | 2483055 | | 450330 In7 | | CACNA1C | | |
| 76855 B04 | 24 | PB | 72 | 98.7 | 12 | minus | 13036334 | | 8156 In1 | | HEBP1 | 41749 bp of GPRC5D and 78479 bp | |
| | | | | | | | | | | | | downstream of RAI3 and 91427 bp | |
| | | | | | | | | | | | | downstream of GSG1 | |
| 78169 D04 | 84 | PB | 101 | 100 | 12 | minus | 29271003 | | 3138 In1 | | MLSTD1 | | |
| 78169 H05 | 84 | PB | 22 | 100 | 12 | plus | 30739843 | | 175 Ex1 | | IPO8 | 13910 bp downstream of C1QDC1 | |
| 78169 C01 | 49 | PB | 66 | 100 | 12 | plus | 44406271 | 3616 | | | ARID2 | | |
| 79273 G07 | 175 | PB | 252 | 98.6 | 12 | minus | 44855105 | | | 12746 | SLC38A1 | | |
| 79273 C12 | 175 | PB | 30 | 100 | 12 | minus | 50932924 | | | 3948 | KRT7 | 33042 bp downstream of KRTHB1 | |
| | | | | | | | | | | | | and 48992 bp upstream of KRTHB6 | |
| | | | | | | | | | | | | and 61428 bp downstream of | |
| | | | | | | | | | | | | KRTHB3 and 67124 bp upstream of | |
| | | | | | | | | | | | | LOC144501 | |
| 82775 B06 | 287 | PB | 170 | 100 | 12 | plus | 60943883 | | 3429 In1 | | USP15 | 71065 bp upstream of FAM19A2 | |
| 78169 D12 | 84 | BM | 93 | 100 | 12 | minus | 61115371 | 31496 | | | KIAA1040 | 29206 bp downstream of USP15 | |
| 78165 G12 | 149 | PB | 115 | 99.2 | 12 | plus | 63766210 | | 35149 In2 | | WIF1 | 83431 bp upstream of MAN1 | |
| 76856 C07 | 24 | PB | 52 | 100 | 12 | plus | 64849040 | | 991 In1 | | CGI-119 | 20244 bp upstream of IRAK3 and | 28 PB |
| | | | | | | | | | | | | 38240 bp upstream of MGC14817 | |
| 77511 H11 | 49 | PB | 189 | 100 | 12 | minus | 88206706 | | | 37601 | DUSP6 | | |
| 78165 B09 | 149 | PB | 169 | 99.5 | 12 | minus | 100799663 | | 2094 In1 | | FLJ11259 | 72563 bp upstream of MGC4170 | |
| 78168 B08 | 28 | BM | 219 | 99.1 | 12 | minus | 107210404 | | 25149 In2 | | CMKLR1 | 63626 bp downstream of KIAA0789 | |
| 77511 B11 | 49 | PB | 91 | 100 | 12 | plus | 107758711 | 4894 | | | SSH1 | 17622 bp upstream of DAO and | |
| | | | | | | | | | | | | 48417 bp downstream of | |
| | | | | | | | | | | | | DKFZp761H039 | |
| 78169 G12 | 84 | BM | 75 | 100 | 12 | minus | 112142036 | | 20047 In2 | | TPCN1 | 20436 bp upstream of IQCD and | |
| | | | | | | | | | | | | 57256 bp downstream of SLC24A6 | |
| | | | | | | | | | | | | and 49197 bp downstream of | |
| | | | | | | | | | | | | FLJ14827 and 56032 bp upstream of | |
| | | | | | | | | | | | | DDX54 | |
| 77510 G08 | 84 | PB | 459 | 99.8 | 12 | plus | 115462706 | | | 26660 | FLJ42957 | | |
| 76856 H01 | 24 | PB | 24 | 100 | 12 | plus | 117230472 | | 42799 In1 | | JIK | 61204 bp upstream of SDS3 | |
| 78373 C09 | 149 | PB | 277 | 99.7 | 13 | minus | 27528565 | | 44138 In4 | | FLT3 | 87248 bp upstream of CDX2 | |
| 78169 A07 | 84 | PB | 107 | 100 | 13 | plus | 44869659 | 56362 | | | TPT1 | 67413 bp upstream of COG3 | |
| 78165 E04 | 149 | PB | 23 | 100 | 13 | plus | 48355056 | 93884 | | | FNDC3 | | |
| 78166 D05 | 149 | PB | 54 | 98.2 | 13 | plus | 66918583 | | | | | no Refseq gene within next 100 kb | |
| 76856 D06 | 24 | PB | 142 | 100 | 14 | plus | 31738256 | | | 44161 | ARHGAP5 | | |
| 79275 C09 | 175 | PB | 67 | 100 | 14 | minus | 49509663 | | | 78179 | ARF6 | | |
| 78168 D08 | 28 | BM | 82 | 100 | 14 | plus | 66043883 | 1106 | | | GPHN | 8860 bp downstream of MGC88374 | |
| 76855 G07 | 24 | PB | 75 | 100 | 14 | plus | 76537063 | | | 23578 | C14orf4 | 97268 bp downstream of KIAA1737 | |
| 76855 D03 | 24 | PB | 145 | 98.7 | 14 | minus | 80937010 | 2330 | | | STN2 | 70636 bp downstream of SEL1L | |
| 76855 F02 | 24 | PB | 45 | 100 | 14 | plus | 89161678 | | | | | no Refseq gene within next 100 kb | |
| 76857 A04 | 84 | BM | 88 | 100 | 14 | plus | 101410689 | | 64764 In2 | | PPP2R5C | 90048 bp upstream of DNCH1 | |
| 77510 B04 | 84 | PB | 114 | 100 | 14 | plus | 102591936 | | 1616 In1 | | CDC42BPB | 70481 bp upstream of TNFAIP2 | |
| 77511 A03 | 84 | BM | 130 | 100 | 15 | plus | 35846410 | | | | | no Refseq gene within next 100 kb | |
| 78169 F05 | 84 | PB | 59 | 100 | 15 | plus | 46891093 | 574 | | | CEP152 | 12134 bp downstream of RALP and | |
| | | | | | | | | | | | | 66489 bp upstream of CRI1 | |
| 77510 A02 | 84 | PB | 134 | 99.3 | 15 | plus | 62648517 | | | | | no Refseq gene within next 100 kb | |
| 81840 E01 | 245 | PB | 188 | 99.5 | 15 | minus | 62783064 | 557 | | | OAZ2 | | |
| 86611 A04 | 119 | PB | 91 | 100 | 15 | plus | 72032059 | | | 26217 | LOXL1 | 30555 bp downstream of STOML1 | |
| | | | | | | | | | | | | and 42008 bp upstream of PML and | |
| | | | | | | | | | | | | 63453 bp downstream of TBC1D21 | |
| 82776 H06 | 287 | PB | 145 | 95.8 | 15 | minus | 83890538 | | 165663 In5 | | AKAP13 | | |
| 77510 H03 | 84 | PB | 155 | 99.4 | 15 | minus | 96436854 | | | | | no Refseq gene within next 100 kb | |
| 82774 F03 | 287 | PB | 181 | 100 | 15 | plus | 99601313 | | 8336 In1 | | CHSY1 | 27424 bp downstream of SELS and | |
| | | | | | | | | | | | | 37925 bp downstream of SNRPA1 | |
| | | | | | | | | | | | | and 60343 bp downstream of PCSK6 | |
| 76855 B05 | 24 | PB | 204 | 98.6 | 16 | minus | 1638435 | | 16174 In4 | | CRAMP1L | 29844 bp upstream of C16orf34 and | |
| | | | | | | | | | | | | 41134 bp upstream of KIAA0590 and | |
| | | | | | | | | | | | | 57787 bp upstream of MAPK8IP3 and | |
| | | | | | | | | | | | | 92855 bp downstream of C16orf30 | |
| 76857 B12 | 35 | PB | 95 | 100 | 16 | plus | 10879629 | | 1089 In1 | | MHC2TA | 50622 bp downstream of DEXI | |
| 76856 A12 | 28 | PB | 181 | 98.9 | 16 | minus | 14942713 | | 3912 In1 | | NPIP | 33774 bp upstream of KIAA0251 and | |
| | | | | | | | | | | | | 45199 bp downstream of NOMO1 | |
| 77510 F11 | 84 | BM | 72 | 100 | 16 | plus | 29572198 | 9882 | | | SPN | 25744 bp upstream of QPRT and | |
| | | | | | | | | | | | | 39659 bp upstream of LAT1-3TM and | |
| | | | | | | | | | | | | 89091 bp downstream of FLJ35681 | |
| 76857 B04 | 84 | BM | 148 | 100 | 16 | plus | 51016911 | | | | | no Refseq gene within next 100 kb | |
| 78169 B10 | 84 | BM | 74 | 100 | 16 | minus | 54071488 | | 884 In1 | | MMP2 | 28967 bp upstream of FLJ20481 and | |
| | | | | | | | | | | | | 87605 bp upstream of CAPNS2 | |
| 86611 E07 | 119 | PB | 89 | 98.9 | 16 | minus | 54887735 | | 104086 In2 | | GNAO1 | | |
| 76855 C04 | 24 | PB | 88 | 100 | 16 | minus | 56283590 | 2637 | | | DKFZp434I099 | 2801 bp downstream of GPR97 and | |
| | | | | | | | | | | | | 27145 bp downstream of GPR56 and | |
| | | | | | | | | | | | | 45028 bp upstream of KATNB1 and | |
| | | | | | | | | | | | | 66042 bp downstream of KIFC3 | |
| 78169 F12 | 84 | BM | 209 | 99.6 | 16 | plus | 69019240 | | 11252 In1 | | SIAT4B | 26759 bp upstream of FUK and | |
| | | | | | | | | | | | | 52738 bp downstream of COG4 and | |
| | | | | | | | | | | | | 54460 bp downstream of DOX19L | |
| | | | | | | | | | | | | and 94010 bp downstream of DOX19 | |
| 77510 A04 | 84 | PB | 183 | 98.8 | 16 | minus | 80389163 | | 18732 In2 | | PLCG2 | 86297 bp downstream of CMIP | |
| 77510 D03 | 84 | PB | 131 | 96.1 | 17 | plus | 1337791 | | 4954 In1 | | MYO1C | 6831 bp downstream of SKIP and | |
| | | | | | | | | | | | | 31497 bp upstream of CRK and | |
| | | | | | | | | | | | | 30244 bp downstream of PITPNA and | |
| | | | | | | | | | | | | 86655 bp downstream of SLC43A2 | |
| 78165 D06 | 149 | PB | 73 | 98.7 | 17 | plus | 1939585 | | 214184 In12 | | C17orf31 | 30706 bp downstream of HIC1 and | |
| | | | | | | | | | | | | 46111 bp downstream of OVCA2 and | |
| | | | | | | | | | | | | 46116 bp downstream of DPH2L1 | |
| 79275 C06 | 175 | PB | 207 | 100 | 17 | plus | 15629919 | | | 39718 | MGC51025 | 67956 bp downstream of ZNF286 | |
| 76855 D07 | 24 | PB | 132 | 99.3 | 17 | plus | 22704253 | | | 39481 | WSB1 | | |
| 78168 A07 | 28 | BM | 120 | 100 | 17 | plus | 24094209 | 964 | | | TRAF4 | 289 bp downstream of NEK8 and | |
| | | | | | | | | | | | | 16856 bp downstream of LOC116238 | |
| | | | | | | | | | | | | and 18709 bp downstream of RPL23A | |
| | | | | | | | | | | | | and 13647 bp downstream of | |
| | | | | | | | | | | | | FLJ10700 | |
| 77510 F04 | 84 | PB | 82 | 98.8 | 17 | minus | 25072509 | | 208635 In2 | | SSH2 | | |
| 78373 D12 | 149 | PB | 163 | 98.2 | 17 | plus | 27240091 | | 12751 In7 | | HCA66 | 29722 bp upstream of HSA272196 | |
| | | | | | | | | | | | | and 48094 bp upstream of SUZ12 | |
| 78169 G09 | 84 | BM | 154 | 99.4 | 17 | plus | 30406494 | | 33913 In1 | | RFFL | 45021 bp downstream of RAD51L3 | |
| | | | | | | | | | | | | and 50558 bp downstream of LIG3 | |
| | | | | | | | | | | | | and 66299 bp upstream of | |
| | | | | | | | | | | | | DKFZp434H2215 and 75989 bp | |
| | | | | | | | | | | | | downstream of FLJ10458 | |
| 78168 B10 | 28 | BM | 44 | 97.8 | 17 | minus | 32924645 | | 525 In1 | | DUSP14 | 12160 bp downstream of TADA2L | |
| | | | | | | | | | | | | and 27401 bp downstream of | |
| | | | | | | | | | | | | AP1GP1 and 83630 bp upstream of | |
| | | | | | | | | | | | | ACACA | |
| 76857 C11 | 35 | PB | 124 | 99.2 | 17 | plus | 33254417 | 75235 | | | TCF2 | | |
| 77510 A12 | 84 | BM | 75 | 100 | 17 | minus | 44766072 | | 28762 In1 | | ZNF652 | 70347 bp downstream of PHB and | |
| | | | | | | | | | | | | 77262 bp downstream of FLJ40194 | |
| 78168 G07 | 28 | BM | 123 | 100 | 17 | plus | 50525888 | | | 48752 | STXBP4 | | |
| 78166 F03 | 149 | PB | 97 | 100 | 17 | plus | 50599958 | 97417 | | | HLF | | |
| 79275 A05 | 175 | PB | 148 | 100 | 17 | minus | 52887031 | | 198101 In3 | | MSI2 | | |
| 76857 E01 | 84 | BM | 101 | 99.1 | 17 | minus | 55219365 | | 79554 In7 | | VMP1 | 72268 bp downstream of TUBD1 and | |
| | | | | | | | | | | | | 79727 bp upstream of BIT1 and | |
| | | | | | | | | | | | | 92111 bp downstream of CLTC | |
| 78166 E02 | 149 | PB | 206 | 100 | 17 | minus | 62686728 | 14954 | | | HELZ | 77767 bp downstream of PSMD12 | |
| 81674 D08 | 245 | PB | 100 | 100 | 17 | plus | 64924063 | | 1629 In1 | | MAP2K6 | 89178 bp upstream of ABCA5 | |
| 81840 G03 | 245 | PB | 226 | 99.6 | 17 | minus | 70028583 | | | 326 | TREM5 | 20259 bp downstream of CD300C | |
| | | | | | | | | | | | | and 36057 bp downstream of | |
| | | | | | | | | | | | | CD300A and 64069 bp upstream of | |
| | | | | | | | | | | | | FLJ31882 and 73422 bp downstream | |
| | | | | | | | | | | | | of GPRC5C | |
| 77510 G01 | 84 | PB | 97 | 97 | 17 | plus | 70247026 | | 2070 In2 | | RAB37 | 9353 bp upstream of SLC9A3R1 and | |
| | | | | | | | | | | | | 26323 bp upstream of NKIR and | |
| | | | | | | | | | | | | 31257 bp downstream of EBSP and | |
| | | | | | | | | | | | | 37243 bp upstream of FLJ20255 | |
| 78166 F04 | 149 | PB | 73 | 98.7 | 17 | minus | 72595998 | 52619 | | | SEC14L1 | | |
| 79275 A06 | 175 | PB | 210 | 99.1 | 17 | minus | 72929730 | | 101986 In2 | | SEPT9 | | |
| 76856 D10 | 24 | PB | 117 | 96.5 | 17 | plus | 73737231 | | | 4859 | BIRC5 | 42505 bp upstream of TK1 and | |
| | | | | | | | | | | | | 56627 bp downstream of SYNGR2 | |
| | | | | | | | | | | | | and 88198 bp downstream of EVER2 | |
| | | | | | | | | | | | | and 97148 bp upstream of EVER1 | |
| 78166 B06 | 149 | PB | 75 | 100 | 17 | plus | 74281828 | | 8143 In1 | | PSCD1 | 13316 bp downstream of USP36 and | |
| | | | | | | | | | | | | 78828 bp downstream of TIMP2 | |
| 78168 F10 | 28 | BM | 80 | 100 | 17 | plus | 78000768 | 4417 | | | MGC4368 | 6964 bp downstream of FLJ23825 | |
| | | | | | | | | | | | | and 31017 bp upstream of FLJ22222 | |
| | | | | | | | | | | | | and 38662 bp upstream of NARF and | |
| | | | | | | | | | | | | 70115 bp upstream of FOXK2 | |
| 78169 F04 | 84 | PB | 182 | 99.5 | 18 | minus | 9093169 | | 444 In1 | | NDUFV2 | 33632 bp upstream of ANKRD12 | |
| 76855 C02 | 24 | PB | 93 | 99 | 18 | minus | 42038619 | 11174 | | | C18orf25 | 76323 bp downstream of CCDC5 and | |
| | | | | | | | | | | | | 100422 bp upstream of ATP5A1 | |
| 78169 A03 | 49 | PB | 191 | 97.7 | 18 | plus | 53646958 | 96921 | | | ATP8B1 | | |
| 78165 A05 | 149 | PB | 30 | 96.7 | 18 | plus | 61573082 | | 4594 In1 | | CDH7 | | |
| 79275 F06 | 175 | PB | 243 | 100 | 18 | plus | 72553713 | | | 19133 | FLJ44881 | | 245 PB |
| 77510 A06 | 84 | PB | 84 | 100 | 18 | minus | 75364363 | | 107603 In8 | | NFATC1 | | |
| 78168 E02 | 28 | PB | 31 | 100 | 19 | plus | 2240345 | 7170 | | | C19orf35 | 15858 bp downstream of OAZ1 and | |
| | | | | | | | | | | | | 32177 bp downstream of LSM7 and | |
| | | | | | | | | | | | | 34001 bp upstream of FLJ32416 and | |
| | | | | | | | | | | | | 37274 bp downstream of AMH | |
| 77510 F12 | 84 | BM | 448 | 99.2 | 19 | minus | 2503490 | | 150173 In3 | | GNG7 | 74235 bp downstream of GADD45B | |
| | | | | | | | | | | | | and 95532 bp upstream of LMNB2 | |
| 78373 E10 | 149 | PB | 37 | 100 | 19 | plus | 3736609 | | 805 In1 | | MATK | 13390 bp upstream of MGC15631 | |
| | | | | | | | | | | | | and 18054 bp downstream of | |
| | | | | | | | | | | | | MRPL54 and 23936 bp upstream of | |
| | | | | | | | | | | | | APBA3 and 34927 bp downstream of | |
| | | | | | | | | | | | | TPJ3 | |
| 76855 B09 | 24 | PB | 62 | 100 | 19 | plus | 6679790 | 8130 | | | C3 | 10917 bp upstream of TRIP10 and | |
| | | | | | | | | | | | | 23385 bp downstream of SH2D3A | |
| | | | | | | | | | | | | and 58191 bp upstream of TNFSF14 | |
| | | | | | | | | | | | | and 43932 bp upstream of VAV1 | |
| 78166 C05 | 149 | PB | 118 | 100 | 19 | minus | 7487497 | | 422 In1 | | ZNF358 | 6015 bp upstream of MCOLN1 and | |
| | | | | | | | | | | | | 17578 bp upstream of NTE and | |
| | | | | | | | | | | | | 8161 bp downstream of FLJ35784 | |
| | | | | | | | | | | | | and 27592 bp upstream of PEX11G | |
| 78169 C12 | 84 | BM | 213 | 100 | 19 | minus | 13075756 | 1075 | | | LYL1 | 960 bp downstream of FLJ20244 and | |
| | | | | | | | | | | | | 5146 bp downstream of NFIX and | |
| | | | | | | | | | | | | 18621 bp upstream of BTBD14B and | |
| | | | | | | | | | | | | 40468 bp downstream of STX10 | |
| 78168 C07 | 28 | BM | 195 | 100 | 19 | minus | 13075774 | 1093 | | | LYL1 | 942 bp downstream of FLJ20244 and | |
| | | | | | | | | | | | | 5164 bp downstream of NFIX and | |
| | | | | | | | | | | | | 14335 bp upstream of BTBD14B and | |
| | | | | | | | | | | | | 40450 bp downstream of STX10 | |
| 78373 E03 | 149 | PB | 59 | 100 | 19 | minus | 16858930 | 1896 | | | F2RL3 | 5828 bp downstream of CPAMD8 and | |
| | | | | | | | | | | | | 6766 bp downstream of SIN3B and | |
| | | | | | | | | | | | | 69169 bp downstream of LOC284434 | |
| 86758 B09 | 343 | PB | 58 | 100 | 19 | minus | 17996024 | | | 10115 | ARRDC2 | 25094 bp downstream of KCNN1 and | |
| | | | | | | | | | | | | 10119 bp downstream of ARRDC2 | |
| | | | | | | | | | | | | (isoform2) and 35347 bp downstream | |
| | | | | | | | | | | | | of IL12RB1 (isoform1) and 46349 bp | |
| | | | | | | | | | | | | downstream of IL12RB1 (isoform2) | |
| | | | | | | | | | | | | and 80241 bp downstream of | |
| | | | | | | | | | | | | LOC115098 | |
| 78169 D07 | 84 | PB | 132 | 100 | 19 | plus | 19838499 | 11790 | | | ZNF253 | 34288 bp upstream of ZNF505 | |
| 77511 B05 | 84 | BM | 117 | 99.2 | 19 | plus | 21587709 | | | 75144 | ZNF429 | | |
| 81673 C09 | 245 | PB | 52 | 100 | 19 | plus | 33120381 | | | | | no Refseq gene within next 100 kb | |
| 77510 F03 | 84 | PB | 272 | 99.3 | 19 | minus | 33652248 | | | | | no Refseq gene within next 100 kb | |
| 78165 F06 | 149 | PB | 30 | 100 | 19 | plus | 40180368 | 2718 | | | KIAA1533 | 33006 bp upstream of SCN1B and | |
| | | | | | | | | | | | | 37324 bp upstream of FLJ38451 and | |
| | | | | | | | | | | | | 42886 bp upstream of HPN and | |
| | | | | | | | | | | | | 52459 bp downstream of ZNF30 | |
| 78169 B07 | 84 | PB | 107 | 99.1 | 19 | minus | 40925734 | | 2412 In5 | | U2AF1L3 | 543 bp upstream of FLJ22573 and | |
| | | | | | | | | | | | | 2600 bp upstream of PEN2 and | |
| | | | | | | | | | | | | 5618 bp upstream of F25965 and | |
| | | | | | | | | | | | | 4115 bp downstream of MLL4 | |
| 76855 A01 | 24 | PB | 171 | 98.8 | 19 | minus | 44519917 | 1906 | | | GMFG | 38765 bp downstream of IL29 and | |
| | | | | | | | | | | | | 48195 bp downstream of PD2 and | |
| | | | | | | | | | | | | 53886 bp upstream of IXL and | |
| | | | | | | | | | | | | 69410 bp upstream of ZFP36 | |
| 78168 A02 | 28 | PB | 105 | 99.1 | 19 | plus | 63756804 | | 1363 In2 | | BC-2 | 2088 bp downstream of UBE2M and | |
| | | | | | | | | | | | | 2910 bp downstream of TRIM28 and | |
| | | | | | | | | | | | | 8293 bp downstream of ZNF42 and | |
| | | | | | | | | | | | | 21835 bp upstream of MGC2752 | |
| 78168 B04 | 28 | PB | 187 | 100 | 20 | plus | 5007107 | | | 21382 | C20orf30 | 36492 bp downstream of PCNA and | |
| | | | | | | | | | | | | 48375 bp upstream of CDS2 and | |
| | | | | | | | | | | | | 68168 bp upstream of SLC23A2 | |
| 81675 F04 | 245 | PB | 24 | 100 | 20 | minus | 8531384 | | 470088 In3 | | PLCB1 | | |
| 79275 E07 | 175 | PB | 149 | 99.4 | 20 | plus | 23080534 | 19276 | | | LOC200261 | 65557 bp upstream of C1QR1 | 245 PB |
| 76856 G02 | 24 | PB | 112 | 97.5 | 20 | plus | 30591949 | 57100 | | | C20orf112 | 92374 bp downstream of FLJ33706 | 28 PB |
| 76856 F11 | 28 | PB | 34 | 100 | 20 | plus | 42734313 | 20523 | | | ADA | 42986 bp upstream of WISP2 and | |
| | | | | | | | | | | | | 53222 bp downstream of PKIG and | |
| | | | | | | | | | | | | 73589 bp upstream of KCNK15 and | |
| | | | | | | | | | | | | 79550 bp downstream of RIMS4 | |
| 79275 G10 | 175 | PB | 47 | 100 | 20 | plus | 46801890 | | 75937 In1 | | PREX1 | | |
| 76856 E01 | 24 | PB | 115 | 100 | 21 | minus | 15689478 | | | | | no Refseq gene within next 100 kb | 28 PB |
| 78169 E09 | 84 | BM | 60 | 95 | 21 | minus | 16491343 | | 2773 In1 | | C21orf34 | | |
| 82774 A01 | 287 | PB | 179 | 98.9 | 21 | minus | 18382885 | | | | | no Refseq gene within next 100 kb | |
| 78165 E01 | 149 | PB | 150 | 98.7 | 21 | plus | 25782021 | 56137 | | | C21orf42 | 97820 bp downstream of MRPL39 | |
| 78165 A07 | 149 | PB | 128 | 99.3 | 21 | minus | 38676544 | | | 837 | ERG | 80930 bp downstream of KCNJ15 | 175 PB, 245 PB |
| 78169 A10 | 84 | BM | 282 | 99.3 | 21 | plus | 38740822 | | 51445 In1 | | ERG | | |
| 86758 H06 | 343 | PB | 233 | 100 | 21 | plus | 38741710 | | 50557 In1 | | ERG | | |
| 78166 G02 | 149 | PB | 125 | 100 | 21 | plus | 42522618 | | 13362 In2 | | ABCG1 | 82615 bp downstream of TFF3 and | |
| | | | | | | | | | | | | 86444 bp downstream of UMODL1 | |
| 78168 D05 | 28 | PB | 182 | 99.5 | 22 | minus | 26496663 | | 25377 In1 | | MN1 | 75549 bp downstream of PITPNB | |
| 78373 F02 | 149 | PB | 164 | 99.4 | 22 | minus | 26523220 | 1180 | | | MN1 | 48992 bp downstream of PITPNB | |
| 78166 E10 | 149 | PB | 56 | 100 | 22 | minus | 27161115 | | | | | no Refseq gene within next 100 kb | |
| 77511 G02 | 84 | BM | 55 | 100 | 22 | minus | 28821085 | | 20078 In2 | | HORMAD2 | 72662 bp downstream of MTMR3 | |
| 78169 A02 | 49 | PB | 63 | 96.9 | 22 | plus | 38828468 | | | 77925 | LOC113826 | | |
| 79273 C06 | 175 | PB | 178 | 98.9 | 22 | plus | 42202141 | 21279 | | | C22orf1 | 47414 bp downstream of FLJ23588 | |
| 78168 F08 | 28 | BM | 249 | 99.6 | X | plus | 43961020 | 1848 | | | EFHC2 | | |
| 76856 C03 | 24 | PB | 31 | 100 | X | minus | 99789810 | | 2833 In1 | | SYLT4 | 57375 bp downstream of SRPX2 and | |
| | | | | | | | | | | | | 91719 bp upstream of CSTF2 and | |
| | | | | | | | | | | | | 91871 bp upstream of TM4SF8 | |
| 77510 H06 | 84 | PB | 28 | 100 | X | plus | 134581506 | 10228 | | | MGC27005 | | |
| 78373 F09 | 149 | PB | 295 | 99.7 | X | minus | 135010098 | | 54898 In2 | | FHL1 | | |
| 77510 H08 | 84 | PB | 56 | 98.3 | X | minus | 135035866 | | | 16838 | FHL1 | 94710 bp upstream of GPR12 | |
| 81673 E11 | 245 | PB | 91 | 100 | X | plus | 153525839 | | 17197 In1 | | GAB3 | 29015 bp upstream of DKC1 and | |
| | | | | | | | | | | | | 44833 bp downstream of MPP1 and | |
| | | | | | | | | | | | | 81883 bp upstream of CTAG2 | |
| 79275 A07 | 175 | PB | 52 | 100 | X, Y | plus | 1415671 | 19372 | | | CSF2RA | | |
| 86611 A08 | 119 | PB | 131 | 100 | X, Y | minus | 302157 | | 15470 In1 | | PPP2R3B | | |

### Analysis of Insertion Location Changes Over Time Using LAM-PCR

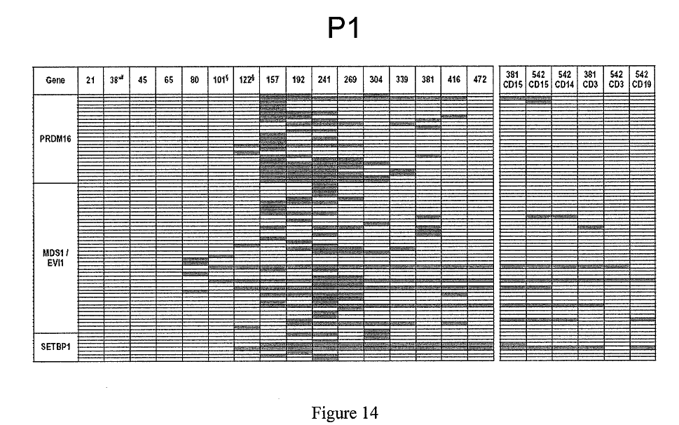

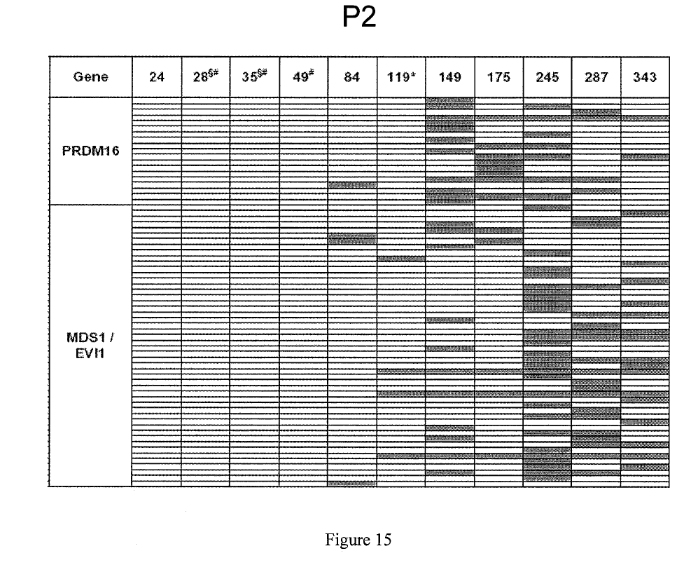

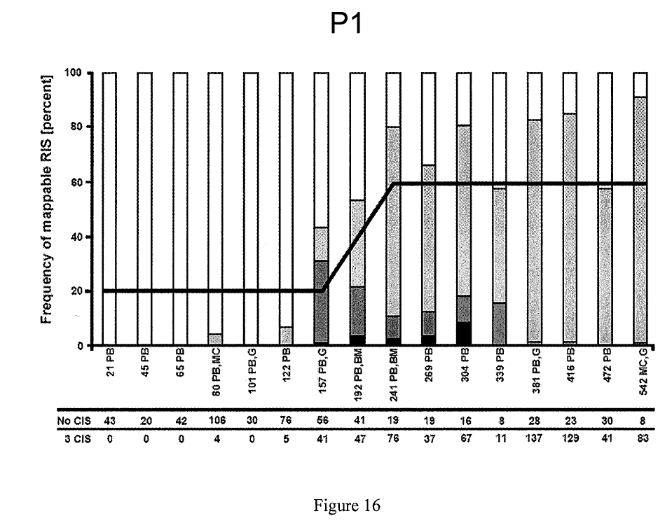

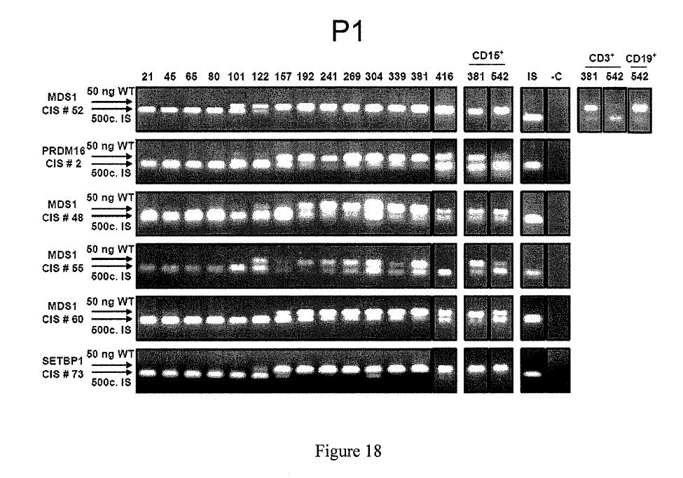

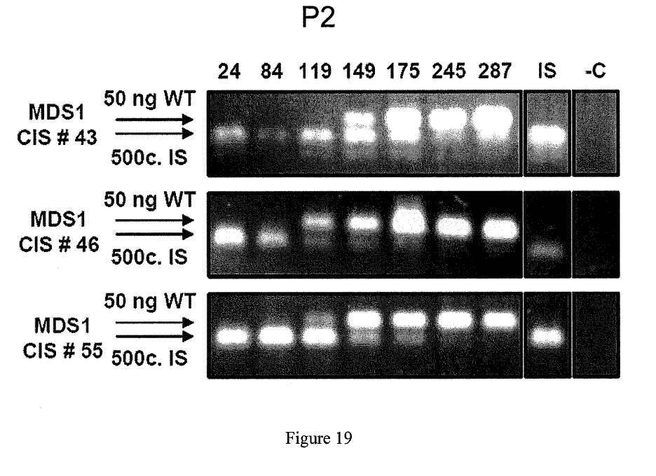

To assess the overall contribution of PR domain (PR+) clones and SETBP1 clones to myelopoiesis over time, the retrieval frequency of unique insertions in shot-gun cloned and sequenced LAM-PCR amplicons was determined from the two patients. After the first appearance of PR+ and SETBP1 RIS on day 84 (patient P1) and day 80 (patient P2), their proportional contribution successively increased to more than 80% of insertions retrieved from circulating transduced cells within the next 100-150 days. The levels of contribution from the 3 CIS then stabilized, matching the 3- to 4-fold expansion of gene-modified myelopoiesis, and plateaued without abnormal elevation of total leukocyte or neutrophil numbers (FIGS. 16,17). Individual clones showed substantial differences in their quantitative myeloid contribution over time. PCR tracking (as described in Example 4) of the 3 CIS clones confirmed the presence of some insertions that were only detectable in one sample as well as other more dominant clones that persistently accounted for substantial percentages of peripheral blood myeloid cells without evidence of exhaustion (FIGS. 14, 15 and Table 2). Dominant clones were further analyzed by quantitative-competitive (QC) PCR (as described in Example 5), which confirmed their stability for a period of between 5 to 14 months after the initial expansion (FIGS. 18, 19).

TABLE 2a

| | Sequence | | | Vector | UCSC | | | | | | | | |
|---|---|---|---|---|---|---|---|---|---|---|---|---|---|
| CIS# | Identity | Gene | Chromosome | Orientation | Locus | Track | 21 | 38*^{#} | 45 | 65 | 80 | 101^{§} | 122^{§} |
| 1 | 75916 B11 | PRDM16 | 1 | same | 3018470 | | | | | | | | |
| 2 | 75917 D12 | PRDM16 | 1 | same | 3109854 | T, Q | | | | | | | |
| 3 | 76778 G06 | PRDM16 | 1 | reverse | 3110903 | | | | | | | | |
| 4 | 76778 D03 | PRDM16 | 1 | reverse | 3111126 | | | | | | | | |
| 5 | 76777 C11 | PRDM16 | 1 | reverse | 3111239 | | | | | | | | |
| 6 | 76777 B04 | PRDM16 | 1 | reverse | 3111424 | T | | | | | | | |
| 7 | 76778 G12 | PRDM16 | 1 | same | 3122160 | T | | | | | | | |
| 8 | 77512 G08 | PRDM16 | 1 | same | 3122190 | | | | | | | | |
| 9 | | PRDM16 | 1 | same | 3122745 | | | | | | | | |
| 10 | | PRDM16 | 1 | same | 3122959 | | | | | | | | |
| 11 | | PRDM16 | 1 | same | 3124251 | | | | | | | | |
| 12 | | PRDM16 | 1 | same | 3122428 | | | | | | | | |
| 13 | | PRDM16 | 1 | same | 3123854 | | | | | | | | |
| 14 | | PRDM16 | 1 | same | 3123893 | | | | | | | | |
| 15 | 75523 G10 | PRDM16 | 1 | same | 3123676 | T | | | | | | | L |
| 16 | 76778 G04 | PRDM16 | 1 | same | 3123793 | T | | | | | | | |
| 17 | 76774 E10 | PRDM16 | 1 | reverse | 3123869 | | | | | | | | L |
| 18 | | PRDM16 | 1 | same | 3123903 | | | | | | | | |
| 19 | 75916 F03 | PRDM16 | 1 | same | 3123915 | | | | | | | | |
| 20 | 76777 B11 | PRDM16 | 1 | same | 3123949 | T | | | | | | | |
| 21 | 75917 B07 | PRDM16 | 1 | same | 3123975 | T | | | | | | | |
| 22 | 75917 G07 | PRDM16 | 1 | same | 3124326 | T | | | | | | | |
| 23 | 76778 C05 | PRDM16 | 1 | same | 3124344 | T | | | | | | | |
| 24 | 76778 B07 | PRDM16 | 1 | same | 3124391 | T | | | | | | | |
| 25 | 78372 D05 | PRDM16 | 1 | same | 3124446 | | | | | | | | |
| 26 | 77048 G07 | EVI1 | 3 | same | 170308560 | | | | | | | | |
| 27 | 76771 H02 | EVI1 | 3 | same | 170337950 | T | | | | | | | |
| 28 | 77110 H11 | EVI1 | 3 | reverse | 170338708 | | | | | | | | |
| 29 | 77110 D02 | EVI1 | 3 | same | 170339175 | T | | | | | | | |
| 30 | 75916 D12 | EVI1 | 3 | same | 170339748 | T | | | | | | | |
| 31 | 77048 E02 | EVI1 | 3 | reverse | 170340583 | T | | | | | | | |
| 32 | 76776 C04 | EVI1 | 3 | same | 170340730 | | | | | | | | |
| 33 | 75917 C09 | EVI1 | 3 | same | 170342916 | | | | | | | | |
| 34 | 75916 F04 | EVI1 | 3 | same | 170343812 | T | | | | | | | |
| 35 | 81520 F05 | EVI1 | 3 | reverse | 170344041 | | | | | | | | |
| 36 | 75918 G04 | EVI1 | 3 | reverse | 170347592 | | | | | | | | |
| 37 | 79207 B11 | EVI1 | 3 | same | 170350543 | T | | | | | | | |
| 38 | 76776 G04 | MDS1 | 3 | reverse | 170351592 | T | | | | | | | |
| 39 | 81520 F05 | MDS1 | 3 | reverse | 170399072 | | | | | | | | |
| 40 | 77049 G11 | MDS1 | 3 | same | 170400813 | | | | | | | | |
| 41 | 76776 E04 | MDS1 | 3 | same | 170411959 | T | | | | | | | |
| 42 | 89252 E08 | MDS1 | 3 | reverse | 170415162 | | | | | | | | |
| 43 | 74718 H10 | MDS1 | 3 | reverse | 170415288 | T | | | | | | | L |
| 44 | 76776 A10 | MDS1 | 3 | reverse | 170433035 | T | | | | | | | |
| 45 | 77509 A03 | MDS1 | 3 | same | 170434026 | | | | | | | | |
| 46 | 76062 D09 | MDS1 | 3 | reverse | 170444844 | | | | | | | L | |
| 47 | 74718 A07 | MDS1 | 3 | reverse | 170451100 | | | | | | L | | |
| 48 | 76062 E05 | MDS1 | 3 | same | 170452341 | | | | | | L | | |
| 49 | 75916 A01 | MDS1 | 3 | same | 170509909 | T, Q | | | | | | Q | Q |
| 50 | 75917 B04 | MDS1 | 3 | reverse | 170516385 | T | | | | | | | |
| 51 | 74718 G05 | MDS1 | 3 | reverse | 170526878 | | | | | | L | | |
| 52 | 76771 D05 | MDS1 | 3 | reverse | 170551923 | T | | | | | | | |
| 53 | 77110 A09 | MDS1 | 3 | reverse | 170553839 | T, Q | | | | | | Q | LTQ |
| 54 | 77049 B02 | MDS1 | 3 | same | 170556473 | | | | | | | | |
| 55 | 76776 A11 | MDS1 | 3 | reverse | 170556716 | T, Q | | | | | | | Q |
| 56 | 75385 B05 | MDS1 | 3 | reverse | 170557515 | | | | | | L | | |
| 57 | 78016 F03 | MDS1 | 3 | reverse | 170557567 | T | | | | | | | |
| 58 | 78016 C11 | MDS1 | 3 | reverse | 170558780 | T | | | | | | | |
| 59 | 75917 H11 | MDS1 | 3 | reverse | 170562183 | | | | | | | | |
| 60 | 75916 A05 | MDS1 | 3 | same | 170563940 | T, Q | | | | | | | |
| 61 | 78372 E08 | MDS1 | 3 | same | 170563955 | | | | | | | | |
| 62 | 77110 F02 | MDS1 | 3 | reverse | 170573011 | | | | | | | | |
| 63 | 77109 E01 | MDS1 | 3 | reverse | 170573083 | | | | | | | | |
| 64 | 76776 G11 | MDS1 | 3 | reverse | 170588924 | T | | | | | | | |
| 65 | 77048 C07 | MDS1 | 3 | same | 170865275 | T | | | | | | | |
| 66 | 75523 E11 | MDS1 | 3 | reverse | 170868261 | | | | | | | | L |
| 67 | 79208 F04 | MDS1 | 3 | reverse | 170868263 | | | | | | | | |
| 68 | 76778 G07 | SETBP1 | 18 | reverse | 40513701 | | | | | | | | |
| 69 | 79274 B06 | SETBP1 | 18 | reverse | 40513716 | | | | | | | | |
| 70 | 77512 B07 | SETBP1 | 18 | reverse | 40513723 | T | | | | | | | |
| 71 | | SETBP1 | 18 | reverse | 40513792 | | | | | | | | |
| 72 | 76778 F12 | SETBP1 | 18 | same | 40513795 | T, Q | | | | | | | Q |
| 73 | 76776 E09 | SETBP1 | 18 | same | 40513912 | T | | | | | | | |
| 74 | 75916 G10 | SETBP1 | 18 | same | 40517135 | T | | | | | | | |
| 75 | 77509 D02 | SETBP1 | 18 | same | 40661930 | T | | | | | | | |

| | | | | | | | | | | 381 | 542 | 542 | 381 | 542 | 542 |
|---|---|---|---|---|---|---|---|---|---|---|---|---|---|---|---|
| CIS# | 157 | 192 | 241 | 269 | 304 | 339 | 381 | 416 | 472 | CD15 | CD15 | CD14 | CD3 | CD3 | CD19 |
| 1 | L | L | | | L | | | | | | | | | | |
| 2 | LTQ | LTQ | LTQ | LTQ | LTQ | LTQ | LTQ | LQ | | TQ | TQ | | | | |
| 3 | L | | | | | | | | | | L | | | | |
| 4 | L | | | | | | | | | | | | | | |
| 5 | L | | | | | | | | | | | | | | |
| 6 | LT | LT | T | | | | | | | | | | | | |
| 7 | L | L | | | | | | T | | | | | | | |
| 8 | T | | L | | | | T | | | | | | | | |
| 9 | | T | T | T | | T | | | | | | | | | |
| 10 | | | | | | | T | | | | | | | | |
| 11 | | T | T | | | | | | | | | | | | |
| 12 | T | | | | | | | | | | | | | | |
| 13 | T | | T | | | | | | | | | | | | |
| 14 | T | | | | | | | | | | | | | | |
| 15 | LT | LT | L | T | | | | | | | | | | | |
| 16 | L | | | | | | | | | | | | | | |
| 17 | | | | | | | | | | | | | | | |
| 18 | T | T | | | | | T | | | | | | | | |
| 19 | L | | L | T | | | | | | | | | | | |
| 20 | L | L | L | T | L | | | | | | | | | | |
| 21 | L | L | L | L | | | | | | | | | | | |
| 22 | L | L | L | | | T | | | | | | | | | |
| 23 | L | L | T | L | | T | | | | | | | | | |
| 24 | L | L | T | L | L | | | | | | | | | | |
| 25 | T | T | T | L | T | | | | | | | | | | |
| 26 | | | L | | | | | | | | | | | | |
| 27 | | | L | | | | | | | | | | | | |
| 28 | | | L | | | | | | | | | | | | |
| 29 | | | L | | | | | | | | | | | | |
| 30 | | L | | L | | | | | | | | | | | |
| 31 | T | T | LT | | | | | | | | | | | | |
| 32 | L | | | | | | | | | | | | | | |
| 33 | L | | | | | | | | | | | | | | |
| 34 | LT | L | T | | | | | | | | | | | | |
| 35 | | | | | | | L | | | | L | L | | | |
| 36 | | L | | | | | | | | | | | | | |
| 37 | | | | | L | | | | | | | | | | |
| 38 | LT | | T | T | | | L | | | | | | T | | |
| 39 | | | | | | | L | | | | | | | | |
| 40 | | | L | | | | L | | | | | | | | |
| 41 | L | | | | | | | | | | | | | | |
| 42 | | L | | | | | | | | | | | | | |
| 43 | | | L | | | | | | | | | | | | |
| 44 | | L | L | T | | T | | | | | | | | | |
| 45 | | | L | L | L | | | | | | | | | | |
| 46 | | | | | | | | | | | | | | | |
| 47 | | | | | | | | | | | | | | | |
| 48 | | | | | | | | | | | | | | | |
| 49 | LTQ | TQ | LTQ | LTQ | LTQ | LTQ | LQ | LTQ | T | TQ | LTQ | LT | T | LT | |
| 50 | L | | L | | | | | | | | | | | | |
| 51 | | | | | | | | | | | | | | | |
| 52 | | | L | L | | | | | | | | | | | |
| 53 | LTQ | LTQ | LTQ | LTQ | LTQ | LTQ | LTQ | LTQ | LTQ | LTQ | LTQ | LTQ | LTQ | Q | LTQ |
| 54 | | | L | L | | | | | | | | | | | |
| 55 | Q | LQ | LTQ | TQ | TQ | Q | Q | T | T | TQ | TQ | | | | |
| 56 | | | | | | | | | | | | | | | |
| 57 | T | T | T | L | | | | LT | | | | | | | |
| 58 | | | T | L | | | | | | | | | | | |
| 59 | L | | L | | | | | | | | | | | | |
| 60 | TQ | LTQ | LTQ | TQ | LTQ | LTQ | LTQ | LTQ | LT | LTQ | TQ | T | T | | |
| 61 | | | | L | | | | | | | | | | | |
| 62 | | | L | | | | | | | | | | | | |
| 63 | | | L | | | | | | | | | | | | |
| 64 | | L | | | | | | | | T | LT | LT | | | T |
| 65 | | L | LT | L | T | L | | L | | | | | | | |
| 66 | | | | | | | | | | | | | | | |
| 67 | | | | | L | | | | | | | | | | |
| 68 | | L | L | | L | | | | | | | | | | |
| 69 | | | | | L | | | | | | | | | | |
| 70 | | | L | | | | | | | | | | | | |
| 71 | T | T | T | T | T | T | T | T | T | T | | | | | |
| 72 | TQ | LTQ | TQ | LTQ | LTQ | TQ | TQ | TQ | T | TQ | TQ | T | T | | T |
| 73 | | L | | | | | | | | | | | | | |
| 74 | LT | | L | | | | | | | | | | | | |
| 75 | | | L | | | | | | | | | | | | |

TABLE 2b

| | | | Chro- | Vector | | | | | | | | | | | | | |
|---|---|---|---|---|---|---|---|---|---|---|---|---|---|---|---|---|---|
| | Sequence | | mo- | Orien- | UCSC | | | | | | | | | | | | |
| CIS# | Identity | Gene | some | tation | Locus | Track | 24 | 28^{§#} | 35^{§#} | 49^{#} | 84 | 119* | 149 | 175 | 245 | 287 | 343 |
| 1 | 78166 C09 | PRDM16 | 1 | reverse | 3011985 | | | | | | | | L | | | | |
| 2 | 78166 B07 | PRDM16 | 1 | same | 3109761 | | | | | | | | L | | L | | |
| 3 | 82774 D06 | PRDM16 | 1 | same | 3109929 | | | | | | | | | | | L | |
| 4 | 78165 H02 | PRDM16 | 1 | reverse | 3111506 | T | | | | | | | LT | LT | L | T | LT |
| 5 | 78165 B07 | PRDM16 | 1 | same | 3113799 | | | | | | | | L | | | | |
| 6 | 78373 B06 | PRDM16 | 1 | reverse | 3121364 | | | | | | | | L | | | | |
| 7 | 81841 E09 | PRDM16 | 1 | reverse | 3121907 | | | | | | | | | | L | | |
| 8 | 78373 G04 | PRDM16 | 1 | reverse | 3123391 | | | | | | | | L | | | | |
| 9 | 79275 E09 | PRDM16 | 1 | same | 3123459 | T | | | | | | | | L | L | | |
| 10 | 78373 F04 | PRDM16 | 1 | same | 3123555 | | | | | | | | LT | | | | |
| 11 | 81673 C08 | PRDM16 | 1 | same | 3123617 | | | | | | | | | T | L | | L |
| 12 | 79275 B07 | PRDM16 | 1 | same | 3123716 | T | | | | | | | | LT | | | |
| 13 | 79272 F07 | PRDM16 | 1 | reverse | 3123809 | T | | | | | | | | L | | | |
| 14 | 79275 D06 | PRDM16 | 1 | same | 3123898 | | | | | | | | | L | | | |
| 15 | 78166 D04 | PRDM16 | 1 | same | 3124033 | T | | | | | | | L | L | L | L | |
| 16 | | PRDM16 | 1 | same | 3124164 | | | | | | T | | | | | | |
| 17 | 78166 H04 | PRDM16 | 1 | same | 3124270 | | | | | | | | L | | | L | |
| 18 | 78373 E04 | PRDM16 | 1 | same | 3124373 | T | | | | | | | L | L | L | | |
| 19 | 78373 H05 | PRDM16 | 1 | same | 3124425 | | | | | | | | L | | | | |
| 20 | 81674 A11 | EVI1 | 3 | same | 170336451 | | | | | | | | | | L | | |
| 21 | 86758 H12 | EVI1 | 3 | same | 170337216 | | | | | | | | | | | | L |
| 22 | 82776 B11 | EVI1 | 3 | same | 170338758 | | | | | | | | | | | L | |
| 23 | 78165 E09 | EVI1 | 3 | same | 170338858 | | | | | | | | L | | | L | |
| 24 | 78166 B03 | EVI1 | 3 | same | 170339841 | T | | | | | | | LT | LT | | | |
| 25 | | EVI1 | 3 | same | 170342633 | | | | | | T | | | | | | |
| 26 | 79275 G07 | EVI1 | 3 | same | 170342651 | T | | | | | T | | | L | | | |
| 27 | 78166 H11 | EVI1 | 3 | same | 170345961 | T | | | | | | | L | | | | |
| 28 | 81673 A07 | EVI1 | 3 | same | 170347808 | | | | | | | | | | L | | |
| 29 | 86611 G04 | EVI1 | 3 | reverse | 170348090 | | | | | | | L | | | | | |
| 30 | 88283 H11 | MDS1 | 3 | same | 170348423 | | | | | | | | | | | | L |
| 31 | 85439 A02 | MDS1 | 3 | reverse | 170350896 | | | | | | | | | | L | | |
| 32 | 81673 H07 | MDS1 | 3 | reverse | 170352049 | | | | | | | | | | L | | |
| 33 | 87429 F02 | MDS1 | 3 | same | 170355075 | | | | | | | | | | | | L |
| 34 | 81673 D07 | MDS1 | 3 | same | 170366741 | | | | | | | | | | L | L | |
| 35 | 81673 F06 | MDS1 | 3 | reverse | 170396907 | | | | | | | | | | L | | |
| 36 | 81676 B02 | MDS1 | 3 | same | 170415074 | | | | | | | | | | L | | |
| 37 | 87429 A02 | MDS1 | 3 | reverse | 170415363 | | | | | | | | | | L | | L |
| 38 | 81674 B09 | MDS1 | 3 | reverse | 170444820 | | | | | | | | | | L | | |
| 39 | 82776 E03 | MDS1 | 3 | reverse | 170445204 | | | | | | | | | | | L | L |
| 40 | 78166 E03 | MDS1 | 3 | same | 170449882 | T | | | | | | | L | | | | |
| 41 | 82774 G01 | MDS1 | 3 | same | 170450331 | | | | | | | | | | | L | |
| 42 | 81674 A12 | MDS1 | 3 | same | 170508606 | | | | | | | | | | L | L | L |
| 43 | 81674 D05 | MDS1 | 3 | reverse | 170534821 | | | | | | | | | | L | L | L |
| 44 | 81676 C08 | MDS1 | 3 | same | 170536132 | | | | | | | | | | L | | |
| 45 | 78166 D08 | MDS1 | 3 | reverse | 170536218 | T | | | | | | | L | | | | |
| 46 | 81676 B08 | MDS1 | 3 | same | 170545797 | | | | | | | | | | L | | |
| 47 | 81675 E02 | MDS1 | 3 | reverse | 170546186 | | | | | | | | | | L | L | L |
| 48 | 81674 G07 | MDS1 | 3 | same | 170548107 | | | | | | | | | | L | | L |
| 49 | 78165 D10 | MDS1 | 3 | same | 170552880 | T, Q | | | | | | Q | LQ | TQ | LTQ | LTQ | LT |
| 50 | 81674 A02 | MDS1 | 3 | reverse | 170553197 | | | | | | | | | | L | | |
| 51 | 82774 B05 | MDS1 | 3 | reverse | 170554755 | | | | | | | | | | | L | |
| 52 | 86612 A01 | MDS1 | 3 | same | 170555336 | | | | | | | | | | | L | |
| 53 | 78166 B04 | MDS1 | 3 | same | 170555455 | T, Q | | | | | | Q | LTQ | LTQ | LQ | LTQ | LT |
| 54 | 87429 A09 | MDS1 | 3 | same | 170555532 | | | | | | | | | | | | L |
| 55 | 81674 A05 | MDS1 | 3 | same | 170555633 | | | | | | | | | | L | | |
| 56 | 82774 G04 | MDS1 | 3 | reverse | 170556130 | | | | | | | | | | | L | |
| 57 | 81674 G12 | MDS1 | 3 | reverse | 170556199 | | | | | | | | | | L | L | |
| 58 | 86758 B06 | MDS1 | 3 | reverse | 170556399 | | | | | | | | | | | | L |
| 59 | 78165 B06 | MDS1 | 3 | reverse | 170557382 | | | | | | | | L | | | | |
| 60 | 81676 A05 | MDS1 | 3 | reverse | 170557818 | | | | | | | | | | L | L | |
| 61 | 78166 G05 | MDS1 | 3 | reverse | 170559264 | | | | | | | | L | | | L | |
| 62 | 82774 C01 | MDS1 | 3 | same | 170562559 | | | | | | | | | | | L | L |
| 63 | 81676 A06 | MDS1 | 3 | reverse | 170568247 | | | | | | | | | | L | | |
| 64 | 79275 E08 | MDS1 | 3 | reverse | 170588540 | T, Q | | | | | | Q | Q | LTQ | LQ | LTQ | LT |
| 65 | 81840 E12 | MDS1 | 3 | reverse | 170588629 | | | | | | | | | | L | | |
| 66 | 81841 E06 | MDS1 | 3 | reverse | 170588996 | | | | | | | | | | L | | L |
| 67 | 78166 H03 | MDS1 | 3 | same | 170722319 | T | | | | | | | LT | | L | L | |
| 68 | 81674 D06 | MDS1 | 3 | reverse | 170865957 | | | | | | | | | | L | | |
| 69 | 77510 A09 | MDS1 | 3 | same | 170906546 | | | | | | L | | | | | | |

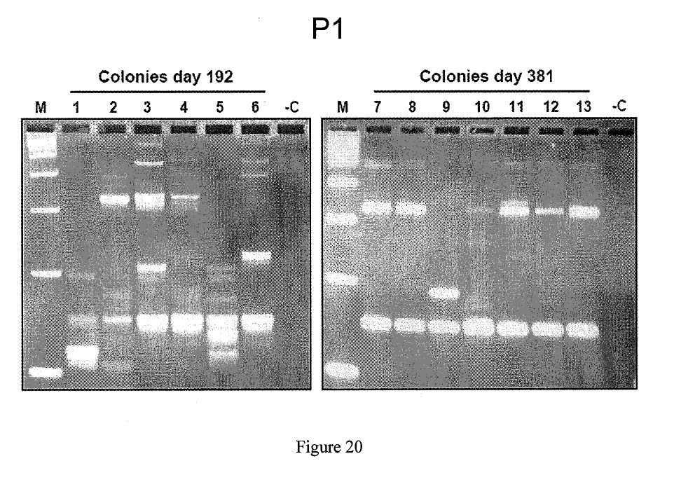

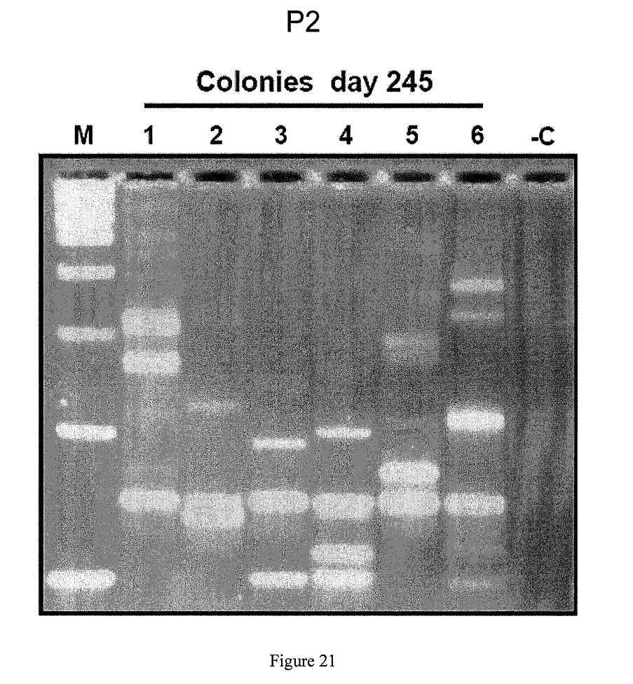

Quantitative-competitive PCR was then used to further analyze the dominant clones (as described in Example 5). A spiked internal standard was used to test for clinically relevant continued proliferation. Stable activity was observed for a period of between 5 to 14 months (FIGS. 14, 15, 18, and 19). The most productive clone in patient P1 contained two insertions, one in intron 2 of the MDS1 gene locus and the other one in an intergenic DNA region. This clone's quantitative contribution to the transduced cell pool was first detected by LAM-PCR at +122 days post transplant. From day +122 on, it then increased until it peaked at about 80% of gene-modified cells present in the peripheral blood at day +381. So far, it has remained at this level until the last time point analyzed (day +542). Detection of this clone was also conducted by QC-PCR in sorted granulocytes, B and T cells at day +542 indicating the multilineage potential of the initial transduced cells (Table 2). The increasing dominance of this clone was also documented by integration site analysis and locus specific PCR of bone marrow progenitors (CFU-GM and BFU-E). Although at day +192 only 3 out of 6 (3 out of 11 by locus specific PCR) vector-containing colonies contained the same two insertion bands, the dominant clone contributed to 6 out of 7 (28 out of 36 by locus specific PCR) colonies at day +381 (FIG. 20). Analysis of five additional clones revealed shared integration sites between CD3+ cells, CD19+ cells and CD15+ cells obtained from P1 at days +381 and +542, again suggesting effective gene transduction of hematopoietic stem cells (HSC) (FIGS. 9, 18, and Table 2). In P2, no single clone had a strong dominance, up to day +343 (FIG. 21). Approximately 1.5 to 2.6 insertions are thought to be present in the gene modified cell transplants based on the average copy number per CD34 cell transplanted and its relation to the percentage of gp91^{phox} protein expression in CD34 cells infused. In line with this average, LAM-PCR analysis of colonies sampled from long-term repopulating cells demonstrated that the CFU colonies contained between 1 and 4 integrants per cell (FIGS. 20, 21).

The highest frequency of PRDM16 related integration sites retrieved from patient P1 by LAM-PCR was obtained at day +157 (30% of the transduced cell pool) and then continuously decreased until day +542 (1.1%). In patient P2, the frequency of PRDM16 inserted clones decreased from day +175 (23.7%) to day +343 (12.8%). Conversely, during the same time period, the frequency of MDS1/EVI-1 integrants increased in P1 from 12% to 90.1% and in P2 from 20.6% to 64.9%. On day +304, SETBP1 insertions accounted for 8.4% of all integrants in P1, but from day +339 no further SETBP1 insertions were detected by LAM-PCR. Residual activity of individual SETBP1 clones could be detected by tracking PCR on days +381, +416, +472 and +542 (Table 2).

The mechanistic relevance of these insertions can be demonstrated by the detection of specific mRNA transcripts in bone marrow (BM) from P1. Elevated levels (>1 log) of PR domain positive MDS1/EVI-1, PRDM16 and of SETBP1 mRNA transcripts were found by RT-PCR.

As demonstrated herein, retrovirus gene activation can occur as a consequence of any retrovirus vector insertion event, and may be of influence on the biological fate of the target cell. The location of an insertion defines the likelihood of whether such events lead to side effects, ultimately depending on the biological relevance of a gene for the affected cell type, in this case hematopoiesis. This data is of very significant influence for the efficacy and biosafety assessment of gene therapy vectors in ongoing and future clinical trials. Depending on the clinical outcome, this insertional side effect, very likely favored by reinfusion of high numbers of gene corrected CD34+ BM cells containing insertion events, may have facilitated the therapeutic success observed.

The above described analysis demonstrates a previously unknown role of PR domain genes and SETBP1 in the proliferation of morphologically normal long-term repopulating progenitor cells. This finding can be used to treat a number of mammalian diseases, as described below.

### Functional Properties of MDS1/EVI-1, PRDM16, and SETBP1 Clones

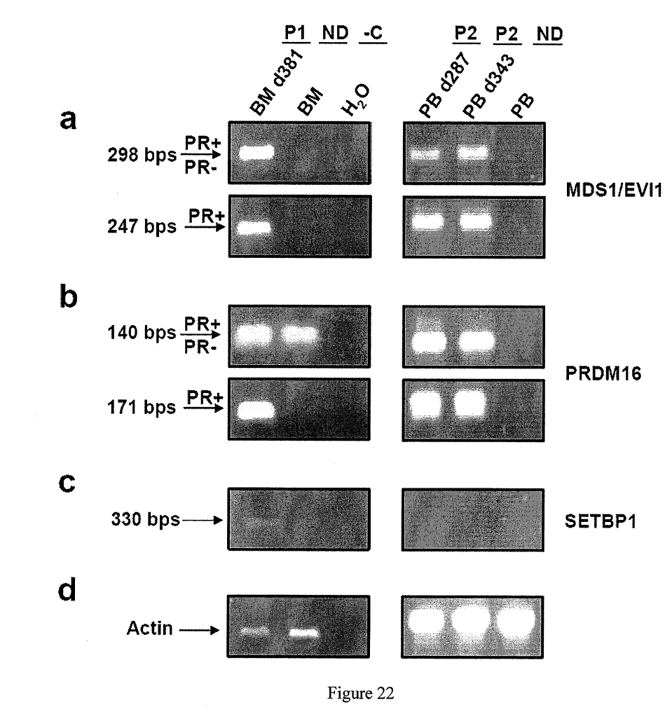

To confirm the functional influence of these insertions via gene activation, specific mRNA transcripts were analyzed by RT-PCR (as described in Example 6). At day +381 bone marrow cells from patient P1 contained substantially elevated levels of both MDS1/EVI-1 and of SETBP1 mRNA transcripts, whereas PRDM16 transcripts were present at levels comparable to control bone marrow (FIG. 22). RNA microarray analysis of the same sample using the Affymetrix HG U133_Plus_{—}2.0 Array confirmed overexpression of MDS1/EVI-1 or EVI-1 (36-fold) and SETBP1 (32-fold). Abnormal expression of PRDM16 was not found. RT-PCR performed on RNA samples obtained from peripheral blood leukocytes from patient P2 at days +287 and +343 showed overexpression of MDS1/EVI-1 and PRDM16, while SETBP1 transcripts were not detected. A microarray analysis of the same samples revealed a 74-fold overexpression of MDS1/EVI-1.

Transduced cells were strictly dependent on growth factors for proliferation and differentiation. No colony formation was observed when bone marrow mononuclear cells (patient P1: days +122, +192 and +241) were plated on methylcellulose and cultured for 14 days in the absence of cytokines (as described in Example 7). Colony forming cells (CFCs) derived from CD34+ cells of patient P1 at day +381 were replated in the presence of cytokines into secondary and tertiary methylcellulose cultures. Few cell clusters were visible after the second replating, while no growth was observed in further replatings, indicating the absence of self-renewal capacity. Similar results were obtained with cells from patient P2 at day +245. Furthermore, 1000 human CD34+ cells derived from patient P1 at day +381 were injected into each of two nude nonobese diabetic-severe combined immunodeficient (NOD-SCID) B2m^{-/-} mice. No engraftment of CD45+ cells in these mice were observed.

### Functional Reconstitution of Phagocytic Killing Activity

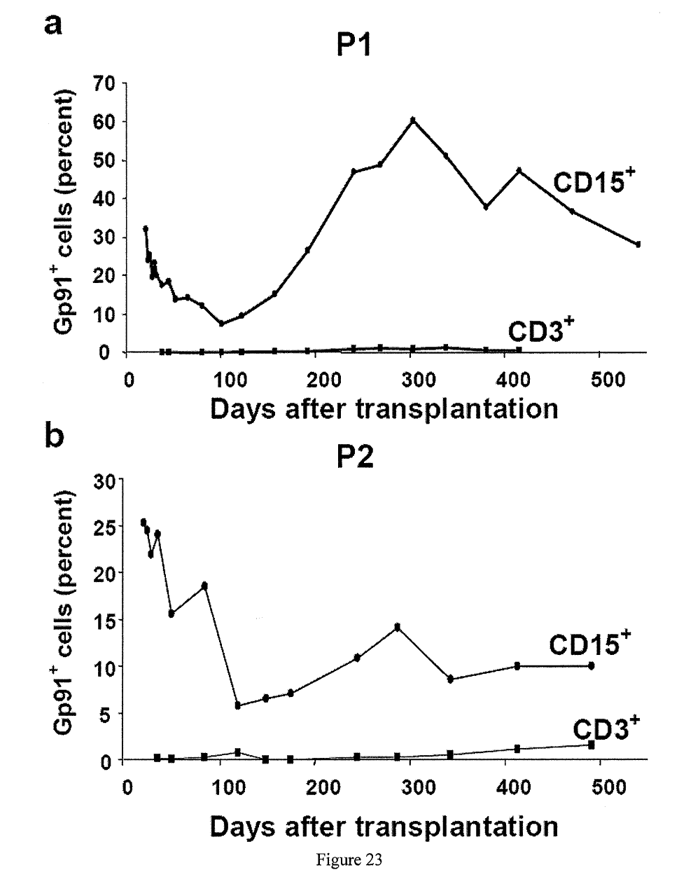

Expression of gp91^{phox} was detected by FACS using the monoclonal antibody 7D5 (as described in Example 8 and Yamauchi, A. et al. Location of the epitope for 7D5, a monoclonal antibody raised against human flavocytochrome b558, to the extracellular peptide portion of primate gp91^{phox}. Microbiol Immunol 45, 249-257 (2001), herein incorporated by reference in its entirety). Gp91^{phox} was present mainly in CD15+ cells with as many as 60% (patient P1, day +304) and 14% (patient P2, day +287) of the cells expressing the transgene. Correctly assembled flavocytochrome_b558 heterodimers were found by spectroscopy in cell membrane extracts from granulocytes obtained from P1 and P2. Gp91^{phox} expression was also detected in bone marrow derived CD34+ cells from P1 +381 days post-transplantation (FIGS. 23,24).

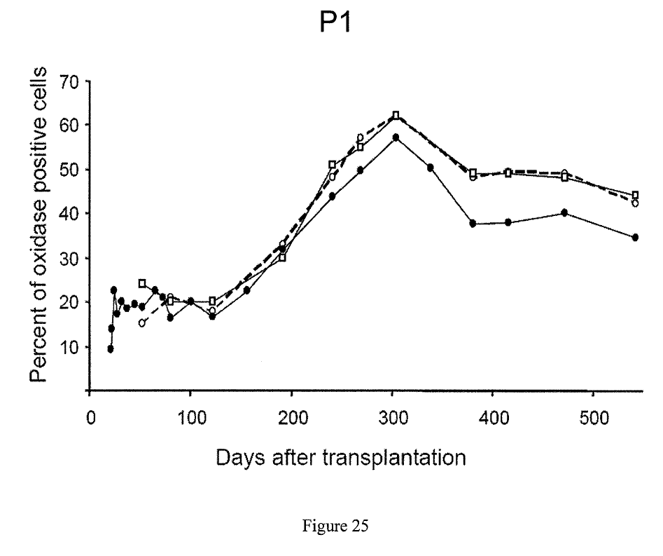

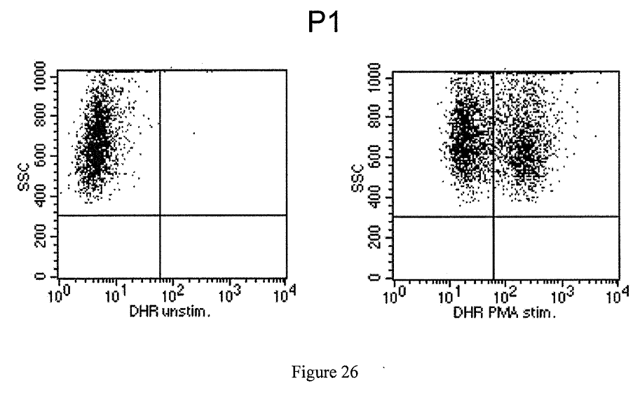

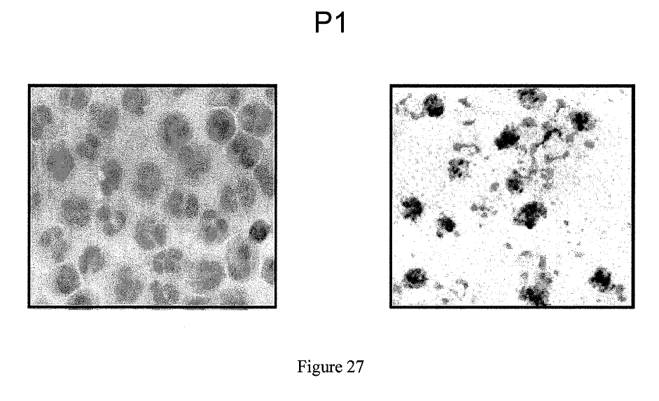

Functional reconstitution of respiratory burst activity in peripheral blood leukocytes (PBLs) was assayed after stimulation with opsonized E. coli by the dihydrorhodamine (DHR) 123 assay (FIGS. 25,28) (as described in Example 12). NADPH oxidase activity was detected in 10% to 20% of P1 leukocytes until day +122. Thereafter, a strong increase in the number of oxidase positive cells was observed. As many as 57% of patient P1's leukocytes tested positive for superoxide production at day +304, followed by a decrease to 34.4% at day +542 (FIG. 25). Similar results were obtained with purified granulocytes after stimulation with phorbol 12-myristate 13-acetate (FIG. 26) or by monitoring the reduction of nitroblue tetrazolium (NBT) to formazan in gene corrected neutrophils (FIG. 27).

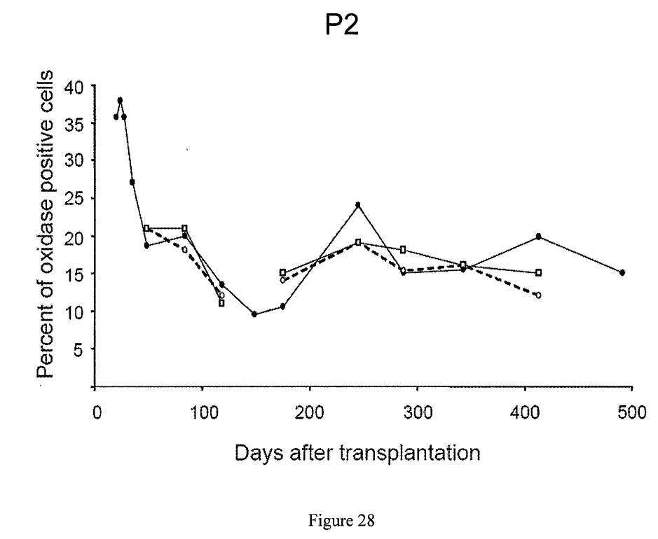

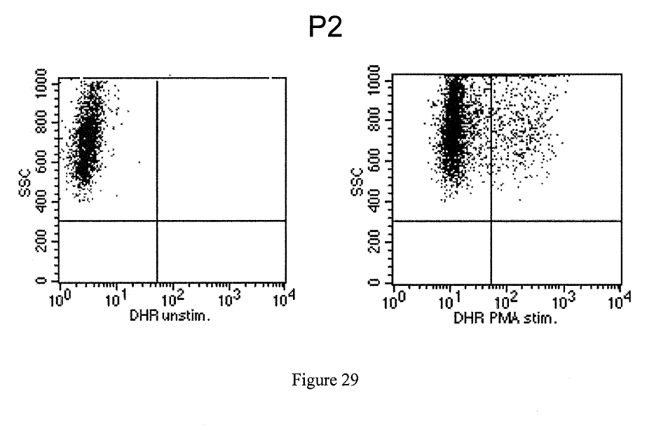

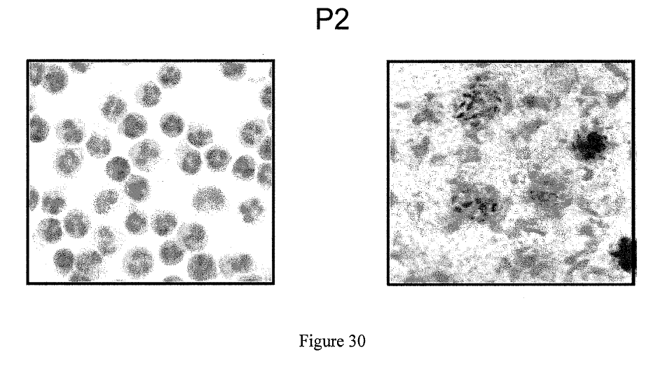

The time course of superoxide production was very similar in patient P2. The number of oxidase positive cells was high (>35%) shortly after infusion of gene-transduced cells, but decreased to 9.6% at day +149 post-transplantation. Subsequently, an increase in the number of oxidase positive cells of up to 24% (day +245) was observed (FIGS. 28, 29). This value decreased to 15.3% at day +287 and fluctuated thereafter between 19.8% (day +413) and 15% (day +491). These results were confirmed by the NBT assay on individual neutrophils (FIG. 30).

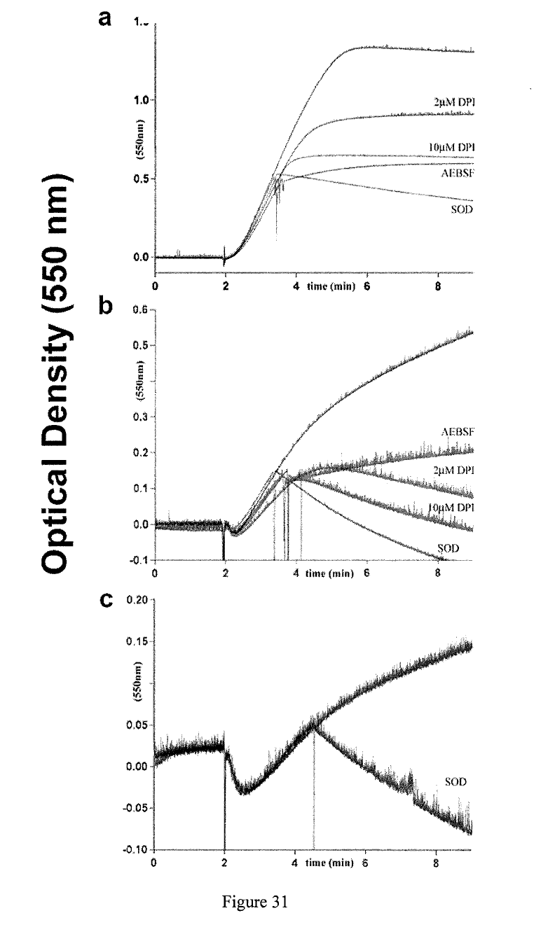

Superoxide production was quantified in patient neutrophils by the cytochrome C reduction assay [Mayo, L. A. & Curnutte, J. T. Kinetic microplate assay for superoxide production by neutrophils and other phagocytic cells. Methods Enzymol 186, 567-575 (1990), herein incorporated by reference in its entirety]. Total neutrophils obtained from patient P1 at day +193 produced 1.23 mmol superoxide/10⁶ cells/min, which corresponds to 4.13 nmol/10⁶ cells/min after correction for the number of oxidase positive cells at this time point (33%). Similarly, total neutrophils from patient P2 at day +50 produced 2.12 nmol superoxide/10⁶ gene-corrected cells/min. In comparison, the amount of superoxide produced by wild type neutrophils was 14.35±6.28 mmol superoxide/10⁶ cells/min (n=10; FIG. 31).

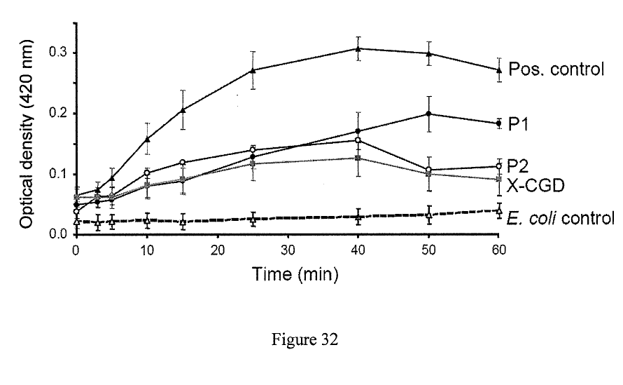

Since the level of superoxide production in gene-corrected cells was at most one-third to one-seventh of the level measured in wild type cells, these cells were tested to determine whether they could kill ingested microorganisms. Bacterial killing was measured by monitoring β-galactosidase activity released by engulfed and perforated E. coli (as described in Example 9 and by Hamers, M. N., Bot, A. A., Weening, R. S., Sips, H. J. & Roos, D. Kinetics and mechanism of the bactericidal action of human neutrophils against Escherichia coli. Blood 64, 635-641 (1984), herein incorporated by reference in its entirety). In this assay, X-CGD cells showed minimal β-Gal activity due to impaired perforation capacity in the absence of superoxide production (FIG. 32). In contrast, gene corrected granulocytes obtained from patients P1 (day +473) and P2 (day +344) showed a substantial increase in β-Gal activity, illustrating improvement in antibacterial activity in neutrophils of both patients after gene therapy.

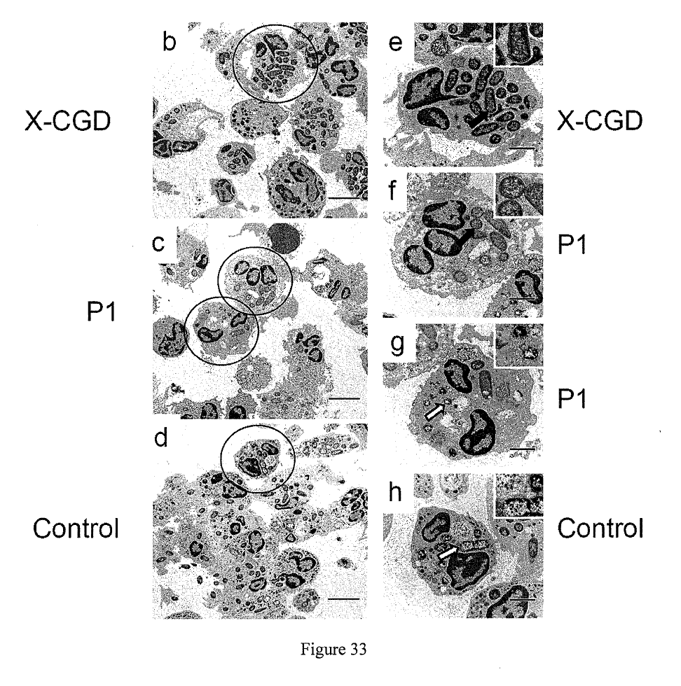

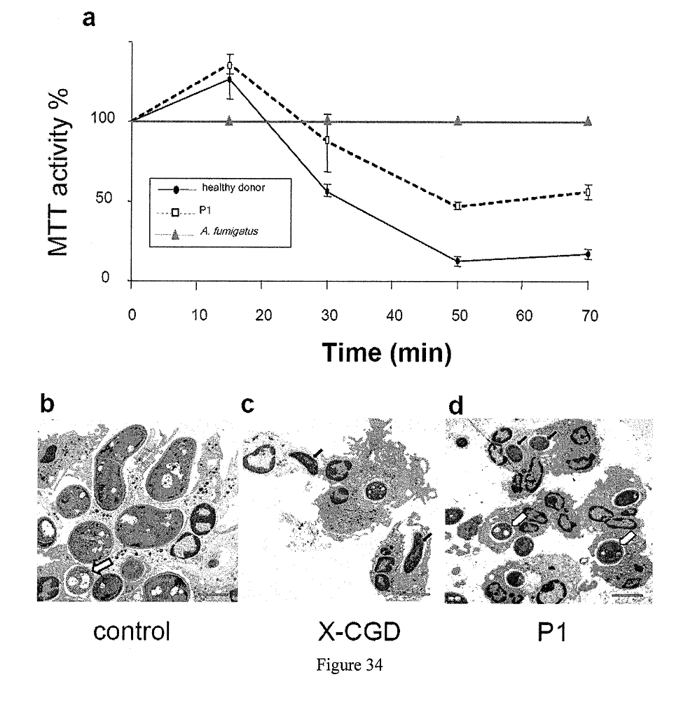

These results were confirmed by electron microscopy visualization of bacterial killing by healthy, X-CGD or gene corrected neutrophils from patient P1 (as described in Example 10 and illustrated in FIG. 33). Phagocytosis of E. coli was observed in all samples. However, the morphology of E. coli inside of the phagocytic vacuole differed drastically between specimens. While the vast majority of E. coli ingested by X-CGD granulocytes were not degraded (FIGS. 33b,e), E. coli ingested by wild type granulocytes showed clear signs of degradation as revealed by necrotic microorganisms with irregular morphology (FIGS. 33 d,h). Neutrophils from patient P1 consisted of a mixture of cells with clear bacterial degradation (lower circle, FIGS. 33c,g), and others without signs of bacterial degradation that were indistinguishable from non-corrected controls (upper circle, FIGS. 33c,f). Similarly, gene corrected granulocytes obtained from P1 at day +381 were able to degrade Aspergillus fumigatus hyphae as demonstrated by an enzymatic assay [Rex, J. H., Bennett, J. E., Gallin, J. I., Malech, H. L. & Melnick, D. A. Normal and deficient neutrophils can cooperate to damage Aspergillus fumigatus hyphae. J Infect Dis 162, 523-528 (1990), herein incorporated by reference in its entirety] and transmission electron microscopy (FIG. 34).

### Clinical Resolution of Infection

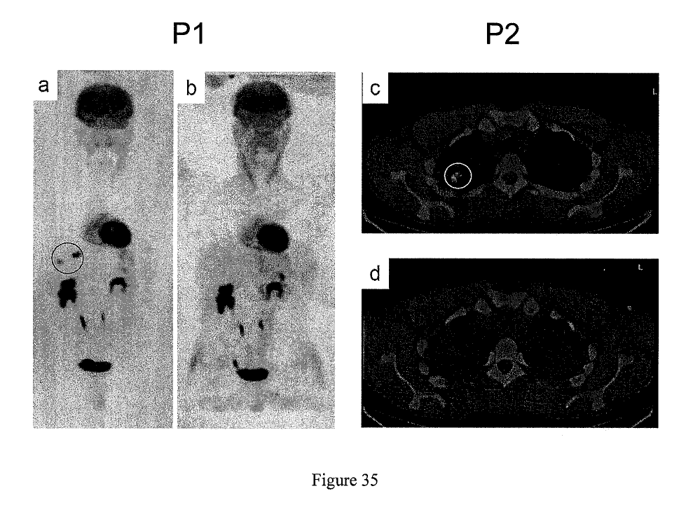

Prior to gene therapy, the combination of whole body positron emission tomography (PET) and computed tomography (CT) scanning (as described in Example 13) revealed an active bacterial or fungal infection in each of the two patients. For patient P1, a high focal uptake of fluorine-18-fluoro-2-deoxy-D-glucose (¹⁸F-FDG) was observed in two hypodense lesions in liver segments VII/VIII and VIII, representing Staphylococcus aureus abscesses (FIG. 35a, circle). Similarly, patient P2 had suffered from severe invasive pulmonary aspergillosis due to A. fumigatus, visualized by ¹⁸F-FDG uptake in PET/CT scanning as a cavernous cavity extending from the apical to the posterior segment of the superior lobe on the right side (FIG. 35c, circle). Repeat scans performed 50 days after administration of gene-transduced cells showed no evidence of lesions in the liver of patient P1 (FIG. 35b), while only minimal ¹⁸F-FDG activity was evident at day +53 post therapy in the cavity wall of patient P2 (FIG. 35d). Follow-up analysis of the patients has not revealed any reappearance of these lesions. These and other clinical parameters (as described in Examples 14-20) demonstrate that gene therapy provided a therapeutic benefit to both patients.

### Treatment to Increase Cell Proliferation by Administering a Retroviral Vector

In some embodiments of the invention, a patient in need of hematopoietic cell proliferation can be treated by retroviral insertion methods. For example, a patient cell sample can be transfected with a retroviral or other type of gene vector carrying these genes, or activating their cellular alleles, using methods known to those of skill in the art. The cells can then be reinfused into the patient. Cell counts can be performed periodically to determine the effectiveness of the blood cell proliferation treatment. The amount of cells to be transfected, the ratio of viral vector to cells, cell growth methods, and readministration methods can be varied as needed to treat the particular disorder. The progress can be followed, for example, by LAM-PCR to confirm the activation of EVI-related genes. (FIGS. 9-10, 14-15)

If desired, the retroviral vector or other gene vector can be administered to the patient directly, rather than to cells that have been isolated from the patient.

The method can be used to expand any type of mammalian cell. Examples of the types of cell that may be expanded include but are not limited to a stem cell, an embryonic stem cell, an adult stem cell, a multipotent stem cell, a pluripotent stem cell, a hematopoietic cell, a hematopoietic stem cell, a progenitor cell, a myelopoietic stem cell, a peripheral blood cell, non-hematopoietic stem cells or progenitor cells, and the like. The cells to be treated can be present in a cell culture, or can be present in the body.

The retroviral vector insertion or other vector transfer can be used to treat many cell-based diseases, in addition to the CGD shown herein. Any disease where an increase in cell proliferation is helpful can be treated by the method of the invention. Examples of such diseases include but are not limited to inherited diseases (severe combined immunodeficiencies, anemias like Fanconi anemia), cancer, AIDS, and the like.

The invention can, in some embodiments, be used to predict the insertion location of a retroviral vector insertion in one patient, by following previous insertion results of another patient or similar animal and in vivo models. For example, in the current CGD analysis, the earlier studied successfully treated patient had activating DNA insertions in similar positions as those of the later studied successful patient. This can be especially useful for early prediction of the likelihood of successful treatment. Further, a knowledge of where a successful insertion is likely to be located can make more simple assays, such as dipstick assays for EVI-1 (or related gene) gene or protein expression useful for a quick test to see if a patient is responding to treatment.

In some embodiments of the invention, a patient in need of gene-correction can be treated. The gene correction can be performed in an in vitro culture of cells isolated from the patient. To increase the proliferation of the gene-corrected cells, activation of the EVI-related genes can be performed. This can be done, for example, by administering a retroviral vector to the culture of gene corrected cells, allowing the cells to proliferate in vitro, then reinfusing or readministering said cells to the patient. These retroviral treated cells are then both gene corrected and fast growing, allowing the patient to receive the gene therapy more rapidly. Many types of gene corrections can be performed using this method. Examples of suitable genes for correction include but are not limited to single gene or multiple gene inherited disorders of the blood forming and immune system or other body tissues that can be complemented, treated or stabilized by gene transfer, and the like. Examples include but are not limited to X-SCID, ADA-SCID, CGD, alpha 1 antitrypsin deficiency, and the like.

Methods of Treatment Involving Transfection of Cells with EVI-Related Genes and SETBP1

Because of the surprising finding that the repopulated cells of the successfully treated CGD patients had activating insertions in the EVI-related genes and SETBP1, it is likely that other methods of increasing levels of EVI-related and SETBP1-related gene products can increase proliferation rates. Accordingly, in some embodiments of the invention, a nucleic acid encoding EVI-1, PRDM16, or SETBP1 is operably linked to a transcriptional regulatory sequence, and transfected to a cell. The exogenous nucleic acid can be, for example, integrated into the genome, or can be present in the cell, for example, in the cytoplasm on a cytoplasmic vector. Thus, the nucleic acid can be stably or transiently expressed, transferred in synthetic form, including nucleic acid equivalents or mRNAs. The transcriptional regulator sequence, such as a promoter, can be chosen, for example, so as to allow for constitutive expression, conditional expression, or inducible expression.

Further, EVI-1, PRDM16, or SETBP1 polypeptides, or fragments thereof, can be administered to a cell. In some embodiments, active synthetic peptide analogs derived from EVI-1, PRDM16, or SETBP1 polypeptide sequences can be administered to a cell, either in culture or in a patient, to allow increased cell proliferation.

It may be desirable to grow cells that express the EVI-related and/or SETBP1 genes for a short period of time only, in order to increase the rate of cell proliferation. This can be achieved, for example, using specific inducible promoters or transient expression methods as known in the art. In such situations, when the high rate of cell proliferation is achieved, the expression of the EVI-related and SETBP1 genes can be turned off by, for example, removing the inducing agent from the cell environment.

The method can be suitable for increasing the proliferation of cells that are gene-corrected, or non-corrected. The method can be used for increasing the proliferation of any type of mammalian cell.

Depending on the desired effect, EVI-related and SETBP1-related gene expressing cells can be allowed to proliferate for several cycles before being reinfused into the patient. For example, the cells can proliferate for about 1, 3, 5, 8, 10, 13, 17, or 20 or division cycles, prior to reinfusion into the patient, if desired.

Agents that Upregulate EVI-Related and SETBP1 Genes

In additional embodiments of the invention, cell proliferation can be increased, either in vitro or in vivo, by contacting the cells to be proliferated with agents that can upregulate or modulate endogenous EVI-related and SETBP1 genes. Cell culture assays can be performed to determine candidate agents from a library of potential compounds, if desired. Test compounds that modulate EVI-related and SETBP1 gene expression are then chosen for further testing. This method can be used to find pharmaceutically valuable agents that can increase cell proliferation in vitro or in vivo.

### Expansion of Gene-Corrected Cells

Many gene therapy methods involve obtaining a cell from a patient in need of gene correction, then transforming the cell to add a corrected copy of a gene. The cell is then proliferated and eventually the patient is readministered with a large amount of corrected cells. One common problem with such a gene-corrected cells may grow slowly, and may not be able to repopulate the patient adequately for a noticeable improvement to occur.

In such situations, the addition of an EVI-related gene as described herein, such as EVI-1, PRDM16, or SETBP1, and the like, either constitutively or transiently, can increase the proliferation of the gene-corrected cells so that successful readministration and treatment is more likely to occur. This modulation of EVI-related gene expression can be done by several means, such as simply administering the retroviral vector to gene-corrected cells, or, for example, by traditional molecular cloning methods.

In some embodiments of the invention, a method of forming a bodily tissue is provided, by obtaining a desired cell type from a patient, if desired, treating the cell with a nucleic acid to accomplish a gene-correction, treating the cell to allow for increased expression of an EVI-related and/or SETBP1 gene to cause increased cell proliferation, and treating the cell so as to form a desired tissue. The tissue can then be readministered into the patient as a form of gene therapy.

Use of LAM-PCR to Identify Genes that Increase Cell Proliferation Using LAM-PCR

Additional embodiments of the invention provide for a method of identifying genes whose modulation (such as upregulation or downregulation) can increase the proliferation rate, selective advantage, or persistence of a stem or progenitor cell. The method can involve obtaining a transfected cell, allowing it to proliferate for several cycles, then testing using LAM-PCR to determine where the successfully repopulated cells have the nucleic acid insertions. The testing can be performed, if desired, over a period of time to determine how the insertion sites change over time. Candidate genes can then be chosen for further analysis. As an example of this method, Table 1 shows a list of exemplary genes found to contain retroviral insertions in at least one of the two successfully treated CGD patients.

Insertion Sites for Nucleic Acid Insertion that Allow for Increasing Cell Proliferation

As shown herein, integration of an exogenous sequence into specific regions of the genome resulted in an increase in cell proliferation, selective advantage, or persistence. A representative example of such integration site sequences (50 bp genomic DNA in bold and 50 bp vector DNA underlined) is shown below:

5′ CTTCTCTGGAAAATTCCTCATAAGAAAACTGAAATTCAAGCTCCTGC
TCGTGAAAGACCCCACCTGTAGGTTTGGCAAGCTAGCTGCAGTAACGCCA
TTT 3′

Many other insertion sites, as well as genes, identified to be downstream of these insertion sites, are shown in Table 1. These genes include but are not limited to MGC10731, PADI4, CDA, CDW52, ZBTB8, AK2, FLJ32112, TACSTD2, FLJ13150, MGC24133, NOTCH2, NOHMA, EST1B, PBX1, PLA2G4A, HRPT2, ATP6V1G3, PTPRC, NUCKS, CABC1, LOC339789, PRKCE, AFTIPHILIN, NAGK, MARCH7, DHRS9, PRKRA, SESTD1, MGC42174, CMKOR1, TBC1D5, THRB, MAP4, IFRD2, ARHGEF3, FOXP1, ZBTB20, EAF2, MGLL, PLXND1, SLC9A9, SELT, CCNL1, MDS1, BCL6, MIST, STIM2, TEC, OCIAD1, FLJ10808, SEPT11, PRKG2, MLLT2, PGDS, MANBA, SRY1, SET7, MAML3, DCTD, CARF, IRF2, AHRR, POLS, ROPN1L, FLJ10246, IPO11, C2GNT3, SSBP2, EDIL3, SIAT8D, FLJ20125, GNB2L1, C6orf105, JARID2, C6orf32, HCG9, MGC57858, TBCC, SENP6, BACH2, REPS1, HDAC9, OSBPL3, HOXA7, CALN1, FKBP6, NCF1, HIP1, GNAI7, ZKSCAN1, MGC50844, LOC346673, CHRM2, ZH3HAV1, REPIN1, SMARCD3, CTSB, ADAM28, LYN, YTHDF3, SMARCA2, C9orf93, NPR2, BTEB1, ALDH1A1, AUH, C9orf3, WDR31, CEP1, GSN, RABGAP1, ZNF79, CUGBP2, C10orf7, PTPLA, PLXD2, ACBD5, PRKG1, MYST4, IFIT1, C10orf129, CUEDC2, FAM45A, GRK5, OR52NI, OR2AG2, ZNF143, C11orf8, LMO2, NGL-1, DGKZ, NR1H3, KBTBD4, C1QTNF4, MGC5395, ARRB1, FLJ23441, FGIF, MAML2, LOC196264, HSPC063, ELKS, CACNA2D4, CHD4, EPS8, LRMP, NEUROD4, RNF41, FAM19A2, RASSF3, PAMC1, PLXNC1, DAP13, MGC4170, FLJ40142, JIK, CDK2AP1, GPR133, PCDH9, C13orf25, ABHD4, AP4S1, MIA2, RPS29, PSMC6, RTN1, MED6, C14orf43, C14orf118, RPS6KA5, GNG2, PAK6, B2M, ATP8B4, TRIP4, CSK, MESDC1, RKHD3, AKAP13, DET1, DKFZp547K1113, SV2B, LRRK1, CHSY1, TRAF7, ZNF205, ABCC1, THUMPD1, IL21R, MGC2474, N4BP1, SLIC1, CDH9, GPR56, ATBF1, ZNRF1, CMIP, MGC22001, C17orf31, SAT2, ADORA2B, TRPV2, NF1, LOC117584, MLLT6, STAT5A, STAT3, HOXB3, HLF, MAP3K3, SCN4A, ABCA10, EPB41L3, ZNF521, RNF125, SETBP1, FLJ20071, CDH7, MBP, MBP, NFATC1, GAMT, MOBKL2A, NFIC, CALR, GPSN2, ZNF382, EGLN2, PNKP, LAIR1, ZNF579, SOX12, C20orf30, PLCB1, SNX5, LOC200261, ZNF336, BAK1, SPAG4L, EPB411L1, NCOA3, KIAA1404, STIMN3, CBR3, DYRK1A, CSTB, C22orf14, UPB1, MN1, XBP1, C22orf19, RBM9, MYH9, TXN2, PSCD4, UNC84B, FLJ2544, ZCCHC5, MST4, IDS, UTY, SKI, PRDM16, PARK7, CHC1, ZMYM1, INPP5B, GLIS1, SLC27A3, ASH1L, SLAMF1, PBX1, CGI-49, ELYS, RNF144, FAM49A, FLJ21069, SFRS7, SPTBN1, TMEM17, ARHGAP25, FLJ20558, CAPG, PTPN18, RBMS1, LOC91526, KLF7, FLJ23861, CMKOR1, CRBN, ITPR1, RAFTLIN, TNA, CCDC12, FHIT, VGL-3, PPM1L, EVI-1, MDS1, HDSH3TC1, DHX15, TMEM33, CXCL3, EPGN, LRBA, FLJ25371, CPE, POLS, PTGER4, LHFPL2, C5orf12, CETN3, PHF15, PFDN1, KIAA0555, GNB2L1, HLA-E, SLC17A5, UBE2J1, BACH2, HIVEP2, SNX8, TRIAD3, RAC1, ARL4A, ELMO1, BLVRA, SUNC1, ABCA13, GTF2IRD1, RSBN1L, ADAM22, MLL5, IMMP2L, SEC8L1, FLJ12571, CUL1, ANGPT1, DEPDC6, EPPK1, MLANA, MLLT3, SMU1, TLE4, C9orf3, ABCA1, STOM, RABGAP1, NEK6, NR5A1, MGC20262, FLJ20433, MAP3K8, ARHGAP22, C10orf72, TACR2, NKX2, OBFC1, VTI1A, ABLIM1, FLJ14213, MS4A3, B3GNT6, NADSYN1, CENTD2, MAML2, ATP5L, FLI1, CACNA1C, HEBP1, MLSTD1, IPO8, ARID2, SLC38A1, KRT7, USP15, KIAA1040, WIF1, CGI-119, DUSP6, FLJ11259, CMKLR1, SSH1, TPCN1, FLJ42957, JIK, FLT3, TPT1, FNDC3, ARHGAP5, ARF6, GPHN, C14orf4, STN2, PPP2R5C, CDC42BPB, CEP152, OAZ2, AKAP13, CHSY1, CRAMP1L, MHC2TA, NPIP, SPN, MMP2, DKFZp4341099, SIAT4B, PLCG2, MYO1C, C17orf31, MGC51025, WSB1, TRAF4, SSH2, HCA66, RFFL, DUSP14, TCF2, ZNF652, STXBP4, HLF, MSI2, VMP1, HELZ, TREM5, RAB37, SEC14L1, SEPT9, BIRC5, PSCD1, MGC4368, NDUFV2, C18orf25, ATP8B1, CDH7, FLJ44881, NFATC1, C19orf35, GNG7, MATK, C3, ZNF358, LYL1, F2RL3, ZNF253, ZNF429, KIAA1533, U2AF1L3, GMFG, BC-2, C20orf30, PLCB1, LOC200261, C20orf112, ADA, PREX1, C21orf34, C21orf42, ERG, ABCG1, MN1, HORMAD2, LOC113826, C22orf1, EFHC2, SYLT4, MGC27005, FHL1, GAB3, and CSF2RA.

### EXAMPLES

The following examples are offered to illustrate, but not to limit, the claimed invention.

Background: Clinical History of Patient P1 and Patient P2 Before and after Gene Therapy

First diagnosis of X-linked chronic granulomatous disease (X-CGD) in patient P1 was done in 1981. He suffered from severe bacterial and fungal infections as well as granuloma of the ureter with stenosis, pyeloplastic operation (1978), liver abscesses (1980), pseudomonassepticemia (1985), candida-oesophagitis (1992), salmonellasepticemia (1993), severe osteomyelitis, spondylitis with epidural and paravertebral abscess and corporectomy (June 2002). Since 2003 severe therapy-resistant liver abscesses (Staph. aureus) were diagnosed. On admission to the hospital in Frankfurt, the patient was treated with clindamycin, cefalexin, cotrimoxazol and itraconazol, the later two as standard long-term prophylaxis. Treatment was changed from clindamycin to rifampicin orally. After gene therapy and resolution of the liver abscesses, rifampicin was removed (day +65) and the patient was kept under standard prophylactic care with itraconazol. During the follow-up and concomitant increase in gene marked cells with effective killing of Aspergillus fumigatus, itraconazol was also removed (day +381). No reappearance of liver abscesses and no positive bacterial culture were observed until the last monitoring time point. The patient had a net weight gain of 10 kg since transplantation and a marked decrease of lung granulomas in the CT scan. Lung function was stable.

First diagnosis of X-CGD for patient P2 was in 1979. He suffered from cervical lymph node abscesses (1983), meningitis (1985), parotis abscesses (1990), two liver abscesses, cervical lymph node abscesses (1991 and 1992), sinusitis maxillaris (1995), bilateral hidradenitis axillaris and pneumonia (2000). Since 2002 he was suffering from bilateral lung aspergillosis with cerebral emboli and formation of a lung cavity. The patient was admitted to the hospital treated by voriconazol and cotrimoxazol. After gene therapy a complete resolution of the aspergillosis was observed, but no improvement in lung function was observed due to excess abuse of nicotine. The patient developed a mycoplasma pneumonia (positive serological IgM titers, no antigen positivity in serum and sputum, negative culture after bronchoalveolar lavage) and sinusitis maxillaris on day +149. He was treated with oral clindamycin for 3 weeks. During gene therapy and busulfan treatment, the voriconazol treatment was changed to liposomal amphotericin B until day +23. Voriconazol treatment was restarted on day +24. No hospital admissions after gene therapy and no positive bacterial cultures were observed. P2 is currently still under cotrimoxazole/voriconazole prophylaxis because the number of oxidase positive cells and the amount of superoxide production per cell were less than 20%. Furthermore, killing of A. fumigatus could not be demonstrated in vitro.

### Example 1

### Description of the Vector and Gene Transfer Protocol for Treatment of the 2 Successfully-Treated CGD Patients Receiving Gene Therapy

For the construction of the retroviral vector SF71gp91^{phox} the pSF71 backbone [Hildinger, M. et al. FMEV vectors: both retroviral long terminal repeat and leader are important for high expression in transduced hematopoietic cells. Gene Ther 5, 1575-1579 (1998), herein incorporated by reference in its entirety] was used, in which the coding region of gp91^{phox} was inserted by standard molecular cloning. In this vector, gp91^{phox} expression is driven by the Friend mink cell Spleen focus-forming virus (SFFV) LTR, which has been shown to be highly active in stem and myeloid progenitor cells [Baum, C. et al. Novel retroviral vectors for efficient expression of the multidrug resistance (mdr-1) gene in early hematopoietic cells. J Virol 69, 7541-7547 (1995), herein incorporated by reference in its entirety]. Vector containing supernatants were obtained from a stable PG13 packaging cell line in X-VIVO10 at a titer of 1×10⁶ TU/ml. CD34+ cells were prestimulated for 36 hours at a density of 1×10⁶ cells/ml in X-VIVO 10 medium+2 mM L-glutamine, supplemented with IL-3 (60 ng/ml), SCF (300 ng/ml), Flt3-L (300 ng/ml), and TPO (100 ng/ml) (Strahtman Biotech, Dengelsberg, Germany) in Lifecell Bags (Baxter). Following prestimulation, cells were adjusted to a density of 1×10⁶ cells/ml in cytokine containing medium as described above. Transduction was performed in tissue culture flasks coated with 5 μg/cm² of CH-296 (Retronectin, Takara, Otsu, Japan) and preloaded with retroviral vector containing supernatant as described previously [Kuhlcke, K. et al. Highly efficient retroviral gene transfer based on centrifugation-mediated vector preloading of tissue culture vessels. Mol Ther 5, 473-478 (2002), herein incorporated by reference in its entirety]. After 24 hours cells were pelleted and cell density was again adjusted to 1×10⁶ cells/ml in cytokine containing medium. Cells were incubated on freshly coated/preloaded flasks for another round of transduction. This procedure was repeated once more for a total of three transduction rounds. 24 hours after the final transduction, cells were harvested and analyzed for phenotype and gene transfer efficiency, transported to the transplantation unit and reinfused into the patients.

End of production materials were also tested for the presence of replication competent retroviruses by the extended XC plaque assay [Cham, J. C. et al. Alteration of the syncytium-forming property of XC cells by productive Moloney leukemia virus infection. Cancer Res 35, 1854-1857 (1975), herein incorporated by reference in its entirety] and by a gag-specific PCR as follows: Primers 5′-AGAGGAGAACGGCCAGTATTG-3′ (SEQ ID NO: 136) and 5′-ACTCCACTACCTCGCAGGCATT-3′ (SEQ ID NO: 137) were used to amplify a 69-bp fragment of the retroviral gag cDNA. Amplification was detected with a FAM-labelled gag-probe (5′-TGTCCGTTTCCTCCTGCGCGG-3′) (SEQ ID NO: 138). The human EPO receptor gene was used as an internal amplification control. PCR reactions were carried out for 40 cycles in a single tube. Each reaction cycle consisted of 15 seconds at 94° C. followed by 1 minute at 60° C.

The pretreatment preparation, treatment, and clinical examination of the 2 successfully treated CGD patients is described further in Ott, M. G. et al. Correction of X-linked chronic granulomatous disease by gene therapy, augmented by insertional activation of MDS1-EV1, PRDM16 or SETBP1. Nat Med 12(4):401-409, (2006), hereby incorporated by reference in its entirety.

### Example 2

### Description of the Gp91^{phox} PCR Method

The ABI PRISM 7700 Sequence Detection System (PE Applied Biosystems, Weiterstadt, Germany) was used to determine the presence of proviral sequences in genomic DNA isolated from the blood and bone marrow cells of patients P1 and P2. The exon 8 primer gp91-f (5′-GGTTTTGGCGATCTC AACAGAA-3′) (SEQ ID NO: 1) and exon 9 primer gp91-r (5′-TGTATTGTCCCACTTCCATTTTGAA-3′) (SEQ ID NO: 2) were used to amplify a 114-bp fragment of the gp91^{phox} cDNA. Amplification was detected with the FAM-labelled probe gp91-p (5′-TCATCACCAAGGTGGTC ACTCACCCTTTC-3′) (SEQ ID NO: 3). The human EPO receptor gene was used as an internal control to quantify the gp91^{phox} reaction. Primers hepo-f (5′-CTGCTGCCAGC TTTGAGTACACTA-3′) (SEQ ID NO: 4) and hepo-r (5′-GAGATGCCAGAGTCAGATACCACAA-3′) (SEQ ID NO: 5) amplified a 138-bp fragment from exon 8 of the EPO-receptor-gene. Amplification was determined by the VIC-labelled probe hepo-p (5′-ACCCCAGCT CCCAGCTCTTGCGT-3′) (SEQ ID NO: 6). Both reactions were carried out in a single tube. The amplification cycle was 15 s at 94° C. followed by 1 min at 60° C. In each experiment, the amplification of DNA generated from HT1080 cells containing a single copy of a gp91^{phox} vector mixed with wild type HT1080 cells in defined ratios was used to quantify the percentage of SF71 gp91^{phox} integrations per human genome. The percentage of transduced cells was estimated from the values obtained from the quantitative PCR (Q-PCR), which represent vector copies per diploid genome, after dividing by two to account for the mean of two proviral copies per transduced cell. Similarly, genomic DNA was isolated from individual bone marrow colonies and analyzed for the presence of vector derived sequences by nested PCR using gp91^{phox} specific primers. The primers used for first PCR (95° C., for 5 min, 95° C. for 1 min, 56° C. for 1 min, 72° C. for 1 min, for 30 cycles) were gpfor01: (5′-TTGTACGTGGG CAGACCGCAGAGA-3′) (SEQ ID NO: 7) and gprev02: (5′-CCAAAGGGCCCATCAACCGCTATC-3′) (SEQ ID NO: 8). Nested PCR was done under similar conditions using the primer combination P8: (5′-GGATAGTGGGTCCCATGTTTCTG-3′) (SEQ ID NO: 9) and R11: (5′-CCGCTATCTTAGGTAG TTTCCACG-3′) (SEQ ID NO: 10). As an internal control the EPO-R gene was amplified in parallel with the primer combination hEpo-F1: (5′-GAGCCGGGGACAGATGATGAGG-3′) (SEQ ID NO: 11) and hEpo-R1: (5′-GCGGCTGGGATAAGGCTGTTC-3′) (SEQ ID NO: 12) for the first PCR reaction and primers hepo-f (SEQ ID NO: 4) and hepo-r (SEQ ID NO: 5) for the nested PCR primers.

### Example 3

### Integration Site Analysis by the Linear Amplification Mediated (LAM)-PCR Method

100 ng of DNA from peripheral blood leukocytes was used for integration site analysis that was performed by LAM PCR as previously described (Schmidt, et al. (2002) Blood 100:2737-2743; Schmidt, et al. (2003) Nature Med. 9:463-468, each of the foregoing which is hereby incorporated by reference in its entirety) but biotinylated primer LTR I (5′>GTT TGG CCC AAC GTT AGC TAT T<3′) (SEQ ID NO: 13) was used for the initial linear amplification of the vector genome junctions. Following magnetic capture, hexa-nucleotide primed double strand synthesis with Klenow polymerase, restriction digest using MseI, HinP1I, or Tsp5091 and ligation of a restriction site complementary linker cassette allowed amplification of the vector genome junctions. For the 1^{st} and 2^{nd} exponential PCR amplification, vector specific primers LTR II (5′>GCC CTT GAT CTG AAC TTC TC<3′) (SEQ ID NO: 14) and LTR III (5′>TTC CAT GCC TTG CAA AAT GGC<3′) (SEQ ID NO: 15) were used in combination with linker cassette specific primers LC I (5′>GAC CCG GGA GAT CTG AAT TC3′) (SEQ ID NO: 16) and LC II (5′>GAT CTG AAT TCA GTG GCA CAG<3′) (SEQ ID NO: 17), respectively. LAM-PCR amplicons were purified, shotgun cloned into the TOPO TA vector (Invitrogen, Carlsbad, Calif.) and sequenced (GATC, Konstanz, Germany). Sequences were aligned to the human genome (hg17, release 35, May 2004) using the UCSC BLAT genome browser (available on the world wide web at ucsc.genome.edu). (See also Table 1.) Relation to annotated genome features were studied with the same tool. Sequences that could not be mapped were either too short (<20 bps, 136 sequences, 15.5% of all obtained sequences), or showed no definitive hit or multiple hits on the human genome (40 sequences, 4.5% of all obtained sequences).

### Example 4

### Qualitative Tracking of Individual Common Insertion Site (CIS) Clones

Individual MDS1/EVI-1, PRDM16, and SETBP1 related insertions were followed over time using clone specific nested primer sets (Perkins, A. S. et al. Evi-1, a murine zinc finger proto-oncogene, encodes a sequence-specific DNA-binding protein. Mol Cell Biol 11, 2665-2674 (1991), hereby incorporated by reference in its entirety). To identify clones with possible predominance, PCR tracking was performed on 10 ng of GenomiPhi™ DNA Amplification Kit (Amersham) pre-amplified DNA from patient peripheral blood leukocytes. 0.5% of the pre-amplified DNA served as template for an initial amplification by PCR with the genomic flanking primer FP1 (SEQ ID NOs: See Table 4) and the vector specific primer LTR I (SEQ ID NO: 13). 2% of this product was applied to a nested PCR with FP2 (SEQ ID NOs: See Table 4) and LTR II (SEQ ID NO: 14) using the same conditions. The products were separated on a 2% agarose gel. Individual ones were purified and sequenced (GATC) (Table 2). Clone specific genomic flanking primers are listed in Tables 3 and 4 (SEQ ID NO: 18 through SEQ ID NO: 135, Table 4). PCR cycling conditions were performed for 35 cycles of denaturation at 95° C. for 45 s, annealing at 56-58° C. for 45 s and extension at 72° C. for 60 s, after initial denaturation for 2 min and before final extension for 5 min.

TABLE 4

| | | | | |
|---|---|---|---|---|
| SEQ ID | Sequence | RefSeq | Primer | |
| NO | Number | Gene | ID | Sequence |

| | | | | |
|---|---|---|---|---|
| 18 | 75917-D12 | PRDM16 | FP1 | 5′>TCGCCGCTGGCCTGCTA |
| | | | | AAT<3′ |
| 19 | 75917-D12 | PRDM16 | FP2 | 5′>CTGCTAAATGAATCTGA |
| | | | | GGG<3′ |
| 20 | 75917-D12 | PRDM16 | FP3 | 5′>CTGCTAAATGAATCTGA |
| | | | | GGG<3′ |
| 21 | 75917-D12 | PRDM16 | FP4 | 5′>AATGAATCTGAGGGCAG |
| | | | | CTG<3′ |
| 22 | 76777-B04 | PRDM16 | FP1 | 5′>TTGCACCTGGAGCTCGG |
| | | | | CTC<3′ |
| 23 | 76777-B04 | PRDM16 | FP2 | 5′>AAGCAGGGCGACAAGAG |
| | | | | GTT<3′ |
| 24 | 76778-G12 | PRDM16 | FP1 | 5′>GTCGTCGTGTTGGTAAT |
| | | | | CCC<3′ |
| 25 | 76778-G12 | PRDM16 | FP2 | 5′>TGAGGGCACTGCTCGTG |
| | | | | TGG<3′ |
| 26 | 75523-G10 | PRDM16 | FP1 | 5′>TAAGGAGCGCGTCGAGG |
| | | | | GGG<3′ |
| 27 | 75523-G10 | PRDM16 | FP2 | 5′>GGCTTCGGCCTCCAACC |
| | | | | CGA<3′ |
| 28 | 76778-G04 | PRDM16 | FP1 | 5′>TTGCGAGCTCCGTGCAG |
| | | | | TTA<3′ |
| 29 | 76778-G04 | PRDM16 | FP2 | 5′>ACAAGATGCCATGTTAA |
| | | | | TTA<3′ |
| 30 | 76777-B11 | PRDM16 | FP1 | 5′>TGCGAGCTCCGTGCAGT |
| | | | | TAC<3′ |
| 31 | 76777-B11 | PRDM16 | FP2 | 5′>TCCAAATAACAAGATGC |
| | | | | CAT<3′ |
| 32 | 75917-B07 | PRDM16 | FP1 | 5′>TAAATAAGTGTTTTCCT |
| | | | | TAC<3′ |
| 33 | 75917-B07 | PRDM16 | FP2 | 5′>TAAGTGTTTTCCTTACG |
| | | | | ACT<3′ |
| 34 | 75917-G07 | PRDM16 | FP1 | 5′>AGAGGCTTCTGTTTCCG |
| | | | | CAG<3′ |
| 35 | 75917-G07 | PRDM16 | FP2 | 5′>TGCTCCCCACCTAACAC |
| | | | | TCG<3′ |
| 36 | 76778-C05 | PRDM16 | FP1 | 5′>TTTATGTTATCGAGGCA |
| | | | | GAA<3′ |
| 37 | 76778-C05 | PRDM16 | FP2 | 5′>ATGTTATCGAGGCAGAA |
| | | | | TTC<3′ |
| 38 | 76778-B07 | PRDM16 | FP1 | 5′>TATGTTATCGAGGCAGA |
| | | | | ATT<3′ |
| 39 | 76778-B07 | PRDM16 | FP2 | 5′>CGATTCAGTGGCAGTGA |
| | | | | GCC<3′ |
| 40 | 76771-H02 | EVI1 | FP1 | 5′>TAGACTGTGACCCTGAA |
| | | | | GAC<3′ |
| 41 | 76771-H02 | EVI1 | FP2 | 5′>ACTAAGGGTGATTTGCT |
| | | | | TTG<3′ |
| 42 | 77110-D02 | EVI1 | FP1 | 5′>GATTAGCTATGTATACT |
| | | | | GCA<3′ |
| 43 | 77110-D02 | EVI1 | FP2 | 5′>GTAATTTGTTACCCTCT |
| | | | | TTA<3′ |
| 44 | 75916-D12 | EVI1 | FP1 | 5′>GTTCTCAGAAACCCAAG |
| | | | | ACA<3′ |
| 45 | 75916-D12 | EVI1 | FP2 | 5′>CAGTGCCTAAGCTGACT |
| | | | | TTG<3′ |
| 46 | 77048-E02 | EVI1 | FP1 | 5′>GTAGATGTTTGGTTTAC |
| | | | | TTC<3′ |
| 47 | 77048-E02 | EVI1 | FP2 | 5′>CACATAGGTGCTTCTGT |
| | | | | ATG<3′ |
| 48 | 79207-B11 | EVI1 | FP1 | 5′>CTTTCATGAGAAACAAG |
| | | | | GCC<3′ |
| 49 | 79207-B11 | EVI1 | FP2 | 5′>GGATTTCAGAACCCTAT |
| | | | | CTT<3′ |
| 50 | 75916-F04 | EVI1 | FP1 | 5′>AGAACTGAGTATTATTA |
| | | | | CTG<3′ |
| 51 | 75916-F04 | EVI1 | FP2 | 5′>ATCAAGAACATCTTGTG |
| | | | | AAT<3′ |
| 52 | 76776-G04 | MDS1 | FP1 | 5′>CTGCCTTCATTGTGTAA |
| | | | | CTG<3′ |
| 53 | 76776-G04 | MDS1 | FP2 | 5′>GTAAGAAGTTAGTGCTC |
| | | | | CAG<3′ |
| 54 | 76776-E04 | MDS1 | FP1 | 5′>GATGGAGTAGAAACTGT |
| | | | | CTG<3′ |
| 55 | 76776-E04 | MDS1 | FP2 | 5′>GTTTGAGCCATGCAAAT |
| | | | | CTG<3′ |
| 56 | 74718-H10 | MDS1 | FP1 | 5′>TAACATAAATAAGTCTT |
| | | | | TAG<3′ |
| 57 | 74718-H10 | MDS1 | FP2 | 5′>CATAAATAAGTCTTTAG |
| | | | | GTT<3′ |
| 58 | 76776-A10 | MDS1 | FP1 | 5′>GGAGACACATCAAGGAA |
| | | | | CTT<3′ |
| 59 | 76776-A10 | MDS1 | FP2 | 5′>ATGTATTGCAACTGGCA |
| | | | | TAG<3′ |
| 60 | 75916-A01 | MDS1 | FP1 | 5′>TAAGGTTACATCCCACA |
| | | | | GCT<3′ |
| 61 | 75916-A01 | MDS1 | FP2 | 5′>CCAGATGAAGTTAGTTT |
| | | | | TTG<3′ |
| 62 | 75916-A01 | MDS1 | FP3 | 5′>CCAGATGAAGTTAGTTT |
| | | | | TTG<3′ |
| 63 | 75916-A01 | MDS1 | FP4 | 5′>AGAAAATGGGTGTATGA |
| | | | | TGA<3′ |
| 64 | 75917-B04 | MDS1 | FP1 | 5′>AATTATACAACATTGGT |
| | | | | GTA<3′ |
| 65 | 75917-B04 | MDS1 | FP2 | 5′>ATGTCACCAATGTAATG |
| | | | | ACA<3′ |
| 66 | 76771-D05 | MDS1 | FP1 | 5′>AGTATTGCATATCTATA |
| | | | | TGA<3′ |
| 67 | 76771-D05 | MDS1 | FP2 | 5′>TCTACACAGTAATGTAT |
| | | | | TTA<3′ |
| 68 | 75916-A08 | MDS1 | FP1 | 5′>CTTCCTCACAGAAGGAT |
| | | | | TGG<3′ |
| 69 | 75916-A08 | MDS1 | FP2 | 5′>TATTGACACCACTTTCT |
| | | | | AGC<3′ |
| 70 | 75916-A08 | MDS1 | FP3 | 5′>TATTGACACCACTTTCT |
| | | | | AGC<3′ |
| 71 | 75916-A08 | MDS1 | FP4 | 5′>TAGGACGATATCAATAC |
| | | | | TTA<3′ |
| 72 | 76776-A11 | MDS1 | FP1 | 5′>TAGATGAAGAAAATTCA |
| | | | | CTC<3′ |
| 73 | 76776-A11 | MDS1 | FP2 | 5′>TTGCCAAGTGTTGAGGT |
| | | | | GCA<3′ |
| 74 | 76776-A11 | MDS1 | FP3 | 5′>TTGCCAAGTGTTGAGGT |
| | | | | GCA<3′ |
| 75 | 76776-A11 | MDS1 | FP4 | 5′>TGAGCGAAAATTGTAGA |
| | | | | ACA<3′ |
| 76 | 78016-F03 | MDS1 | FP1 | 5′>TGAACAAGAGTAGTGTC |
| | | | | ACA<3′ |
| 77 | 78016-F03 | MDS1 | FP2 | 5′>GATGTCAACAGAGCATT |
| | | | | GAG<3′ |
| 78 | 78016-C11 | MDS1 | FP1 | 5′>CGTCTTGTAACTCTCTC |
| | | | | AAG<3′ |
| 79 | 78016-C11 | MDS1 | FP2 | 5′>GCTTGATGTTTAGTCTG |
| | | | | TGC<3′ |
| 80 | 75916-A05 | MDS1 | FP1 | 5′>ACAGGCAATAAAGTTCA |
| | | | | GGA<3′ |
| 81 | 75916-A05 | MDS1 | FP2 | 5′>AGCCCAGGACTCATTTC |
| | | | | TCG<3′ |
| 82 | 75916-A05 | MDS1 | FP3 | 5′>AGCCCAGGACTCATTTC |
| | | | | TCG<3′ |
| 83 | 75916-A05 | MDS1 | FP4 | 5′>GTGTGCCTTGATCGCTC |
| | | | | AAG<3′ |
| 84 | 76776-G11 | MDS1 | FP1 | 5′>GAGCAGTTACAGAGGCT |
| | | | | TGT<3′ |
| 85 | 76776-G11 | MDS1 | FP2 | 5′>CTGCACCAGTAACACAG |
| | | | | TGA<3′ |
| 86 | 77048-C07 | MDS1 | FP1 | 5′>ATACCAACAGGTACGAC |
| | | | | TGG<3′ |
| 87 | 77048-C07 | MDS1 | FP2 | 5′>GTATTCTCAATGATTCC |
| | | | | CCT<3′ |
| 88 | 77512-B07 | SETBP1 | FP1 | 5′>TGCTTTTCTTCAAAGGA |
| | | | | TGG<3′ |
| 89 | 77512-B07 | SETBP1 | FP2 | 5′>AAGGATGGGTTGGAGCG |
| | | | | TTA<3′ |
| 90 | 76778-F12 | SETBP1 | FP1 | 5′>CCGAACTGCACAGCTCA |
| | | | | GCA<3′ |
| 91 | 76778-F12 | SETBP1 | FP2 | 5′>CTCAGCAAAAGCGCCCT |
| | | | | CGC<3′ |
| 92 | 76778-F12 | SETBP1 | FP3 | 5′>CTCAGCAAAAGCGCCCT |
| | | | | CGC<3′ |
| 93 | 76778-F12 | SETBP1 | FP4 | 5′>TCGCCCTCCGCGCGCCG |
| | | | | CCTC<3′ |
| 94 | 76776-E09 | SETBP1 | FP1 | 5′>TAACGCTCCAACCCATC |
| | | | | CT<3′ |
| 95 | 76776-E09 | SETBP1 | FP2 | 5′>AGCATTGATCGGAGAGA |
| | | | | CG<3′ |
| 96 | 75916-G10 | SETBP1 | FP1 | 5′>AGGCAGTAGTGTCGGTT |
| | | | | AAG<3′ |
| 97 | 75916-G10 | SETBP1 | FP2 | 5′>GCTAGGCAAGTGAAGGG |
| | | | | CTG<3′ |
| 98 | 77509-D02 | SETBP1 | FP1 | 5′>CTTCAACCAGCTCCGCC |
| | | | | ATG<3′ |
| 99 | 77509-D02 | SETBP1 | FP2 | 5′>ACCAGTGCCTATTCAAG |
| | | | | CCT<3′ |
| 100 | 79272 F07 | PRDM16 | FP1 | 5′>GGTCCTTTCTAATTGAC |
| | | | | GCG<3′ |
| 101 | 79272 F07 | PRDM16 | FP2 | 5′>TTCAGAGACGCAGCCAC |
| | | | | AGA<3′ |
| 102 | 78373 E04 | PRDM16 | FP1 | 5′>TGGTCTCCTTAGAGGCT |
| | | | | TCT<3′ |
| 103 | 78373 E04 | PRDM16 | FP2 | 5′>GAGGCAGCCACAGAAGG |
| | | | | AGG<3′ |
| 104 | 78166 D04 | PRDM16 | FP1 | 5′>CTGCGTCTCTGAAAGGA |
| | | | | TCC<3′ |
| 105 | 78166 D04 | PRDM16 | FP2 | 5′>AGAAAGGACCCGTTGGC |
| | | | | CAC<3′ |
| 106 | 79275 B07 | PRDM16 | FP1 | 5′>AGGAGTTAAGGAGCGCG |
| | | | | TCG<3′ |
| 107 | 79275 B07 | PRDM16 | FP2 | 5′>CCAACCCGACTTTGTTT |
| | | | | GCG<3′ |
| 108 | 78165 H02 | PRDM16 | FP1 | 5′>TTGCACCTGGAGCTCGG |
| | | | | CTC<3′ |
| 109 | 78165 H02 | PRDM16 | FP2 | 5′>CAAGAGGTTCTGGCTGG |
| | | | | TGG<3′ |
| 110 | 79275 E09 | PRDM16 | FP1 | 5′>AATGCACAGGCCTGCCT |
| | | | | TTA<3′ |
| 111 | 79275 E09 | PRDM16 | FP2 | 5′>CGCTGATTTTCCTCCAG |
| | | | | CGG<3′ |
| 112 | 79275 G07 | EVI1 | FP1 | 5′>GAAGCTATTTCCTTAGA |
| | | | | CAG<3′ |
| 113 | 79275 G07 | EVI1 | FP2 | 5′>TAAGAACGGGACTTGTA |
| | | | | GCC<3′ |
| 114 | 78166 B03 | EVI1 | FP1 | 5′>CTGCCTTTCCACTGATA |
| | | | | GTT<3′ |
| 115 | 78166 B03 | EVI1 | FP2 | 5′>GAAGGAACACACTCCTG |
| | | | | GCC<3′ |
| 116 | 78166 H11 | EVI1 | FP1 | 5′>TGAAAGGGTATGCTTGA |
| | | | | AAG<3′ |
| 117 | 78166 H11 | EVI1 | FP2 | 5′>ACGTCTCTCTGCAAATA |
| | | | | TGA<3′ |
| 118 | 78165 D10 | MDS1 | FP1 | 5′>ACGTAAGACAACTCCAC |
| | | | | AGT<3′ |
| 119 | 78165 D10 | MDS1 | FP2 | 5′>CCACATCAGAGTCAAGA |
| | | | | AGA<3′ |
| 120 | 78165 D10 | MDS1 | FP3 | 5′>CCACATCAGAGTCAAGA |
| | | | | AGA<3′ |
| 121 | 78165 D10 | MDS1 | FP4 | 5′>CTAATTACTGAGATAGC |
| | | | | TCC<3′ |
| 122 | 79275 E08 | MDS1 | FP1 | 5′>CCATTATGTTCCTCATT |
| | | | | GCA<3′ |
| 123 | 79275 E08 | MDS1 | FP2 | 5′>GAGCAAACTTCAAAGGA |
| | | | | AGC<3′ |
| 124 | 79275 E08 | MDS1 | FP3 | 5′>AAGAAGAGGGTGGGCCC |
| | | | | AAG<3′ |
| 125 | 79275 E08 | MDS1 | FP4 | 5′>GTACTTTGTGCCCAACT |
| | | | | TGC<3′ |
| 126 | 78166 B04 | MDS1 | FP1 | 5′>GAATGCTGCAACTGCAA |
| | | | | GGA<3′ |
| 127 | 78166 B04 | MDS1 | FP2 | 5′>CAGTCAGCATGGAAATG |
| | | | | ATT<3′ |
| 128 | 78166 B04 | MDS1 | FP3 | 5′>CAGTCAGCATGGAAATG |
| | | | | ATT<3′ |
| 129 | 78166 B04 | MDS1 | FP4 | 5′>GTCCTCTCTTCATTGTG |
| | | | | TCA<3′ |
| 130 | 78166 D08 | MDS1 | FP1 | 5′>GCTCTCCTTCAGCATGT |
| | | | | CAA<3′ |
| 131 | 78166 D08 | MDS1 | FP2 | 5′>GAGATTCACACAGTAAA |
| | | | | AGA<3′ |
| 132 | 78166 E03 | MDS1 | FP1 | 5′>CAGGCTAACTTCTCGAC |
| | | | | TCT<3′ |
| 133 | 78166 E03 | MDS1 | FP2 | 5′>CAACTGGCCTGAATTAG |
| | | | | AGT<3′ |
| 134 | 78166 H03 | MDS1 | FP1 | 5′>CAGGACCCTTCACGGAT |
| | | | | ACC<3′ |
| 135 | 78166 H03 | MDS1 | FP2 | 5′>GGCATAGCATTTGCATA |
| | | | | TAA<3′ |

### Example 5

### Quantitative Competitive (QC) PCR Analysis

To calculate the proportional contribution of individual predominant clones to gene corrected myelopoiesis, an internal standard (IS) PCR template revealing a 26-bp deletion within the 5′LTR vector sequence was generated for each vector genome junction of interest [Hoyt, P. R. et al. The Evi1 proto-oncogene is required at midgestation for neural, heart, and paraxial mesenchyme development. Mech Dev 65, 55-70 (1997), hereby incorporated by reference in its entirety]. The coamplification of a certain amount of ‘wild-type’ (WT) patient DNA with a defined copy number of IS allowed estimation of the abundance of the specific integrant in the patient DNA. QC-PCR was performed with defined dilutions of IS (50 copies and 500 copies) added to 50 ng of patient DNA. Using vector primer LTR I (SEQ ID NO: 13) and genomic flanking primer FP2 (SEQ ID NOs: See Table 4), the templates were coamplified with 35 PCR cycles (denaturation at 95° C. for 45 s, annealing at 54-60° C. for 45 s, extension at 72° C. for 60 s) after initial denaturation for 2 min and before final extension for 5 min. 0.1-2% of the reaction product was used as template for a second nested PCR, which was performed for 35 cycles with the same parameters as for the first PCR with primers LTR II (SEQ ID NO: 14) and FP3 (SEQ ID NOs: See Table 4). QC-PCR products were separated on a 2% agarose gel (FIGS. 18, 19 and Table 2). Primers used for the generation of IS and further QC-PCR are listed in Tables 3 and 4 (SEQ ID NO: 18 through SEQ ID NO: 135, Table 4).

### Example 6

### Methods of RNA Extraction and Analysis

Total RNA was extracted from bone marrow derived from patient 1 and a healthy donor with the RNeasy Mini Kit (Qiagen). cDNA was synthesized using the First Strand cDNA Synthesis Kit (Amersham) with whole RNA extracted and 0.2 μg of Not I d(T)18 primer (5′>AAC TGG AAG AAT TCG CGG CCG CAG GAA<3′) (SEQ ID NO: 139). A 35 cycle actin PCR was carried out as a loading control using primers actin-1 (5′-TCCTGTGGCATCCACGAAACT-3′) (SEQ ID NO: 140) and actin-2 (5′-GAAGCATTTGCGGTGGAC GAT-3′) (SEQ ID NO: 141) for 5 min at 95° C., 1 min at 95° C., 1 min at 58° C., 1 min at 72° C., and 10 min at 72° C.

EVI-1 and MDS1-EVI-1 transcripts were detected by PCR with primers EVI1-ex5-F2 (5′-TGGAGAAACACATGCTGTCA-3′) (SEQ ID NO: 142) and EVI1-ex6-R2 (5′-ATAAAGGGCTTCACA CTGCT-3′) (SEQ ID NO: 143). To amplify only PR domain positive MDS1-EVI-1 transcripts, cDNA was subjected to a 36 cycle PCR using primers MDS1-ex2-F1 (5′-GCCACATCCAGT GAAGCATT-3′) (SEQ ID NO: 144) and EVI1-ex2-R1 (5′-TGAGCCAGCTTCCAACATCT-3′) (SEQ ID NO: 145). 2% of the PCR product was introduced into a second PCR using nested primers MDS1-ex2-F2 (5′-AGGAGGGTTCTCCTTACAAA-3′) (SEQ ID NO: 146) and EVI1-ex2-R2 (5′-TGACTGGCATCTATG CAGAA-3′) (SEQ ID NO: 147).

To define the expression of PRDM16, a fragment of the PR domain was amplified using primer MEL1PR-F1 (5′-CTGACGGACGTGGAAGTGTCG-3′) (SEQ ID NO: 148) with MEL1PR-R1 (5′-CAGGGGGTAGACGCCTTCCTT-3′) (SEQ ID NO: 149), which hybridized in exon 3 and exon 5, respectively. 2% of the PCR product was amplified in a second PCR with primers MEL1PR-F2 (5′-TCTCCGAAGACCTGGGCAGT-3′) (SEQ ID NO: 150) and MEL1PR-R2 (5′-CACCTG GCTCAATGTCCTTA-3′) (SEQ ID NO: 152). Fragments of both the PR-containing and the non PR-domain containing form of PRDM16 were amplified using primer MEL1N-F1 (5′-CCCCAGATCAGCCAACTCACCA-3′) (SEQ ID NO: 152) and MEL1N-R1 (5′-GGTGCCGGTCCAGGT TGGTC-3′) (SEQ ID NO: 153). Nested PCR was performed with 2% of the product and primer MEL1N-F2 (5′-ACACCTGAGGACGCACACTG-3′) (SEQ ID NO: 154) and MEL1N-R2 (5′-GGTTGCACAGGT GGCACTTG-3′) (SEQ ID NO: 155). Expression level of SETBP1 was analyzed using primers SETBP-F1 (5′-TAAAAGTGGACCAGACAGCA-3′) (SEQ ID NO: 156) and SETBP-R1 (5′-TCACGAAGTTG TTGCCTGTT-3′) (SEQ ID NO: 157).

To assign whether there are fusion transcripts between the vector LTR and MDS1, EVI-1, or PRDM16, the primer U5 IV (5′>TCC GAT AGA CTG CGT CGC<3′) (SEQ ID NO: 160) together with primer EVI-ex2-R1, MDS1-ex2-F1, or MEL1N-R1. Nested PCR was performed with 2% PCR product and primer U5 VI (5′>TCT TGC TGT TTG CAT CCG AA<3′) (SEQ ID NO: 161) was used together with primer EVI1-ex2-R2 (SEQ ID NO: 147), MDS1-ex2-F2 (SEQ ID NO: 146), or MEL1N-R2 (SEQ ID NO: 155). Additionally, nested PCR was carried out with LTR I (SEQ ID NO: 13) and MEL1-PR-F1 (SEQ ID NO: 148). 2% of the product was amplified with primer LTR II (SEQ ID NO: 14) and MEL1PR-F2 (SEQ ID NO: 150). 36 cycle PCRs were accomplished with 3.33% of whole cDNA from patient 1 and 0.33% of whole cDNA from the normal donor for 2 minutes at 95° C., 45 seconds at 95° C., 45 seconds at 54° C., 1 minute at 72° C., and 5 minutes at 72° C. A 35 cycle actin PCR was carried out as a loading control with 0.0002-0.008% of cDNA and primers actin-1 (5′TCC TGT GGC ATC CAC GAA ACT 3′) (SEQ ID NO: 140) and actin-2 (5′ GAA GCA TTT GCG GTG GAC GAT 3′) (SEQ ID NO: 141) for 5 minutes at 95° C., 1 minute at 95° C., 1 minute at 58° C., 1 minute at 72° C., and 10 minutes at 72° C.

### Example 7

### Colony Assay Methods

Bone marrow mononuclear cells (1-5×10⁴) or CD34+ purified cells (1-5×10³) were plated on methylcellulose in the presence or absence of cytokines (50 ng/ml hSCF, 10 ng/ml GM-CSF, 10 ng/m hIL3 and 3 U/ml hEpo) (MethoCult, Stem Cell Technologies, Vancouver, Canada). Colony growth was evaluated after 14 days.

### Example 8

### Method to Detect gp91^{phox} Cell Surface Expression

Heparinized whole blood (100 μl) was incubated with the murine monoclonal antibody 7D5 [Nakamura, M. et al. Monoclonal antibody 7D5 raised to cytochrome b558 of human neutrophils: immunocytochemical detection of the antigen in peripheral phagocytes of normal subjects, patients with chronic granulomatous disease, and their carrier mothers. Blood 69, 1404-1408 (1987), herein incorporated by reference in its entirety] or an IgG1 isotype control (Becton Dickinson, San Jose, Calif.) for 20 minutes. After washing, samples were stained with FITC-goat (Jackson ImmunoResearch, West Grove, Pa.,) or APC-goat (Caltag Laboratories, Burlingame, Calif.) anti-mouse antibodies. Lineage markers were determined using monoclonal antibodies against CD3 (HIT3a), CD15 (HI98) and CD19 (4G7). After erythrocyte lysis, stained cells were washed, fixed, and analyzed on a FACSCalibur (Becton Dickinson, San Jose, Calif.).

### Example 9

### Killing Assay Methods

Neutrophils obtained either from an untreated CGD patient, or healthy donors were incubated with the E. coli strain ML-35, which lacks the membrane transport protein lactose permease and constitutively expresses β-galactosidase (β-Gal). Engulfment of E. coli mL-35 by wild type neutrophils is followed by perforation of the bacterial cell wall and accessibility to β-Gal, which is subsequently inactivated by reactive oxygen species [Hamers, M. N. et al. Kinetics and mechanism of the bactericidal action of human neutrophils against Escherichia coli. Blood 64, 635-641 (1984), herein incorporated by reference in its entirety]. 2×10⁹ E. coli/ml were opsonized with 20% (v/v) Octaplas® (Octapharma AG, Lachen, Switzerland) for 5 min at 37° C. Opsonized E. coli (final concentration 0.9×10⁸/ml) were added to granulocytes (0.9×10⁷/ml) obtained from healthy donors or X-CGD patients after gene therapy. At defined time points granulocytes were lysed with 0.05% saponin (Calbiochem, Darmstadt, Germany) and samples were incubated with 1 mM ortho-nitrophenyl-βD-galactopyranoside (Sigma-Aldrich, Seelze, Germany) at 37° C. for 30 min. β-galactosidase activity was followed by standard procedures at 420 nm.

The Aspergillus fumigatus killing assay was conducted as described by Rex et al. [Rex, J. H. et al. Normal and deficient neutrophils can cooperate to damage Aspergillus fumigatus hyphae. J Infect Dis 162, 523-528 (1990), herein incorporated by reference in its entirety] with minor modifications. Briefly, Aspergillus spores were seeded in 12 well plates at a density of 5×10⁴ spores per well in Yeast nitrogen with amino acids (Sigma-Aldrich, Seelze, Germany). Hyphae were opsonized with 8% Octaplas® (Octapharma AG, Lachen, Switzerland) for 5 min at room temperature. Subsequently, 1×10⁶ healthy granulocytes or 4×10⁶ neutrophils from patient P1 were added. Following incubation at 37° C., granulocytes were lysed at defined time points in 0.5% aqueous sodium deoxycholate solution for 5 min at room temperature. The mitochondrial activity of the remaining adherent hyphae was monitored by an MTT assay as described [Rex et al. 1990, supra, herein incorporated by reference in its entirety].

### Example 10

### Transmission Electron Microscopy Methods

For evaluation of E. coli killing 5×10⁷ opsonized E. coli were incubated with 5×10⁶ granulocytes in HBSS+Ca/Mg containing 2% human albumin in a water bath shaker at 37° C. for 2.5 h. The cells were harvested by centrifugation and fixed in 2.5% glutaraldehyde in PBS at room temperature for 30 min. For the evaluation of Aspergillus fumigatus killing, 3×10⁵ Aspergillus spores were seeded in a 4 cm petri dish in Yeast Nitrogen Base with amino acids (Sigma). Germination was induced by 6 h incubation at 37° C. followed by decelerated growth at room temperature over night. Hyphae were washed in HBSS+Ca/Mg and opsonized with 8% Octaplas® (Octapharma AG, Lachen, Switzerland) in HBSS+Ca/Mg containing 0.5% human albumin for 5 min at room temperature. The opsonized hyphae were incubated with 3×10⁶ granulocytes in HBSS+Ca/Mg containing 0.5% human albumin for 2 h at 37° C. Fixation was carried out by direct addition of glutaraldehyde to a final concentration of 2.5%. Glutaraldehyde fixed samples were washed three times in PBS, fixed in 2% osmium tetroxide in PBS for 30 minutes, and dehydrated in ethanol followed by embedding in Epon and polymerization at 60° C. for 2 days. Ultrathin sections of 60 nm were prepared using an Ultramicrotome (Ultracut E, Reichert). The sections were then post-stained with 5% aqueous uranyl acetate for 30 min and lead citrate for 4 min, and examined on a Philips CM 12 transmission electron microscope.

### Example 11

### Immune Reconstitution Assay Methods

Immune reconstitution was monitored by four-color-flow cytometric assessment of T cell subsets, NK cells and B cells in peripheral blood (PB) samples on a Coulter Epics XL. Samples were labelled with the 45/4/8/3 or 45/56/19/3 tetraChrome reagents from Coulter (Krefeld, Germany). All antibodies were obtained from Coulter Immunotech (Marseilles, France). The percentages of cell subtypes determined in these analyses were used to calculate the absolute cell counts in a dual-platform approach.

### Example 12

### Assay Methods for Granulocyte Function

Reconstitution of NADPH oxidase activity in neutrophils after gene therapy was assessed by oxidation of dihydrorhodamine 123 [Vowells, S. J., Sekhsaria, S., Malech, H. L., Shalit, M. & Fleisher, T. A. Flow cytometric analysis of the granulocyte respiratory burst: a comparison study of fluorescent probes. J Immunol Methods 178, 89-97 (1995), herein incorporated by reference in its entirety], reduction of nitrobluetetrazolium13, reduction of cytochrome C [Mayo, L. A. & Curnutte, J. T. Kinetic microplate assay for superoxide production by neutrophils and other phagocytic cells. Methods Enzymol 186, 567-575 (1990), herein incorporated by reference in its entirety] and flavocytochrome b spectral analysis [Bohler, M. C. et al. A study of 25 patients with chronic granulomatous disease: a new classification by correlating respiratory burst, cytochrome b, and flavoprotein. J Clin Immunol 6, 136-145 (1986), herein incorporated by reference in its entirety] according to standard protocols.

### Example 13

### PET/CT-Scanning Methods

Whole body positron emission tomography (PET) using fluorine-18-fluoro-2-deoxy-D-glucose (FDG) was performed simultaneously and fused with computed tomography (CT) scans. Transmission scanning began immediately after the administration of at least 350 MBq of FDG, emission scanning followed 40 min later.

### Example 14

### Clinical Parameters After Gene Therapy

### BM Cellularity

Bone marrow aspirates of both patients were routinely examined at several time points (P1: days +122, +192, +241, +381; P2: days +84, +119, +245). The following analyses were done: morphology (Pappenheim staining) was normal at all time points and showed a completely normal hematopoiesis, normal cellularity, normal megakaryo-, erythro- and granulopoiesis and no signs of leukemia. One example each is described as such: P1 day +381: megakaropoiesis normal, X-cell 1%, promyelocytes 8%, myelocytes 16%, metamyelocytes and bands 14%, segmented 15%, eosinophils 6%, basophils 1%, monocyte 3%, erythroblasts 21%, plasma cells 2%, lymphoids 12%. P2 day +245: megakaryopoiesis normal, promyelocytes 10%, myelocytes 19%, metamyelocytes and bands 12%, segmented 11%, eosinophils 4%, basophils 1%, monocytes 3%, erythroblast 26%, plasma cells 4%, lymphoids 10%.

### Example 15

### Clinical Parameters After Gene Therapy

### CFU-C Content

Bone marrow aspirates were taken at days +122, +192, +241 and +381 for P1 and at days +84, +119 and +245 for P2. On each occasion a bone marrow total BM mononuclear cells were plated on methylcellulose (Methocult, Stem Cells Technologies) and colony formation was assessed 14 days later. Table 5 shows a summary of these data.

TABLE 5

| | CFY-GM per 10⁵ cells | BFU-E per 10⁵ cells |
|---|---|---|
| P1 | | |
| Day +122 | 25 | 24 |
| Day +192 | 25 | 33 |
| Day +241 | 49 | 55 |
| Day +381 | 70 | 133 |
| Day +381 CD34+ (10³) | 29 | 60 |
| P2 | | |
| Day +84 | 49 | 88 |
| Day +119 | 73 | 72 |
| Day +245 | 153 | 52 |
| Day +245 CD34+ (10³) | 42 | 12 |

### Example 16

### Clinical Parameters After Gene Therapy

### Immunophenotyping Methods

Immunophenotyping of bone marrow cells performed by FACS analysis with antibodies against CD19, CD10, CD10/CD19, CD34, CD33 and CD34/CD33 showed no abnormal expression profile or cell counts in either patient at any time.

### Example 17

### Clinical Parameters After Gene Therapy

### Immunostaining Methods

Immunohistostaining of bone marrow biopsies for CD10, CD34, CD117, CD3, and CD20 was performed at day +381 (P1) and day +491 (P2). No infiltration of blast cells, no myelo- or lymphoproliferative disease and no myelodyplastic syndrome were seen in these preparations.

### Example 18

### Clinical Parameters After Gene Therapy

### BM Cytogenetics Analysis

Cytogenetic analysis were performed at the Department of Molecular Pathology, University Medical School, Hannover, Germany under the direction of Prof. Dr. med. B. Schlegelberger. The following samples were analyzed: P1: day +241 (16 metaphases), day +381 (18 metaphases); P2 day +119 (15 metaphases), day +245 (21 metaphases). In all cases a normal karyotype was observed.

### Example 19

### Clinical Parameters After Gene Therapy

### T-Cell Function Analysis

Mononuclear cells obtained at different time points from P1 and P2 were stimulated with diverse mitogens and antigens. Proliferative responses were assayed by ³H-Thymidine incorporation. The ratio of ³H-Thymidine incorporation in mitogen- or antigen stimulated vs. non-stimulated cells is given in Table 6 as a quotient. In all cases, robust incorporation of ³H-Thymidine were observed, indicating that the mitogen and antigen responses of patient lymphocytes are within the range of age-matched healthy individuals. Also, immunoscope analysis of Vβ T lymphocytes at day +245 (P1) and day +491 (P2) showed normal T cell receptor repertoires in both patients.

TABLE 6

| Lymphocyte function | Before | Quotient | Quotient | |
|---|---|---|---|---|
| P1 | GT | day +53 | day +597 | Control |
| Mitogens | | | | |
| PHA | 302 | 167-183 | 57-59 | >30 |
| Staphylococcus Enterotoxin | 136 | 54 | >30 | |
| Anti-CD3 | 109 | 52 | >30 | |
| PMA + Ionomycin | 109 | 32-36 | >30 | |
| Antigens | | | | |
| Candida albicans | 12-17 | 164-175 | >10 | |
| Cytomegalovirus | 14-18 | 2 | >10 | |
| Tuberculin (purified protein | 17 | 183 | >10 | |
| derivate) | | | | |
| Tetanus | 63 | 22-31 | 78-88 | >10 |

| Lymphocyte function | Before | Quotient | |
|---|---|---|---|
| P2 | GT | day +50 | Control |
| PHA | 482-514 | 114-152 | >30 |
| Staphylococcus Enterotoxin | 370 | 283 | >30 |
| Anti-CD3 | 496 | 210 | >30 |
| PMA + Ionomycin | 506 | 95 | >30 |

### Example 20

### Clinical Parameters After Gene Therapy

### Antibody Production Analysis

Among others normal levels of IgG, IgA, IgM, IgG1, IgG2, IgG3 and IgG4 were found. Examples of plasma protein levels are shown below at days +546 (P1) and day +489 (P2) in Table 7.

TABLE 7

| P1 | Before GT | After GT day +546 | Control range |
|---|---|---|---|
| IgG | 995 mg/dl | 1140 mg/dl | 700-1600 |
| IgA | 218 mg/dl | 364 mg/dl | 70-400 |
| IgM | 143 mg/dl | 57 mg/dl | 40-230 |
| P2 | Before GT | After GT day +489 | Control range |
| IgG | 1678 mg/dl | 1140 mg/dl | 700-1600 |
| IgA | 537 mg/dl | 383 mg/dl | 70-400 |
| IgM | 254 mg/dl | 87.2 mg/dl | 40-230 |

Similarly, IgG antibodies against Tetanus Toxoid (610 U/l), Diphteria Toxoid (270 U/l) and Hemophilus influenzae Type B (3.10 μg/ml) were detected at day 597 in serum samples of P1.

### Example 21

### Mouse Integration and Transplantation Data Related to the Clinical Study

To create immortal mouse cell clones, bone marrow cells obtained from C57BL/6-Ly5.1⁺ mice were expanded for 2 days in the presence of DMEM plus 15% heat-inactivated FBS, 10 ng/ml IL-6, 6 ng/ml IL-3, and 100 ng/ml SCF. Expanded cells were subsequently transduced by co-culture on top of GP+E86 cells stably expressing MSCVneo. After transduction, cells were cultured in IMDM with 20% heat-inactivated horse serum plus 100 ng/ml SCF and 10 ng/ml IL-3, or 100 ng/ml SCF and 30 ng/ml FLT3L. More than 80 immortal cell clones were generated after retroviral transduction of murine bone marrow cells in the presence of SCF and IL3, of which some have been maintained in culture for more than 1.5 years. The majority of these clones had a phenotype similar to committed immature myeloid progenitors and were still IL-3 dependent. All karyotypes were found to be normal. Spontaneous differentiation of the cultures yielded neutrophils (10-40%) and macrophages (1-5%). 95% of cells could be differentiated into neutrophils in response to G-CSF, whereas GM-CSF treatment induced differentiation into macrophages (30%) and neutrophils (70%). Addition of PMA induced 50-70% of cells to differentiate into macrophages. Integration sites were analyzed in 37 clones, demonstrating between 1 to 7 integrants per cell. 7 cell clones showed integrants in the Evi1 gene locus, 13 in the Prdm16 gene region and 1 in Setbp1. Northern analysis showed that expression of Evi1 and Prdm16 was mutually exclusive [Du, Y., Jenkins, N. A. & Copeland, N. G. Insertional mutagenesis identifies genes that promote the immortalization of primary bone marrow progenitor cells. Blood 106, 3932-3939 (2005), herein incorporated by reference in its entirety].

The engraftment potential of these immortalized cell lines was also tested. 2-8×10⁶ Ly5.1⁺ cells from Evi1 (two clones), Prdm16 (one) and Setbp1 (one) immortalized cell lines, together with 5×10⁵ unirradiated C57BL/6-Ly5.2⁺ supporting bone marrow cells, failed to engraft lethally irradiated C57BL/6-Ly5.2⁺ mice.

Further, 10 immortalized early hematopoietic progenitor cell clones were produced by retroviral transduction in the presence of SCF and FLT3 ligand. Of these, one (SF-1) revealed a very immature phenotype (Sca-1−, 50% c-kit+) with lymphomyeloid differentiation capacity and an integration in Setbp1. In contrast to the immortalized clones with the committed myeloid progenitor phenotype, transplantation of 2.5-5.6×10⁶ Ly5.1⁺ SF-1 cells resulted in a leukemic phenotype. All eleven hosts died of leukemia 56-118 days post transplant. Secondary recipients of 1×10⁶ leukemic cells developed leukemias 30 days after transplantation. This SF-1 cell line revealed two integrants, one located at an unknown gene locus (without abnormal gene expression) and one in intron 1 of Setbp1. The leukemic potential of SF-1 cells is very likely related to the immature phenotype of the clone (engraftment and self-renewal capacity). This knowledge can be used to develop assays that evaluate the therapeutic value of gene-modified cells against its potential risks in clinical use. For example, such assays can be used to screen gene-modified cells in order to eliminate those clones that exceed a specified risk threshold for clinical therapies. In summary, immortalized early hematopoietic progenitor cells induced leukemias in transplanted hosts whereas immortalized immature myeloid cells did not.

In the clinical study, no SETBP1 integrant was detected in patient P2 (and no SETBP1 overexpression). In contrast, seven integrants in SETBP1, six located about 20 kb upstream and one in intron 1 of the gene, were detected in patient P1. The position of the integrant in intron 1 was similar to the two integrants found in the mouse study. This particular clone (77509D02) was detected only once by LAM-PCR in peripheral blood of P1 at day +241, but was not detected at any other time point by tracking PCR (Tables 1 and 2).

### Example 22

### Transduction and Busulfan Conditioning of Patients

G-CSF mobilized peripheral blood CD34+ cells were collected from two X-CGD patients aged 26 (patient P1) and 25 years (patient P2), transduced with a monocistronic gammaretroviral vector expressing gp91^{phox} (SF71 gp91^{phox}) and reinfused 5 days later (Example 1). Transduction efficiency was 45% for P1 and 39.5% for P2 as estimated by gp91^{phox} expression (Example 2). The proviral copy number was 2.6 (P1) and 1.5 (P2) per transduced cell. The number of reinfused CD34+/gp91+ cells per kg was 5.1×10⁶ for P1 and 3.6×10⁶ for P2. Prior to reinfusion, liposomal busulfan (L-Bu) was administered intravenously on days −3 and −2 every 12 hours at a dose of 4 mg/kg/day. Liposomal busulfan conditioning was well tolerated by both patients P1 and P2. With the exception of a grade I mucositis from day +11 to day +17 observed in P1, no other non-hematological toxicities were observed.

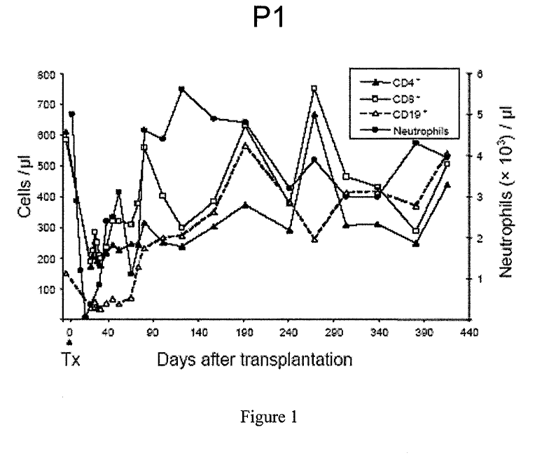

Both patients experienced a period of myelosuppression (neutrophil nadir for P1: day +14 and for P2: day +15) with absolute neutrophil counts (ANC) below 500 cells per μl between days +12 and +21 (P1) and days +13 and +18 (P2) (FIGS. 1,2). Severe lymphopenia (CD4+ counts <200/μl) was observed in P1 between days +21 and +32, while lymphopenia in P2 was observed only at day +17 (FIGS. 1,2). Cell counts recovered gradually to the normal values observed prior to busulfan conditioning (P1: 476 CD4+ cells/μl, age 19; P2: 313 CD4+ cells/μl, day −28). Similar results were observed for CD8+ and CD19+ cells (FIGS. 1,2) (Example 11).

### Example 23

### Engraftment of Gene-Modified Cells

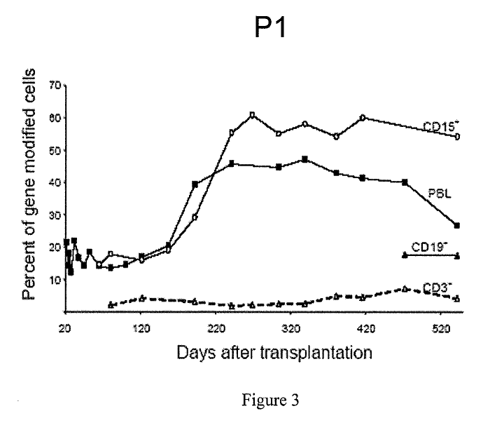

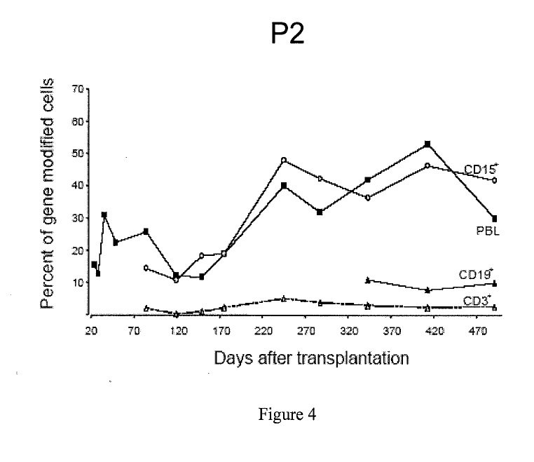

Gene-modified cells were detected in peripheral blood leukocytes (PBL) from patient P1 at levels between 21% (day +21) and 13% (day +80) (Example 2). From day 157, a continuous increase in gene-marked cells was observed until day +241. At this point, 46% of total leukocytes were positive for vector encoded gp91^{phox}. The percentage of gene-marked cells remained at this level until day +381 and decreased thereafter to 27% at day 542 (FIG. 3). A similar result was observed in patient P2. The level of gene marked leukocytes fluctuated between 31% (day +35) and 12% (day +149). Thereafter, an increase in gene-marked cells was observed with 53% of the patient leukocytes containing vector-derived sequences at day +413, which decreased again to 30% at day +491 (FIG. 4).

Vector-containing cells were found predominantly in the myeloid fraction. The level of gene marking in the granulocytes of P1 increased from 15% (day +65) to 55% (day +241) and fluctuated thereafter between 60% (day +269) and 54% (day +542) (FIG. 3). Similar results were observed for P2. While 15% of the granulocytes were marked at day +84, 48% of the granulocytes contained vector-derived sequences at day +245 and fluctuated thereafter between 36% (day +343) and 42% (day +491) (FIG. 4). In both patients the level of gene marking in CD3+ cells remained low (range, 2%-7% (P1) and 0.4%-5% (P2)). In contrast, gene marking levels in isolated CD19+ cells of P1 (purity >98%) were 18% (day +472) and 17% (day +542) (FIG. 3), while in B-cells of P2 (purity >94%) these values fluctuated between 11% (day +343) and 10% (day +491) (FIG. 4).

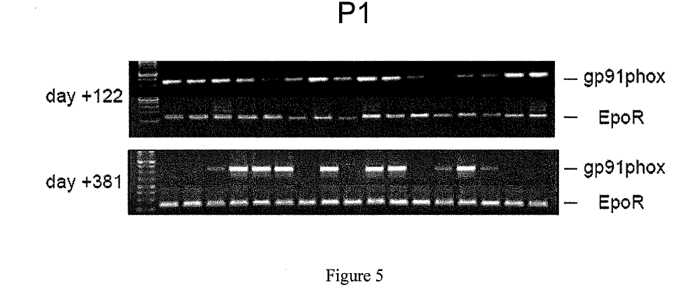

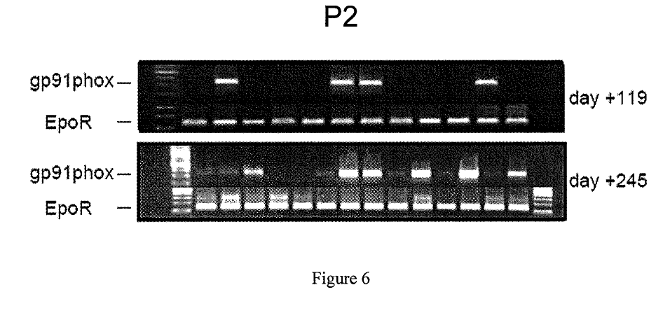

Gene marking in bone marrow hematopoietic progenitor cells was estimated from the number of vector-positive colony-forming cells (CFC). Gene-marked CFCs were detected at a frequency of 68.8% (day +122) and 58.8% (day +381) for patient P1 (FIG. 5), while these values were 33.3% (day +119) and 42.8% (day +245) for patient P2 (FIG. 6). Vector-derived sequences were detected both in colony-forming units-granulocyte-macrophage (CFU-GM; range, 63.2%-76.9% (P1) and 25.0%-6.6% (P2)) and burst-forming units-erythrocyte (BFU-E; range, 50.0%-75% (P1) and 20%-40.0% (P2)) colonies, indicating effective gene marking in common myeloid progenitors with long-term engraftment capacities or in hematopoietic stem cells (HSCs).

### Example 24

### Expansion of Hematopoietic Cells in a Patient by Reinfusing Cells Transfected with a Retroviral Vector

Cells are isolated from a cell sample taken from a patient in need of blood cells. A retroviral vector is prepared. A cell culture is prepared in the presence of permissive cytokines. The cells are allowed to proliferate. When ex vivo expansion is required, cells are kept in culture in the presence of the same or a different set of cytokines or growth factors, e.g. to induce proliferation only at the stem cell stage, or only at a lineage differentiated stage, e.g. myelopoiesis or thrombopoiesis. The cells are prepared for reinfusion to the patient by washing in PBS to remove cell culture components, followed by sorting of cells according to phenotype. The cells are reinfused to the patient. The patient's cell count is taken weekly. By this method, the patient's blood cell count improves.

### Example 25

### Expansion of Hematopoietic Cells In Vitro by Upregulating EVI-1 or PRDM16 Expression

The human EVI-1 nucleic acid sequence, operably linked to a tetracycline-inducible promoter, is inserted into a plasmid vector sequence using known molecular techniques, and is then transfected to a hematopoietic cell culture. The cell culture is allowed to proliferate as described in Example 24 for a 2 week period in the presence of the inducer agent. The cells are then counted and characterized using cell-type specific markers.

### Example 26

### Administration of EVI-1 Expanded Hematopoietic Cells to Patient in Need of Treatment

Cells are reinfused intravenously, directly into the bone marrow or delivered to specific target tissues by direct application or injection in appropriate media, e.g. PBS.

### Example 27

### Administration of EVI-1 to Hematopoietic Cells In Vivo Using Nucleic Acid Vector

A patient in need of expansion of hematopoietic cells is treated with an injection of purified nucleic acid vector containing a nucleic acid sequence encoding EVI-1, operably linked to an inducible promoter. Once in a suitable hematopoietic cell, the nucleic acid integrates into the chromosomal DNA of the patient and/or is transcribed after the inducing agent is provided to the patient orally for 1 year. The hematopoietic cells are capable of in vivo expansion by this method, and the patient health improves.

### Example 28

### Administration of an Agent that Upregulates EVI-Related Genes in a Cell Culture

Cells are isolated from a patient in need of treatment. An agent that upregulates endogenous EVI-1 expression is added to a cell culture, such as an upstream regulator of EVI-1 expression. Cell count is measured daily. After several days, the agent is removed from the culture, the expanded cells are washed and reinfused into the patient.

### Example 29

### Expansion of Hematopoietic Cells In Vitro by Upregulating SETBP1 Expression

The human SETBP1 nucleic acid sequence, operably linked to a steroid hormone inducible promoter, is inserted into an integrating vector sequence using known molecular techniques, and is then transfected to a hematopoietic cell culture. The cell culture is allowed to proliferate as described in Example 6 for one week in the presence of a steroid inducer agent. The cells are then counted and characterized using cell-type specific markers.

### Example 30

### Administration of SETBP1 Expanded Hematopoietic Cells to Patient in Need of Treatment

The desired cells are isolated from the culture described in Example 11, and are washed in PBS. The cells are then reinfused directly into the bone marrow of the patient. By use of this method, the patient hematopoietic cell count improves, and the patient health improves.

### Example 31

### Administration of PRDM16 to Hematopoietic Cells In Vivo Using Nucleic Acid Vector

A patient in need of expansion of hematopoietic cells is treated with an injection of purified nucleic acid vector containing a nucleic acid sequence encoding PRDM16, operably linked to an inducible promoter. Once in a suitable hematopoietic cell, the nucleic acid integrates into the chromosomal DNA of the patient and/or gets transcribed after the inducing agent is provided to the patient orally for 1 year. The hematopoietic cells are capable of in vivo expansion by this method, and the patient health improves.

### Example 32

### Administration of an Agent that Upregulates PRDM16 Genes in a Cell Culture

Cells are isolated from a patient in need of treatment. An agent that upregulates endogenous PRDM16 expression is added to a cell culture, and the cells are allowed to proliferate for 9 days. Cell count is measured daily. After 9 days, the agent is removed from the culture, the expanded cells are washed and reinfused into the patient. By use of this method, the patient health improves.

One skilled in the art will appreciate that these methods and devices are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

It will be apparent to one skilled in the art that varying substitutions and modifications may be made to the invention disclosed herein without departing from the scope and spirit of the invention.

Those skilled in the art recognize that the aspects and embodiments of the invention set forth herein may be practiced separate from each other or in conjunction with each other. Therefore, combinations of separate embodiments are within the scope of the invention as disclosed herein.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

In addition, where features or aspects of the invention are described in terms of Markush groups, those skilled in the art will recognize that the invention is also thereby described in terms of any individual member or subgroup of members of the Markush group.

What is claimed is:

1. A method of expanding cells, comprising:
obtaining at least one cell from a patient;
contacting said cell with a retroviral or nonintegrating vector, such that said vector enters said cell and promotes proliferation, persistence, or selective advantage of the cell;
allowing the cell to proliferate;
introducing a plurality of proliferated cells into said patient; and
allowing said proliferated cells to expand further in the patient.

2. The method of claim 1, wherein said cell is a cell selected from the group consisting of a hematopoietic progenitor cell, a hematopoietic stem cell, and a stem cell.

3. The method of claim 2, wherein said method is used to treat a patient with a hematopoietic or other treatable disease.

4. The method of claim 1, wherein the vector further comprises a sequence for correction or modification of a defective or deleterious gene.

5. A method of increasing cell proliferation in a mammalian cell, comprising:
obtaining a cell;
contacting said cell with a nucleic acid sequence encoding a protein selected from the group consisting of EVI-1, PRDM16, SETBP1, and an active fragment thereof;
allowing said nucleic acid to enter the cell; and
allowing said cell to proliferate;
wherein said cell containing said nucleic acid proliferates at an increased rate compared to a cell that has not been contacted with said nucleic acid sequence.

6. The method of claim 5, wherein said proliferation occurs in a cell culture.

7. The method of claim 5, wherein said proliferation occurs in vivo.

8. The method of claim 5, wherein said nucleic acid integrates into chromosomal DNA.

9. The method of claim 5, wherein said nucleic acid is present in the cytoplasm of the cell.

10. The method of claim 5, wherein said nucleic acid is operably linked to a promoter.

11. The method of claim 5, wherein said nucleic acid is constitutively expressed.

12. The method of claim 5, wherein expression of said nucleic acid is inducible by an exogenously added agent.

13. The method of claim 5, wherein said nucleic acid is conditionally expressed.

14. The method of claim 5, wherein said nucleic acid is present in a vector.

15. The method of claim 14, wherein said vector is a viral vector.

16. The method of claim 5, wherein said nucleic acid is expressed for a number of division cycles selected from the group consisting of: about 1, 3, 5, 8, 10, 13, 17, or 20 division cycles, then expression decreases or stops thereafter.

17. The method of claim 5, wherein said cell is a cell selected from the group consisting of a hematopoietic stem cell, hematopoietic progenitor cell, a stem cell, an embryonic stem cell, an adult stem cell, a multipotent stem cell, and a myelopoietic stem cell.

18. The method of claim 17, wherein said cell is a hematopoietic stem cell.

19. A method of expansion of a gene-corrected cell, comprising:
obtaining a cell in need of gene correction;
contacting said cell with a functional copy of a said gene in need of correction;
contacting said cell with a copy of a nucleic acid encoding a polypeptide sequence selected from the group consisting of EVI-1, PRDM16, SETBP1, and an active fragment thereof; and
allowing said cell to proliferate in culture;
thereby obtaining an expanded culture of gene corrected cells.

20. A method of forming a bodily tissue having gene corrected cells, comprising:
obtaining a cell in need of gene correction;
contacting said cell with a functional copy of a said gene in need of correction;
contacting said cell with a copy of a nucleic acid encoding a polypeptide sequence selected from the group consisting of EVI-1, PRDM16, SETBP1, and a fragment thereof;
allowing said cell to proliferate in culture; and
treating said cell culture to allow formation of a bodily tissue;
thereby obtaining an expanded culture of gene corrected cells.

21. A method of identifying a gene, the modulation of which increases the proliferation rate of a cell, comprising:
obtaining a sample of cells from a patient having previously received a therapeutic transfection with a nucleic acid sequence;
identifying positions of nucleic acid insertion in the cells from the sample;
identifying a favorable insertion site based upon disproportional representation of said site in the population of transfected cells; and
identifying a gene associated with the insertion site.

22. A nucleic acid integration region that, when insertionally modulated, results in increased hematopoietic cell proliferation, comprising a sequence selected from the group consisting of: the EVI-1 gene, the PRDM16 gene, and the SETBP1 gene.

23. A method of identifying a favorable insertion site of a nucleic acid sequence in a proliferating cell culture, comprising:
transfecting a cell sample with a nucleic acid sequence;
allowing cell proliferation to occur;
determining at least one main insertion site of the nucleic acid using LAM-PCR over time;
using said at least one main insertion site to predict the location of at least one main insertion site of another cell sample transfected with a substantially similar nucleic acid sequence over a similar time period;
obtaining a sample of cells from a patient having previously received a therapeutic transfection with a nucleic acid sequence;
identifying positions of nucleic acid insertion in the cells from the sample; and
identifying a favorable insertion site based upon disproportional representation of said site in the population of transfected cells.

24. A method of expansion of a cell, comprising contacting said cell with a polypeptide selected from the group consisting of: an EVI-1 polypeptide, a PRDM16 polypeptide, a SETBP1 polypeptide, an active fragment thereof, or a synthetic peptide derivative thereof.


SEQUENCE LISTING (161 sequences)

SEQ ID NO: 1 - DNA, 22 bp, Artificial Sequence
  Oligonucleotide primers
ggttttggcgatctcaacagaa

SEQ ID NO: 2 - DNA, 25 bp, Artificial Sequence
  Oligonucleotide primers
tgtattgtcccacttccattttgaa

SEQ ID NO: 3 - DNA, 29 bp, Artificial Sequence
  Oligonucleotide primers
tcatcaccaaggtggtcactcaccctttc

SEQ ID NO: 4 - DNA, 24 bp, Artificial Sequence
  Oligonucleotide primers
ctgctgccagctttgagtacacta

SEQ ID NO: 5 - DNA, 25 bp, Artificial Sequence
  Oligonucleotide primers
gagatgccagagtcagataccacaa

SEQ ID NO: 6 - DNA, 23 bp, Artificial Sequence
  Oligonucleotide primers
accccagctcccagctcttgcgt

SEQ ID NO: 7 - DNA, 24 bp, Artificial Sequence
  Oligonucleotide primers
ttgtacgtgggcagaccgcagaga

SEQ ID NO: 8 - DNA, 24 bp, Artificial Sequence
  Oligonucleotide primers
ccaaagggcccatcaaccgctatc

SEQ ID NO: 9 - DNA, 23 bp, Artificial Sequence
  Oligonucleotide primers
ggatagtgggtcccatgtttctg

SEQ ID NO: 10 - DNA, 24 bp, Artificial Sequence
  Oligonucleotide primers
ccgctatcttaggtagtttccacg

SEQ ID NO: 11 - DNA, 22 bp, Artificial Sequence
  Oligonucleotide primers
gagccggggacagatgatgagg

SEQ ID NO: 12 - DNA, 21 bp, Artificial Sequence
  Oligonucleotide primers
gcggctgggataaggctgttc

SEQ ID NO: 13 - DNA, 22 bp, Artificial Sequence
  Oligonucleotide primers
gtttggcccaacgttagctatt

SEQ ID NO: 14 - DNA, 20 bp, Artificial Sequence
  Oligonucleotide primers
gcccttgatctgaacttctc

SEQ ID NO: 15 - DNA, 21 bp, Artificial Sequence
  Oligonucleotide primers
ttccatgccttgcaaaatggc

SEQ ID NO: 16 - DNA, 20 bp, Artificial Sequence
  Oligonucleotide primers
gacccgggagatctgaattc

SEQ ID NO: 17 - DNA, 21 bp, Artificial Sequence
  Oligonucleotide primers
gatctgaattcagtggcacag

SEQ ID NO: 18 - DNA, 20 bp, Artificial Sequence
  Oligonucleotide primers
tcgccgctggcctgctaaat

SEQ ID NO: 19 - DNA, 20 bp, Artificial Sequence
  Oligonucleotide primers
ctgctaaatgaatctgaggg

SEQ ID NO: 20 - DNA, 20 bp, Artificial Sequence
  Oligonucleotide primers
ctgctaaatgaatctgaggg

SEQ ID NO: 21 - DNA, 20 bp, Artificial Sequence
  Oligonucleotide primers
aatgaatctgagggcagctg

SEQ ID NO: 22 - DNA, 20 bp, Artificial Sequence
  Oligonucleotide primers
ttgcacctggagctcggctc

SEQ ID NO: 23 - DNA, 20 bp, Artificial Sequence
  Oligonucleotide primers
aagcagggcgacaagaggtt

SEQ ID NO: 24 - DNA, 20 bp, Artificial Sequence
  Oligonucleotide primers
gtcgtcgtgttggtaatccc

SEQ ID NO: 25 - DNA, 20 bp, Artificial Sequence
  Oligonucleotide primers
tgagggcactgctcgtgtgg

SEQ ID NO: 26 - DNA, 20 bp, Artificial Sequence
  Oligonucleotide primers
taaggagcgcgtcgaggggg

SEQ ID NO: 27 - DNA, 20 bp, Artificial Sequence
  Oligonucleotide primers
ggcttcggcctccaacccga

SEQ ID NO: 28 - DNA, 20 bp, Artificial Sequence
  Oligonucleotide primers
ttgcgagctccgtgcagtta

SEQ ID NO: 29 - DNA, 20 bp, Artificial Sequence
  Oligonucleotide primers
acaagatgccatgttaatta

SEQ ID NO: 30 - DNA, 20 bp, Artificial Sequence
  Oligonucleotide primers
tgcgagctccgtgcagttac

SEQ ID NO: 31 - DNA, 20 bp, Artificial Sequence
  Oligonucleotide primers
tccaaataacaagatgccat

SEQ ID NO: 32 - DNA, 20 bp, Artificial Sequence
  Oligonucleotide primers
taaataagtgttttccttac

SEQ ID NO: 33 - DNA, 20 bp, Artificial Sequence
  Oligonucleotide primers
taagtgttttccttacgact

SEQ ID NO: 34 - DNA, 20 bp, Artificial Sequence
  Oligonucleotide primers
agaggcttctgtttccgcag

SEQ ID NO: 35 - DNA, 20 bp, Artificial Sequence
  Oligonucleotide primers
tgctccccacctaacactcg

SEQ ID NO: 36 - DNA, 20 bp, Artificial Sequence
  Oligonucleotide primers
tttatgttatcgaggcagaa

SEQ ID NO: 37 - DNA, 20 bp, Artificial Sequence
  Oligonucleotide primers
atgttatcgaggcagaattc

SEQ ID NO: 38 - DNA, 20 bp, Artificial Sequence
  Oligonucleotide primers
tatgttatcgaggcagaatt

SEQ ID NO: 39 - DNA, 20 bp, Artificial Sequence
  Oligonucleotide primers
cgattcagtggcagtgagcc

SEQ ID NO: 40 - DNA, 20 bp, Artificial Sequence
  Oligonucleotide primers
tagactgtgaccctgaagac

SEQ ID NO: 41 - DNA, 20 bp, Artificial Sequence
  Oligonucleotide primers
actaagggtgatttgctttg

SEQ ID NO: 42 - DNA, 20 bp, Artificial Sequence
  Oligonucleotide primers
gattagctatgtatactgca

SEQ ID NO: 43 - DNA, 20 bp, Artificial Sequence
  Oligonucleotide primers
gtaatttgttaccctcttta

SEQ ID NO: 44 - DNA, 20 bp, Artificial Sequence
  Oligonucleotide primers
gttctcagaaacccaagaca

SEQ ID NO: 45 - DNA, 20 bp, Artificial Sequence
  Oligonucleotide primers
cagtgcctaagctgactttg

SEQ ID NO: 46 - DNA, 20 bp, Artificial Sequence
  Oligonucleotide primers
gtagatgtttggtttacttc

SEQ ID NO: 47 - DNA, 20 bp, Artificial Sequence
  Oligonucleotide primers
cacataggtgcttctgtatg

SEQ ID NO: 48 - DNA, 20 bp, Artificial Sequence
  Oligonucleotide primers
ctttcatgagaaacaaggcc

SEQ ID NO: 49 - DNA, 20 bp, Artificial Sequence
  Oligonucleotide primers
ggatttcagaaccctatctt

SEQ ID NO: 50 - DNA, 20 bp, Artificial Sequence
  Oligonucleotide primers
agaactgagtattattactg

SEQ ID NO: 51 - DNA, 20 bp, Artificial Sequence
  Oligonucleotide primers
atcaagaacatcttgtgaat

SEQ ID NO: 52 - DNA, 20 bp, Artificial Sequence
  Oligonucleotide primers
ctgccttcattgtgtaactg

SEQ ID NO: 53 - DNA, 20 bp, Artificial Sequence
  Oligonucleotide primers
gtaagaagttagtgctccag

SEQ ID NO: 54 - DNA, 20 bp, Artificial Sequence
  Oligonucleotide primers
gatggagtagaaactgtctg

SEQ ID NO: 55 - DNA, 20 bp, Artificial Sequence
  Oligonucleotide primers
gtttgagccatgcaaatctg

SEQ ID NO: 56 - DNA, 20 bp, Artificial Sequence
  Oligonucleotide primers
taacataaataagtctttag

SEQ ID NO: 57 - DNA, 20 bp, Artificial Sequence
  Oligonucleotide primers
cataaataagtctttaggtt

SEQ ID NO: 58 - DNA, 20 bp, Artificial Sequence
  Oligonucleotide primers
ggagacacatcaaggaactt

SEQ ID NO: 59 - DNA, 20 bp, Artificial Sequence
  Oligonucleotide primers
atgtattgcaactggcatag

SEQ ID NO: 60 - DNA, 20 bp, Artificial Sequence
  Oligonucleotide primers
taaggttacatcccacagct

SEQ ID NO: 61 - DNA, 20 bp, Artificial Sequence
  Oligonucleotide primers
ccagatgaagttagtttttg

SEQ ID NO: 62 - DNA, 20 bp, Artificial Sequence
  Oligonucleotide primers
ccagatgaagttagtttttg

SEQ ID NO: 63 - DNA, 20 bp, Artificial Sequence
  Oligonucleotide primers
agaaaatgggtgtatgatga

SEQ ID NO: 64 - DNA, 20 bp, Artificial Sequence
  Oligonucleotide primers
aattatacaacattggtgta

SEQ ID NO: 65 - DNA, 20 bp, Artificial Sequence
  Oligonucleotide primers
atgtcaccaatgtaatgaca

SEQ ID NO: 66 - DNA, 20 bp, Artificial Sequence
  Oligonucleotide primers
agtattgcatatctatatga

SEQ ID NO: 67 - DNA, 20 bp, Artificial Sequence
  Oligonucleotide primers
tctacacagtaatgtattta

SEQ ID NO: 68 - DNA, 20 bp, Artificial Sequence
  Oligonucleotide primers
cttcctcacagaaggattgg

SEQ ID NO: 69 - DNA, 20 bp, Artificial Sequence
  Oligonucleotide primers
tattgacaccactttctagc

SEQ ID NO: 70 - DNA, 20 bp, Artificial Sequence
  Oligonucleotide primers
tattgacaccactttctagc

SEQ ID NO: 71 - DNA, 20 bp, Artificial Sequence
  Oligonucleotide primers
taggacgatatcaatactta

SEQ ID NO: 72 - DNA, 20 bp, Artificial Sequence
  Oligonucleotide primers
tagatgaagaaaattcactc

SEQ ID NO: 73 - DNA, 20 bp, Artificial Sequence
  Oligonucleotide primers
ttgccaagtgttgaggtgca

SEQ ID NO: 74 - DNA, 20 bp, Artificial Sequence
  Oligonucleotide primers
ttgccaagtgttgaggtgca

SEQ ID NO: 75 - DNA, 20 bp, Artificial Sequence
  Oligonucleotide primers
tgagcgaaaattgtagaaca

SEQ ID NO: 76 - DNA, 20 bp, Artificial Sequence
  Oligonucleotide primers
tgaacaagagtagtgtcaca

SEQ ID NO: 77 - DNA, 20 bp, Artificial Sequence
  Oligonucleotide primers
gatgtcaacagagcattgag

SEQ ID NO: 78 - DNA, 20 bp, Artificial Sequence
  Oligonucleotide primers
cgtcttgtaactctctcaag

SEQ ID NO: 79 - DNA, 20 bp, Artificial Sequence
  Oligonucleotide primers
gcttgatgtttagtctgtgc

SEQ ID NO: 80 - DNA, 20 bp, Artificial Sequence
  Oligonucleotide primers
acaggcaataaagttcagga

SEQ ID NO: 81 - DNA, 20 bp, Artificial Sequence
  Oligonucleotide primers
agcccaggactcatttctcg

SEQ ID NO: 82 - DNA, 20 bp, Artificial Sequence
  Oligonucleotide primers
agcccaggactcatttctcg

SEQ ID NO: 83 - DNA, 20 bp, Artificial Sequence
  Oligonucleotide primers
gtgtgccttgatcgctcaag

SEQ ID NO: 84 - DNA, 20 bp, Artificial Sequence
  Oligonucleotide primers
gagcagttacagaggcttgt

SEQ ID NO: 85 - DNA, 20 bp, Artificial Sequence
  Oligonucleotide primers
ctgcaccagtaacacagtga

SEQ ID NO: 86 - DNA, 20 bp, Artificial Sequence
  Oligonucleotide primers
ataccaacaggtacgactgg

SEQ ID NO: 87 - DNA, 20 bp, Artificial Sequence
  Oligonucleotide primers
gtattctcaatgattcccct

SEQ ID NO: 88 - DNA, 20 bp, Artificial Sequence
  Oligonucleotide primers
tgcttttcttcaaaggatgg

SEQ ID NO: 89 - DNA, 20 bp, Artificial Sequence
  Oligonucleotide primers
aaggatgggttggagcgtta

SEQ ID NO: 90 - DNA, 20 bp, Artificial Sequence
  Oligonucleotide primers
ccgaactgcacagctcagca

SEQ ID NO: 91 - DNA, 20 bp, Artificial Sequence
  Oligonucleotide primers
ctcagcaaaagcgccctcgc

SEQ ID NO: 92 - DNA, 20 bp, Artificial Sequence
  Oligonucleotide primers
ctcagcaaaagcgccctcgc

SEQ ID NO: 93 - DNA, 21 bp, Artificial Sequence
  Oligonucleotide primers
tcgccctccgcgcgccgcctc

SEQ ID NO: 94 - DNA, 19 bp, Artificial Sequence
  Oligonucleotide primers
taacgctccaacccatcct

SEQ ID NO: 95 - DNA, 19 bp, Artificial Sequence
  Oligonucleotide primers
agcattgatcggagagacg

SEQ ID NO: 96 - DNA, 20 bp, Artificial Sequence
  Oligonucleotide primers
aggcagtagtgtcggttaag

SEQ ID NO: 97 - DNA, 20 bp, Artificial Sequence
  Oligonucleotide primers
gctaggcaagtgaagggctg

SEQ ID NO: 98 - DNA, 20 bp, Artificial Sequence
  Oligonucleotide primers
cttcaaccagctccgccatg

SEQ ID NO: 99 - DNA, 20 bp, Artificial Sequence
  Oligonucleotide primers
accagtgcctattcaagcct

SEQ ID NO: 100 - DNA, 20 bp, Artificial Sequence
  Oligonucleotide primers
ggtcctttctaattgacgcg

SEQ ID NO: 101 - DNA, 20 bp, Artificial Sequence
  Oligonucleotide primers
ttcagagacgcagccacaga

SEQ ID NO: 102 - DNA, 20 bp, Artificial Sequence
  Oligonucleotide primers
tggtctccttagaggcttct

SEQ ID NO: 103 - DNA, 20 bp, Artificial Sequence
  Oligonucleotide primers
gaggcagccacagaaggagg

SEQ ID NO: 104 - DNA, 20 bp, Artificial Sequence
  Oligonucleotide primers
ctgcgtctctgaaaggatcc

SEQ ID NO: 105 - DNA, 20 bp, Artificial Sequence
  Oligonucleotide primers
agaaaggacccgttggccac

SEQ ID NO: 106 - DNA, 20 bp, Artificial Sequence
  Oligonucleotide primers
aggagttaaggagcgcgtcg

SEQ ID NO: 107 - DNA, 20 bp, Artificial Sequence
  Oligonucleotide primers
ccaacccgactttgtttgcg

SEQ ID NO: 108 - DNA, 20 bp, Artificial Sequence
  Oligonucleotide primers
ttgcacctggagctcggctc

SEQ ID NO: 109 - DNA, 20 bp, Artificial Sequence
  Oligonucleotide primers
caagaggttctggctggtgg

SEQ ID NO: 110 - DNA, 20 bp, Artificial Sequence
  Oligonucleotide primers
aatgcacaggcctgccttta

SEQ ID NO: 111 - DNA, 20 bp, Artificial Sequence
  Oligonucleotide primers
cgctgattttcctccagcgg

SEQ ID NO: 112 - DNA, 20 bp, Artificial Sequence
  Oligonucleotide primers
gaagctatttccttagacag

SEQ ID NO: 113 - DNA, 20 bp, Artificial Sequence
  Oligonucleotide primers
taagaacgggacttgtagcc

SEQ ID NO: 114 - DNA, 20 bp, Artificial Sequence
  Oligonucleotide primers
ctgcctttccactgatagtt

SEQ ID NO: 115 - DNA, 20 bp, Artificial Sequence
  Oligonucleotide primers
gaaggaacacactcctggcc

SEQ ID NO: 116 - DNA, 20 bp, Artificial Sequence
  Oligonucleotide primers
tgaaagggtatgcttgaaag

SEQ ID NO: 117 - DNA, 20 bp, Artificial Sequence
  Oligonucleotide primers
acgtctctctgcaaatatga

SEQ ID NO: 118 - DNA, 20 bp, Artificial Sequence
  Oligonucleotide primers
acgtaagacaactccacagt

SEQ ID NO: 119 - DNA, 20 bp, Artificial Sequence
  Oligonucleotide primers
ccacatcagagtcaagaaga

SEQ ID NO: 120 - DNA, 20 bp, Artificial Sequence
  Oligonucleotide primers
ccacatcagagtcaagaaga

SEQ ID NO: 121 - DNA, 20 bp, Artificial Sequence
  Oligonucleotide primers
ctaattactgagatagctcc

SEQ ID NO: 122 - DNA, 20 bp, Artificial Sequence
  Oligonucleotide primers
ccattatgttcctcattgca

SEQ ID NO: 123 - DNA, 20 bp, Artificial Sequence
  Oligonucleotide primers
gagcaaacttcaaaggaagc

SEQ ID NO: 124 - DNA, 20 bp, Artificial Sequence
  Oligonucleotide primers
aagaagagggtgggcccaag

SEQ ID NO: 125 - DNA, 20 bp, Artificial Sequence
  Oligonucleotide primers
gtactttgtgcccaacttgc

SEQ ID NO: 126 - DNA, 20 bp, Artificial Sequence
  Oligonucleotide primers
gaatgctgcaactgcaagga

SEQ ID NO: 127 - DNA, 20 bp, Artificial Sequence
  Oligonucleotide primers
cagtcagcatggaaatgatt

SEQ ID NO: 128 - DNA, 20 bp, Artificial Sequence
  Oligonucleotide primers
cagtcagcatggaaatgatt

SEQ ID NO: 129 - DNA, 20 bp, Artificial Sequence
  Oligonucleotide primers
gtcctctcttcattgtgtca

SEQ ID NO: 130 - DNA, 20 bp, Artificial Sequence
  Oligonucleotide primers
gctctccttcagcatgtcaa

SEQ ID NO: 131 - DNA, 20 bp, Artificial Sequence
  Oligonucleotide primers
gagattcacacagtaaaaga

SEQ ID NO: 132 - DNA, 20 bp, Artificial Sequence
  Oligonucleotide primers
caggctaacttctcgactct

SEQ ID NO: 133 - DNA, 20 bp, Artificial Sequence
  Oligonucleotide primers
caactggcctgaattagagt

SEQ ID NO: 134 - DNA, 20 bp, Artificial Sequence
  Oligonucleotide primers
caggacccttcacggatacc

SEQ ID NO: 135 - DNA, 20 bp, Artificial Sequence
  Oligonucleotide primers
ggcatagcatttgcatataa

SEQ ID NO: 136 - DNA, 21 bp, Artificial Sequence
  Oligonucleotide primers
agaggagaacggccagtattg

SEQ ID NO: 137 - DNA, 22 bp, Artificial Sequence
  Oligonucleotide primers
actccactacctcgcaggcatt

SEQ ID NO: 138 - DNA, 21 bp, Artificial Sequence
  Oligonucleotide primers
tgtccgtttcctcctgcgcgg

SEQ ID NO: 139 - DNA, 27 bp, Artificial Sequence
  Oligonucleotide primers
aactggaagaattcgcggccgcaggaa

SEQ ID NO: 140 - DNA, 21 bp, Artificial Sequence
  Oligonucleotide primers
tcctgtggcatccacgaaact

SEQ ID NO: 141 - DNA, 21 bp, Artificial Sequence
  Oligonucleotide primers
gaagcatttgcggtggacgat

SEQ ID NO: 142 - DNA, 20 bp, Artificial Sequence
  Oligonucleotide primers
tggagaaacacatgctgtca

SEQ ID NO: 143 - DNA, 20 bp, Artificial Sequence
  Oligonucleotide primers
ataaagggcttcacactgct

SEQ ID NO: 144 - DNA, 20 bp, Artificial Sequence
  Oligonucleotide primers
gccacatccagtgaagcatt

SEQ ID NO: 145 - DNA, 20 bp, Artificial Sequence
  Oligonucleotide primers
tgagccagcttccaacatct

SEQ ID NO: 146 - DNA, 20 bp, Artificial Sequence
  Oligonucleotide primers
aggagggttctccttacaaa

SEQ ID NO: 147 - DNA, 20 bp, Artificial Sequence
  Oligonucleotide primers
tgactggcatctatgcagaa

SEQ ID NO: 148 - DNA, 21 bp, Artificial Sequence
  Oligonucleotide primers
ctgacggacgtggaagtgtcg

SEQ ID NO: 149 - DNA, 21 bp, Artificial Sequence
  Oligonucleotide primers
cagggggtagacgccttcctt

SEQ ID NO: 150 - DNA, 20 bp, Artificial Sequence
  Oligonucleotide primers
tctccgaagacctgggcagt

SEQ ID NO: 151 - DNA, 20 bp, Artificial Sequence
  Oligonucleotide primers
cacctggctcaatgtcctta

SEQ ID NO: 152 - DNA, 22 bp, Artificial Sequence
  Oligonucleotide primers
ccccagatcagccaactcacca

SEQ ID NO: 153 - DNA, 20 bp, Artificial Sequence
  Oligonucleotide primers
ggtgccggtccaggttggtc

SEQ ID NO: 154 - DNA, 20 bp, Artificial Sequence
  Oligonucleotide primers
acacctgaggacgcacactg

SEQ ID NO: 155 - DNA, 20 bp, Artificial Sequence
  Oligonucleotide primers
ggttgcacaggtggcacttg

SEQ ID NO: 156 - DNA, 20 bp, Artificial Sequence
  Oligonucleotide primers
taaaagtggaccagacagca

SEQ ID NO: 157 - DNA, 20 bp, Artificial Sequence
  Oligonucleotide primers
tcacgaagttgttgcctgtt

SEQ ID NO: 158 - DNA, 20 bp, Artificial Sequence
  Oligonucleotide primers
taaaagtggaccagacagca

SEQ ID NO: 159 - DNA, 20 bp, Artificial Sequence
  Oligonucleotide primers
tcacgaagttgttgcctgtt

SEQ ID NO: 160 - DNA, 18 bp, Artificial Sequence
  Oligonucleotide primers
tccgatagactgcgtcgc

SEQ ID NO: 161 - DNA, 20 bp, Artificial Sequence
  Oligonucleotide primers
tcttgctgtttgcatccgaa